(19) United States
(12) Patent Application Publication
(10) Pub. No.: US 2022/0301656 A1
(43) Pub. Date: Sep. 22, 2022

(54) GENOME SEQUENCING AS AN ALTERNATIVE TO CYTOGENETIC ANALYSIS

(71) Applicant: Washington University, St. Louis, MO (US)

(72) Inventors: Eric Duncavage, St. Louis, MO (US); David Spencer, St. Louis, MO (US)

(73) Assignee: Washington University, St. Louis, MO (US)

(21) Appl. No.: 17/699,053

(22) Filed: Mar. 18, 2022

Related U.S. Application Data
(60) Provisional application No. 63/162,665, filed on Mar. 18, 2021.

(51) Int. Cl. G16B 20/20; G16B 20/10

(52) U.S. Cl. CPC G16B 20/20; G16B 20/10

(57) ABSTRACT

A computer-implemented method for the identification of clinically relevant structural variants in a subject with AML or MDS from whole genome sequencing data is disclosed that includes providing a whole-genome sequencing dataset, performing a structural variant analysis on the whole-genome sequencing dataset and producing a report that includes clinically relevant CNAs, SVs, and gene-level variants identified by the structural variant analysis.

### CROSS-REFERENCE TO RELATED APPLICATIONS

This application claims priority to U.S. provisional application No. 63/162,665 filed on Mar. 18, 2021, the content of which is incorporated by reference in its entirety.

### STATEMENT REGARDING FEDERALLY SPONSORED RESEARCH OR DEVELOPMENT

Not applicable.

### MATERIAL INCORPORATED-BY-REFERENCE

Not applicable.

### FIELD OF THE DISCLOSURE

The present disclosure generally relates to methods of genomic profiling using whole-genome sequencing.

### BACKGROUND OF THE DISCLOSURE

Chromosome analysis has been used for cancer diagnosis and to guide treatment decisions for over 40 years. The discovery of chromosome-level mutations like the BCR-ABL1 fusion gene in chronic myeloid leukemia (CML) and PML-RARA gene fusion in AML have transformed these once lethal cancers into diseases that can be essentially cured with targeted therapies. Over a thousand chromosome-level mutations have now been identified across numerous cancer types, and although more recent genomic studies of cancer have revealed many additional nucleotide-level cancer-associated mutations, cytogenetic mutations still account for the majority of clinically-relevant genomic changes in cancer. For example, FISH and karyotyping are required for the risk classification system in AML, and cytogenetic testing for chromosomal rearrangements facilitates accurate diagnosis of B-cell lymphomas and guides therapy in non-small cell lung cancer. Moreover, the oncogenes targeted by many of the FDA-approved cancer therapies result from chromosomal rearrangements that are routinely detected using karyotyping or FISH.

While effective, conventional cytogenetic methods are imprecise. Karyotyping depends on identifying changes in chromosomes using their unique banding patterns, meaning that small rearrangements can be missed and complex structural mutations may obscure important findings that are clinically actionable. Perhaps the most limiting aspect of conventional cytogenetic testing is that it requires culturing of live cells under stimulating conditions, which makes the method essentially unavailable to the 90% of cancer patients with solid tumors. Certain chromosomal rearrangements can be tested for using FISH, which is routinely performed on formalin-fixed biopsies of solid tumors. However, this approach uses locus-specific DNA probes that can miss rearrangements with atypical breakpoints or identify changes that do not result in the expected mutant gene products and may therefore not result in the anticipated clinical outcome. The most limiting aspect of FISH testing is that it is impractical to test clinical samples for more than a few specific mutations at a time, which can result in testing that is incomplete if sample amounts are insufficient. The need for multiplex testing has led to the development of targeted sequencing panels (e.g., the Foundation of Medicine Comprehensive Cancer Panel, some of which can detect both copy number mutations (e.g., gene deletions and amplifications) and selected gene fusion events. Although these technologies can accurately identify an expanding number of mutations, they require complicated laboratory procedures, often involving both DNA and RNA, and take multiple days to complete. In addition, the assays must predefine the genomic regions that are selected for targeted sequencing and can therefore only identify chromosomal rearrangements that occur at these specific loci. As a result, relatively few tumors are ever tested for the full range of clinically relevant chromosome-level mutations, including those that may respond to approved targeted therapies. To expand precision medicine, new methods are needed that can be applied to more cancer samples and that test for more mutations, including those that are known to predict response to targeted therapies.

Genetic profiling is a routine component of the diagnostic workup for an increasing number of cancers and is used to predict clinical outcomes and responses to targeted therapies. Mutations that are clinically actionable for any individual type of cancer typically span a wide range of genomic events, including chromosomal rearrangements, gene amplifications and deletions, and single-nucleotide changes. The diversity of these findings necessitates the use of multiple platforms to obtain the genomic information needed for clinical management. Whole-genome sequencing is an unbiased method of detecting all types of mutations that could potentially be used to replace current testing algorithms. Such sequencing can also be performed on a limited amount of DNA to identify genomic changes that may be cryptic in other types of analyses. These features of whole-genome sequencing suggest that it could improve genomic profiling in patients with cancer.

Genomic abnormalities are particularly important for diagnostic classification and risk assessment in patients with acute myeloid leukemia (AML) and myelodysplastic syndromes (MDS). Recurrent chromosomal abnormalities are the basis for the AML genomic classification system of the World Health Organization, and the association of these alterations and certain genetic mutations with clinical outcomes has led to the development of algorithms for genetic risk stratification in patients with AML. Similar studies involving patients with MDS have resulted in the cytogenetic component of the International Prognostic Scoring System-Revised (IPSS-R) in such patients. Although advances in sequencing technology have improved the ability to identify genetic mutations, the detection of chromosomal rearrangements is primarily performed through conventional metaphase cytogenetic analysis (i.e., karyotyping). The latter approach is effective but has several limitations, including the need to obtain viable cells, low sensitivity, and limited resolution.

Fluorescence in situ hybridization (FISH) and targeted sequencing assays that use DNA, RNA, or both are also used, but these methods are informative only in the regions selected for analysis and may provide incomplete information regarding identified chromosomal rearrangements. As a result, conventional cytogenetic analysis remains an essential component of the diagnostic workup for patients with AML or MDS.

The importance of genetic profiling in such patients and the variety of clinically relevant mutation types suggest that whole-genome sequencing could be used in place of standard testing approaches. Although the high cost of sequencing and complex, time-consuming analysis methods have historically restricted such sequencing to research studies, recent advances have made this analysis simpler to perform, faster, and less expensive.

Other objects and features will be in part apparent and in part pointed out hereinafter.

### SUMMARY OF THE DISCLOSURE

In one aspect, a computer-implemented method for the identification of clinically relevant structural variants in a subject with AML or MDS from whole genome sequencing data is disclosed that includes providing a whole-genome sequencing dataset, the whole-genome sequencing dataset comprising a plurality of alignments of tumor DNA sequence fragments to a reference human genome to a computing device; performing, using the computing device, a structural variant analysis on the whole-genome sequencing dataset, the structural variant analysis including copy-number alteration (CNA) identification, structural variant (SV) identification, and gene-level variant identification to identify clinically relevant structural variants indicative of AML or MDS within the whole-genome sequencing dataset; and producing, using the computing device, a report comprising the clinically relevant CNAs, SVs, and gene-level variants identified by the structural variant analysis. In some aspects, copy-number alteration (CNA) identification further comprises transforming, using the computing device, the alignments of the whole-genome sequencing dataset into a plurality of read counts over 500,000 bp nonoverlapping windows across the genome; transforming, using the computing device, the plurality of read counts into a plurality of CNAs; and filtering, using the computing device, plurality of CNAs to retain only CNAs greater than 5 Mbp. In some aspects, SV identification further comprises: transforming, using the computing device, the alignments of the whole-genome sequencing dataset into a plurality of SV calls; filtering, using the computing device, the plurality of SVs to retain only SV calls greater than 100 kbp in length; and filtering, using the computing device, the SV calls greater than 100 kbp in length to identify translocations, deletions, duplications, and inversions that overlap a predefined list of recurrent and/or risk-defining SVs associated with AML or MDS. In some aspects, gene-level variant identification further comprises identifying, using the computing device, the alignments of the whole-genome sequencing dataset within about 85 kbp targeting 40 predetermined genes and gene hotspots that are recurrently mutated in AML or MDS. In some aspects, the clinically relevant CNAs, SVs, and gene-level variants identified by the structural variant analysis are indicative of a clinical outcome of the subject. In some aspects, providing the whole-genome sequencing dataset whole genome sequencing data further comprising performing whole-genome sequencing on a biological sample comprising tumor DNA from the subject with about 60× genome coverage.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 is a block diagram schematically illustrating a system in accordance with one aspect of the disclosure.

FIG. 2 is a block diagram schematically illustrating a computing device in accordance with one aspect of the disclosure.

FIG. 3 is a block diagram schematically illustrating a remote or user computing device in accordance with one aspect of the disclosure.

FIG. 4 is a block diagram schematically illustrating a server system in accordance with one aspect of the disclosure.

FIG. 5 is a schematic illustration of the workflow and approximate processing time for each step of the rapid WGS method and analysis of samples obtained from the study patients. (An example of the reports that were generated by this process are provided in FIG. 9).

FIG. 6A is a comparison of WGS with Conventional Cytogenetic Analysis and Targeted Gene Sequencing. It shows the sensitivity of WGS for the detection of recurrent structural variants (SVs) and copy-number alterations (CNAs) as compared with conventional cytogenetic analysis and for the detection of single-nucleotide variants (SNVs) and insertion-deletions (INDELs) as compared with high-coverage targeted gene sequencing. Error bars denote 95% confidence intervals.

FIG. 6B shows the identification and confirmation by WGS of 13 new recurrent SVs that were not detected by conventional cytogenetic analysis, as supported by orthogonal methods, including fluorescence in situ hybridization (FISH), polymerase chain reaction (PCR) with sequencing of SV breakpoints, or detection of fusion transcripts in RNA-sequence (RNA-seq) data.

FIG. 6C shows the identification of 21 new CNAs in 14 patients; 12 of these alterations were confirmed by chromosomal microarray (CMA), FISH, or sequence-defined breakpoints. An additional 9 CNAs were identified by WGS only and could not be confirmed by CMA (in 6 patients) or confirmation was not attempted because of the size or abundance of the CNA event (in 3 patients). CNAs were also identified in 13 patients with ambiguous or inconclusive results on cytogenetic analysis. Additional details regarding these comparisons are provided in Tables S4 and S5 and FIGS. 18A, 18B, 18C, and 18D.

FIG. 7A describes is a bar graph summarizing the time it took to perform WGS-Based Genomic Profiling on samples obtained from 117 consecutive patients with AML or MDS by means of WGS, as indicated by the dashed horizontal black line. The height of each bar shows the total time in days for processing, starting from construction of the sequencing library and ending with completion of the automated final report for an individual patient sample. The duration of each individual step (as obtained from time stamps recorded in the information management system of the clinical laboratory) is indicated by the shaded bar segments and includes the duration of library generation and quality assessment, sequencing, and analysis and reporting. These times reflect the processing time plus waiting time before the next step. Longer turnaround times occurred because of delays between steps, rather than longer processing times. The dashed horizontal red lines show the recommended maximum turnaround time for FISH testing and conventional cytogenetic analysis, according to published recommendations, although shorter turnaround times occur in many laboratories.

FIG. 7B describes the Diagnostic Yield of WGS-based Genomic Profiling in 117 Consecutive Patients. It shows the yield of new WGS findings in samples obtained from 68 unselected, consecutive patients with AML. FIG. 7B shows the cumulative number of patients with new genomic findings that were identified by WGS, as compared with conventional cytogenetic analysis or FISH, performed at the time of diagnosis, along with the cumulative number of patients with new events that changed the category of genetic risk group on the basis of established European Leukemia Network (ELN) guidelines. FISH testing included assays for PML-RARA, CBFB-MYH11, RUNX1-RUNX1T1, del(5q), and chromosome 7 deletion, according to recommendations; all testing was performed in samples obtained from 60 of 68 patients (88%); subgroups of these assays were performed for the remaining patients. The results of ELN assignments to a genetic risk group by WGS, conventional cytogenetic analysis with FISH, and cytogenetic analysis alone are shown in the top panel. The red asterisk indicates that the patient's risk group was reclassified according to the WGS results, and the red arrow indicates that the risk-group assignment was based on FISH results alone.

FIG. 7B shows the genomic events that were detected by WGS and are labeled as concordant with cytogenetic analysis, with FISH, or with target sequencing (in black), new findings made by WGS (in blue), and new findings that resulted in a change in the ELN genetic risk group (in red). The status regarding internal tandem duplication in FLT3 (FLT3-ITD) and the allele ratio as determined by PCR were used for both conventional and WGS-based risk stratifications.

FIG. 8A is a risk assessment by WGS in Patients with AML, according to Existing Genetic Risk Groups. It shows overall survival for 71 patients with AML who were treated with chemotherapy alone after remission, as stratified into established ELN genetic risk groups on the basis of a combination of conventional cytogenetic analysis, FISH, and targeted gene sequencing.

FIG. 8B shows the same cohort as in FIG. 8A with risk stratification according to WGS results. The ratio of the mutated FLT3-ITD allele to the wild-type allele, as determined by PCR, was used for both the conventional and WGS classifications; the presence or absence of the mutation was used when allele ratios were not available.

FIG. 8C shows the clinical outcomes for 27 patients for whom genetic risk could not be determined because of inconclusive, unsuccessful, or unknown results on cytogenetic analysis. The median survival in this cohort was 11.2 months (95% confidence interval [CI], 5.6 to 38.8).

FIG. 8D shows the stratification of the cohort in FIG. 8C into established genetic risk groups with the use of WGS results, which predicted shorter overall survival for patients at adverse risk than for those at intermediate or favorable risk (not adverse) (age-adjusted hazard ratio for death for intermediate or favorable risk versus adverse risk, 0.29; 95% CI, 0.09 to 0.94). All P values were calculated with the use of a log-rank test for equal survival among the groups and adjusted for multiple comparisons.

FIG. 9 is an exemplary cover page of a graphical ChromoSeq WGS report highlighting the clinically significant findings in the genome sequence of an AML patient.

FIG. 10 is a histogram distribution of genome coverage of the genome-wide coverage depth in unique reads for 235 WGS cases.

FIG. 11 summarizes the variants detected per patient from a number distribution of SVs, CNAs, and gene mutations detected in 235 patients.

FIG. 12 is series of images confirming the SVs detected using the disclosed WGSA method. The images show FISH results from metaphase and interphase FISH analysis utilizing a dual color, break apart probe targeting KMT2A (manufactured by Vysis). A normal signal hybridization pattern is 2Y. Rearrangement of a KMT2A locus generates a separation of the green signal, which encompasses the 5′ segment of KMT2A and surrounding region, and the red signal, which encompasses the 3′ segment of KMT2A and surrounding region.

FIG. 13 is a series of images showing selected CNAs obtained by FISH. See also Table S5.

FIG. 14A is a first normalized coverage plot of WGS results for CNAs that were detected by WGS but could not be confirmed because of their small size, low abundance, lack of FISH probes, or lack of material.

FIG. 14B is a second normalized coverage plot of WGS results for CNAs that were detected by WGS but could not be confirmed because of their small size, low abundance, lack of FISH probes, or lack of material.

FIG. 14C is a third normalized coverage plot of WGS results for CNAs that were detected by WGS but could not be confirmed because of their small size, low abundance, lack of FISH probes, or lack of material.

FIG. 15A is a first normalized coverage plot of WGS results for CNAs that were detected by WGS.

FIG. 15B is a second normalized coverage plot of WGS results for CNAs that were detected by WGS.

FIG. 16 shows the sensitivity of WGS for mutations in AML risk-defining genes. Gold standard mutations NPM1 (NPM1c only), CEBPA, FLT3 (non-ITD mutations), RUNX1, and TP53 were obtained from targeted sequencing (>500× coverage) using a clinical assay (N=103 patients). FLT3-ITD mutations were obtained from clinical testing via PCR and capillary electrophoresis (N=104 patients). Numbers above each bar indicate the sensitivity (TP/(TP+FN)×100) and the number of true positives and total positives by the gold standard assay.

FIG. 17 compares FLT3-ITD detection by WGS vs. PCR. Results for 35 patients with FLT3-ITD mutations detected by either PCR and capillary electrophoresis or WGS. Rows show results for each patient with a positive ITD assay, including WGS, a clinical targeted sequencing assay, and PCR. The ITD allele sizes and allele ratios are indicated for each assay. Allele sizes and ratios were available for all positive results. Data from PCR include an in-house laboratory developed test (LDT) and the commercial FDA companion diagnostic assay, and therefore the allele size and ratios were not always reported. Note that ITD alleles were detected by WGS in two patients for whom the in-house LDT assay was negative (in 2002). ITD alleles were also detected in these patients in the AML TCGA study).

FIG. 18A shows the VAFs from deep targeted sequencing for 348 variants detected in 102 patients using a clinical targeted sequencing assay. Left panel shows the VAF from targeted sequencing for variants detected (N=300, in blue) and missed (N=48, in orange) by WGS.

FIG. 18B shows the distribution of VAFs for the detected and missed variants of FIG. 18A.

FIG. 18C shows the abundance and coverage depth of gene mutations in WGS data for 348 variants detected in 102 patients using a clinical targeted sequencing assay. Left panel shows WGS coverage for variants detected (N=300, in blue) and missed (N=48, in orange) by WGS. Right panels show the distribution of WGS coverage for detected and missed variants.

FIG. 18D shows the distribution of VAFs for the detected and missed variants of FIG. 18C.

FIG. 19A is a coverage metric for false negative gene mutations. It shows WGS coverage (indicated by the height of the bar) and variant supporting reads (height of the blue bar) for 48 variants that were detected by clinical targeted sequencing but missed by WGS. Most of the variants (41/48) were present in the WGS data, but at very low frequency. Detection of gene variants required >3 variant-supporting reads and >6× total coverage. Variants highlighted by the asterisk were subsequently detected upon top-up sequencing of 4 cases.

FIG. 19B is a coverage metric for false negative gene mutations. It demonstrates the theoretical binomial sampling probability for detecting variants at VAFs ranging from 2% to 20% and coverage levels from 0 to 100×. Note that at 50× coverage (the mean obtained for this study), there is a 45% probability of sampling a variant >3 times if the true abundance is 5%. This is consistent with previous work showing that ‘standard coverage’ WGS is inadequate for robust detection of low frequency variants, but this can be improved by increasing coverage depth (see also FIG. 20).

FIG. 20 shows additional variants detected after top-up sequencing. WGS missed 11 gene variants from patients 312088, 681540, 416413, and 262878 that were either present at low abundance in targeted sequencing data (<20% VAF, N=7) or occurred at position with low coverage in the WGS data (<25×, N=4). Additional sequencing of these samples increased the coverage for these samples from a mean of 35× (range: 17×-58×) to 83× (range: 59×-121×), and resulted in the detection of 9 of the 11 missed variants. The remaining 2 variants were present at low frequency in the WGS data but were not detected by the variant analysis pipeline.

FIG. 21A is the diagnostic yield from prospective sequencing of 42 consecutive MDS patients with WGS. FIG. 21A is organized as in FIG. 7B. Consecutive MDS patients were sequenced from April 2019 to February 2020 to estimate the diagnostic yield of WGS compared to standard testing. Top panel shows the cumulative number of patients with new findings (in blue) and the cumulative number of patients with findings that changed the IPSS-R cytogenetic risk score. IPSS-R from cytogenetics and sequencing are shown below.

FIG. 21B shows chromosomal abnormalities obtained from WGS and indicates new findings (in blue) and findings that changed the IPSS-R category (red) in top panel. Bottom panel shows mutations in MDS-associated genes referenced in NCCN guidelines, with concordance between WGS and targeted sequencing indicated by the color (concordant in black, WGS only in orange, and targeted sequencing only in green). Note that the yield in risk-defining events in this cohort is due entirely to cases where cytogenetics was unsuccessful or inconclusive, resulting in an undetermined IPSS-R risk category.

FIG. 22A represents AML patients with reclassified risk groups by WGS. AML patients included in the outcome analysis were with defined cytogenetic risk that were reclassified by WGS. It shows the ELN risk groups by cytogenetics combined with FISH for PML-RARA, RUNX1-RUNX1T1, CBFB-MYH11, del(5q), and del(7q) (bottom), and gene mutation testing either via targeted sequencing or PCR, and WGS and FLT3-ITD PCR only (top).

FIG. 22B show WGS results (with the risk-defining event highlighted in red), and results from cytogenetics, FISH, and gene mutation analysis for AML patients with reclassified risk groups by WGS. FLT3-ITD mutation status by PCR was used for both WGS and conventional risk group assignments. Cells outlined in red indicate that testing was either not performed or failed. Also shown is the clinical status of each patient, including whether they expired or relapsed, and the follow-up time in months from diagnosis. WGS identified new adverse risk findings in 5 patients, while 3 patients had differences in gene mutations in ASXL1 and NPM1 and either due to lack of testing at diagnosis (N=2) or a missed low abundance NPM1c mutation by WGS (N=1).

FIG. 23A is a survival analysis of 101 AML patients with defined risk. It shows Kaplan Meier survival curves using death as the endpoint for 101 AML patients treated with either post-remission chemotherapy alone (N=71) or allogeneic stem cell transplant (N=30) stratified by ELN risk groups from cytogenetics, targeted sequencing, and FLT3 ITD mutation testing. Log-rank test for equal survival across the groups, adjusted P=0.43. Age adjusted Cox regression for death in not adverse vs. adverse cytogenetic risk groups: 1.06, 95% CI 0.45 to 2.50.

FIG. 23B shows Kaplan Meier survival curves using death as the endpoint for 101 AML patients treated with either post-remission chemotherapy alone (N=71) or allogeneic stem cell transplant (N=30) stratified by ELN risk groups from WGS and FLT3 ITD mutation testing. Log-rank test for equal survival across the groups, adjusted P=0.09. Age-adjusted Cox regression for death in not adverse and adverse WGS-based risk groups: 0.59, 95% CI 0.26 to 1.36.

FIG. 24A are WGS results for AML patients with inconclusive cytogenetics, or patients with unsuccessful (N=6), inconclusive (N=13), or unknown (N=8) cytogenetic and FISH studies that precluded definitive genomic risk assignment. WGS-based ELN risk group is shown in the top panel.

FIG. 24B are results from WGS, cytogenetics, FISH, and gene mutation testing. FLT3-ITD mutation status was determined by PCR and was used for risk stratification according to ELN criteria using an allele ratio cutoff of 0.5, or presence/absence if an allele ratio was not available. Bottom panel shows clinical status and follow-up time in months. WGS identified risk-defining chromosomal abnormalities in four patients, including KMT2A and RUNX1-RUNX1T1 rearrangements (N=1 each) and a complex karyotype (N=2). The remaining 23 patients were assigned to ELN risk groups based on gene mutations.

FIG. 25A is a survival analysis of AML patients with inconclusive cytogenetics, showing a Kaplan Meier survival curve for 27 AML patients with unsuccessful for inconclusive cytogenetics stratified by gene mutations only. Patients were considered intermediate risk unless favorable risk or adverse risk gene mutations were identified. Log-rank test for equal survival across the groups, adjusted P=0.09. Age-adjusted Cox regression hazard ratio for death in not adverse vs. adverse risk, 0.4; 95% CI, 0.14 to 1.1.

FIG. 25B is a Kaplan Meier survival curve using death as the endpoint for the above cohort of 38 patients stratified by ELN risk groups from WGS and FLT3 ITD mutation testing. Median survival for this expanded cohort was 22.3 months, 95% CI, 6.8 to 46.1. Log-rank test for equal survival across the groups, adjusted P=0.02. Age-adjusted Cox regression hazard ratio for death in not adverse vs. adverse risk, 0.28; 95% CI, 0.11 to 0.71.

There are shown in the drawings arrangements that are presently discussed, it being understood, however, that the present embodiments are not limited to the precise arrangements and are instrumentalities shown. While multiple embodiments are disclosed, still other embodiments of the present disclosure will become apparent to those skilled in the art from the following detailed description, which shows and describes illustrative aspects of the disclosure. As will be realized, the invention is capable of modifications in various aspects, all without departing from the spirit and scope of the present disclosure. Accordingly, the drawings and detailed description are to be regarded as illustrative in nature and not restrictive.

### DETAILED DESCRIPTION

In various aspects, a rapid method based on whole-genome sequencing that recapitulates and improves upon conventional cytogenetics is disclosed. Whole-genome sequencing (WGS) can detect clinically relevant chromosomal rearrangements with unparalleled accuracy and would vastly improve karyotyping of tumors by making it possible to test nearly any tumor type (including formalin-fixed specimens) for virtually all clinically-relevant chromosomal rearrangements simultaneously. The methods disclosed herein advance 50-year-old cytogenetic methods to the modern era by greatly expanding the range of mutations that can be detected and tumor types that can be analyzed with the ultimate goal of improving clinical decisions and patient outcomes. The disclosed method includes both software and wet laboratory workflows.

Genetic profiling is a routine component of the diagnostic workup for an increasing number of cancers and is used to predict clinical outcomes and responses to targeted therapies. Mutations that are clinically actionable for any individual type of cancer typically span a wide range of genomic events, including chromosomal rearrangements, gene amplifications and deletions, and single-nucleotide changes. The diversity of these findings necessitates the use of multiple platforms to obtain the genetic information needed for clinical management. Whole-genome sequencing is an unbiased method of detecting all types of mutations and could potentially be used to replace current testing algorithms. Such sequencing can also be performed on a limited amount of DNA and can identify genomic changes that may be cryptic in other types of analyses. These features of whole-genome sequencing make the methods disclosed herein suitable for genomic profiling in patients with cancer.

At least several features of WGS make it particularly well-suited for use in clinical testing. WGS can be performed using minimal amounts of DNA (as little as 50 ng) and from any specimen type, including formalin-fixed solid tumor tissue obtained from routine surgical biopsies, and so can be applied to nearly any tumor type and is not limited to only those tumor types with live cells. WGS procedures also do not involve complicated and time-consuming laboratory steps that are required for other sequencing methods, including complex library preparation procedures, hybridization-capture enrichment, or intact RNA, making it one of the most rapid next-generation sequencing assays and among the simplest to implement in a clinical laboratory. Finally, because WGS produces genome-wide, base-pair resolution genomic data, it can be analyzed for chromosomal rearrangements and gene-level mutations simultaneously, thereby providing a comprehensive profile of all clinically relevant mutations regardless of mutation type.

In various aspects, the disclosed method includes providing or obtaining a biological sample comprising tumor DNA. Any suitable biological sample may be used in the disclosed method without limitation including, but not limited to, peripheral blood, bone marrow aspirate, solid tumor biopsy samples, and any other suitable biological sample. The sample size may be at least about 200 μL for samples such as peripheral blood and bone marrow aspirate.

To reduce time, complexity, and cost, the disclosed method is implemented without a normal tissue comparator because the method is configured to identify clearly pathogenic somatic events that generally do not require a germline control. In some aspects, the biological sample is provided as previously-obtained WGS data. In other aspects, method includes extracting the tumor DNA from the biological sample using any suitable method without limitation including, but not limited to, the QIAamp DNA mini kit (Qiagen, Hilden, Germany) as detailed in the package insert, followed by quantification with the Qubit 1.0 fluorometer High Sensitivity dsDNA assay (ThermoFisher, Waltham, Mass.) as described in the Example below.

In various aspects, the method further includes performing library preparation using the tumor DNA extracted from the biological sample. In some aspects, the amount of tumor DNA used for library preparation ranges from about 35 ng to about 500 ng or more. Any suitable method of library preparation may be used without limitation including, but not limited to the Nextera Flex library preparation kit (cat #20015804, Illumina, Inc, San Diego, Calif.) as described in the Examples below. In some aspects, the quantified final libraries are diluted to 1 nM for equimolar pooling prior to sequencing

In various aspects, the method further includes subjecting the library to whole-genome sequencing using any suitable systems and associated methods without limitation. In one exemplary aspect, WGS is performed using a NovaSeq 6000 sequencing instrument (Illumina) configured to obtain about 60× genome coverage per sample. In various other aspects, the WGS may be configured to obtain a genome coverage ranging from about 10× per sample to about 100× per sample. In other aspects, the WGS may be configured to obtain a genome coverage of about 10×, 20×, 30×, 40×, 50×, 60×, 70×, 80×, 90×, 100×, or more per sample.

In various aspects, the method further includes aligning the WGS data to a human reference genome including, but not limited to the GRCh38 human reference genome. Any suitable alignment method may be used without limitation including local alignment software such as DRAGEN (version 3.5.7) hardware-accelerated sequence processing software suite or cloud-based alignment software such as the DRAGEN Germline (alignment only) BaseSpace App. In various aspects, the alignments are provided for variant analysis in any suitable format including, but not limited to CRAM format.

In various aspects, the disclosed method makes use of a streamlined approach for rapid whole-genome sequencing and analysis that detects clinically relevant cytogenetic abnormalities in a range of tumor types. In various aspects, the ChromoSeq WGS assay is a high-performance tumor-only WGS analysis pipeline that generates a digital karyotype and detects known chromosomal rearrangements and gene-level mutations based on analysis of raw WGS sequence data produced from a single biological sample.

In various aspects, the method further includes performing variant analysis on the alignments using the ChromoSeq WGS assay. Variant analysis includes CNA identification, SV identification, and gene-level variant identification. In some aspects, each portion of the variant analysis is limited to targeted analysis and filtering to streamline the process while yielding variants that are be clinically relevant. In various aspects, each portion of the variant analysis is performed using the same alignments from the single biological sample.

For CNA identification, the alignments are transformed into read counts in 500,000 bp nonoverlapping windows across the genome and the read counts are transformed into CNAs using any suitable method including, but not limited to, the purity and subclone-aware Hidden Markov Model, ichorCNA as described in the examples below. In some aspects, the reported CNAs are filtered to remove CNAs <5 Mbp; cytogenetically evident CNAs are typically greater than plurality of CNAs, a size potentially detectable by karyotype analysis.

For SV identification, a break-end caller Manta is used to detect SV calls of at least 100 kbp in length to reduce the number of calls with unknown clinical significance. The detected SVs are then filtered to identify translocations, deletions, duplications, and inversions that overlap a curated list of 612 recurrent and/or risk-defining SVs obtained from published sources, including the WHO and the Atlas of Genetics and Cytogenetics in Oncology. A list of the recurrent and/or risk-defining SVs is provided as Table S2 in the examples below. Genomic events where both ends overlap one of these recurrent SVs are reported as ‘top-level’ findings in ChromoSeq without additional filtering. The remaining SVs are subsequently filtered to remove patient-specific events and/or identify cytogenetically cryptic rearrangements involving genes relevant for AML or MDS. In some aspects, the filtering criteria may include retaining those SVs based on: 1) at least 2 ‘paired and 2 ‘split’ reads supporting the break-ends, 2) absence of an overlapping call from a large set of SVs identified from >17,795 human genomes, 3) coverage depth of deletion or duplication call must be <0.8 or >1.3 compared to the background, respectively, and 4) a defined breakpoint must be identified and the spanning contig generated by Manta must map back to the reported breakpoints.

Gene-level variants are identified using the same alignments from the biological sample as used for CNA and SV analysis. In some aspects, gene mutations are identified within about 85 kbp targeting 40 genes and gene hotspots that are recurrently mutated in AML or MDS. In other aspects, an indel caller is run on exons 13-15 of FLT3 to identify FLT3 ITD alleles.

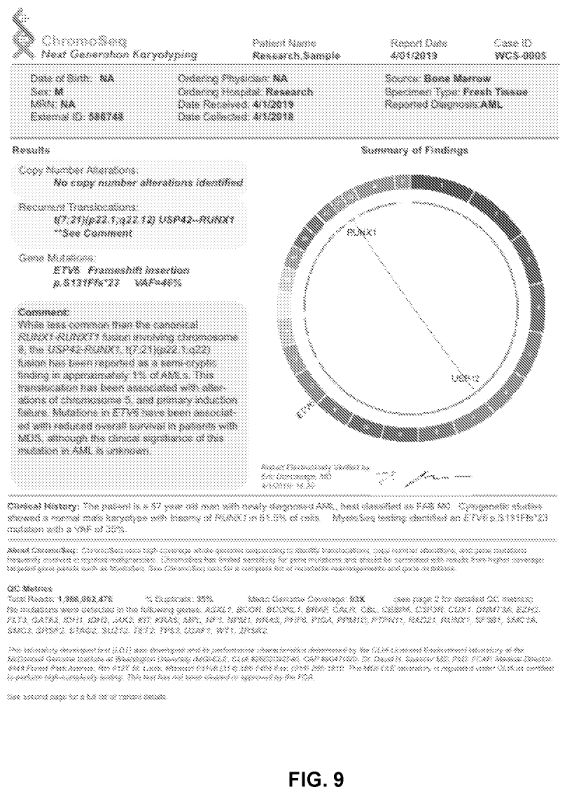

In various aspects, the method includes combining the annotated CNA, SV, and gene mutation calls obtained as described above are combined with coverage QC information to generate a final text report as well as data files (CRAM, and VCF) and graphical coverage plots from ichorCNA. In some aspects, a graphical report is generated, as shown in FIG. 9.

In various aspects, the disclosed method detects clinically significant structural variants (copy number alterations and translocations) from WGS data, and provides the ability to replicate and outperform conventional cytogenetic testing. The ChromoSeq WGS automated workflow sifts down up to 10,000 or more potential CAN and SV calls to 1 or 2 real events without sequencing paired normal tissue, greatly streamlining and simplifying WGS for use as a clinical assay.

In various aspects, the disclosed methods are streamlined at each phase to provide for clinically timely results. In some aspects, scalable methods of sample preparation that can be performed by a single technician in less than 8 hours with commercially available reagents are used. In other aspects, the samples are subjected to high-throughput sequencing followed by automated tumor-only variant analysis to detect mutations in selected genes, copy-number alterations of more than 5 Mbp, and recurrent structural variants. In additional aspects, the method includes automatically generating the findings of the analysis in a concise clinical report.

In addition to improving on existing clinical methods such as conventional cytogenetics, the methods disclosed herein obviate at least a portion of the challenges associated with the use of WGS for clinical testing, in particular the high cost of sequencing and the complex analysis of results that are typically too complicated and time consuming for clinical laboratories. In some aspects, the disclosed methods include a simplified, tumor-only WGS analysis strategy that focuses on detecting known, clinically-relevant chromosome-level mutations that are routinely tested for by clinical cytogenetic laboratories. By limiting WGS analysis to mutations with established clinical relevance, the disclosed method greatly reduces the time, cost, and complexity of WGS, while also expanding the number of mutations that can be queried in each sample. Previous research of tumors using WGS have been focused on comprehensive discovery of new mutations, rather than efficient and comprehensive detection of known mutations.

In other aspects, the disclosed method makes use of recently-developed sequencing systems including, but not limited to, the Illumina NovaSeq 6000 instrument to perform WGS of tumor samples. Such systems are capable of generating high coverage WGS data in a matter of days at a cost that is comparable to standard karyotyping and cytogenetic analysis that is typically used for clinical testing.

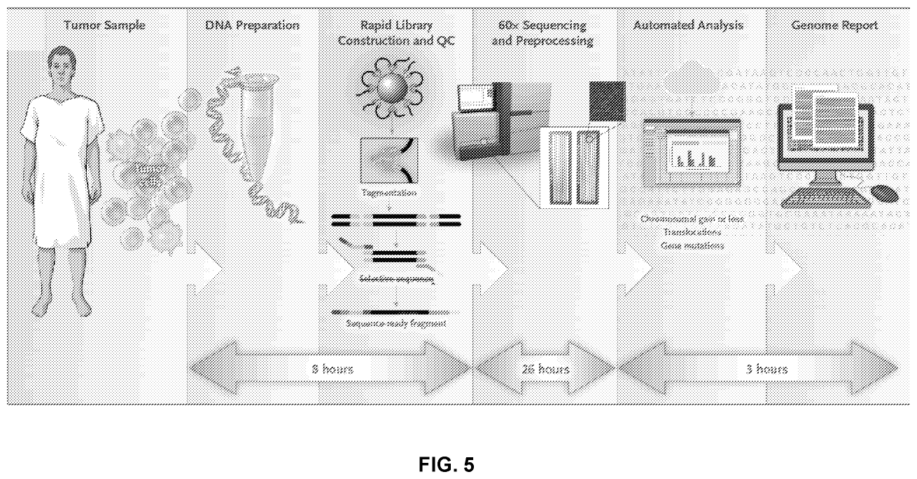

In various aspects, a streamlined approach to whole-genome sequencing (ChromoSeq) is disclosed. The disclosed method is designed to provide comprehensive genomic profiling of clinically relevant mutations in samples obtained from patients with AML or MDS, while minimizing the turnaround time and technical complexity. An overview of the disclosed method is provided in FIG. 5. As illustrated in FIG. 5, scalable methods of sample preparation that can be performed by a single technician in less than 8 hours with commercially available reagents were used, followed by standard high-throughput sequencing. Automated tumor-only variant analysis detected mutations in selected genes, copy-number alterations of more than 5 Mbp, and recurrent structural variants (Tables S1 and S2). The findings of the WGS analysis is summarized in a concise clinical report to a practitioner (FIG. 9).

As described in the Examples below, that whole-genome sequencing provided rapid and accurate genomic profiling in patients with AML or MDS. WGS sequencing also provided a greater diagnostic yield than conventional cytogenetic analysis and more efficient risk stratification on the basis of standard risk categories.

Genomic abnormalities are particularly important for diagnostic classification and risk assessment in patients with acute myeloid leukemia (AML) and myelodysplastic syndromes (MDS). Recurrent chromosomal abnormalities are the basis for the AML genomic classification system of the World Health Organization, and the association of these alterations and certain genetic mutations with clinical outcomes has led to the development of algorithms for genetic risk stratification in patients with AML. Similar studies involving patients with MDS have resulted in the cytogenetic component of the International Prognostic Scoring System-Revised (IPSS-R) in such patients. Although advances in sequencing technology have improved the ability to identify genetic mutations, the detection of chromosomal rearrangements is primarily performed through conventional metaphase cytogenetic analysis (i.e., karyotyping). The latter approach is effective but has several limitations, including the need to obtain viable cells, low sensitivity, and limited resolution. Fluorescence in situ hybridization (FISH) and targeted sequencing assays that use DNA, RNA, or both are also used, but these methods are informative only in the regions selected for analysis and may provide incomplete information regarding identified chromosomal rearrangements. As a result, conventional cytogenetic analysis remains an essential component of the diagnostic workup for patients with AML or MDS.

In various aspects, the method includes the use of whole-genome sequencing in place of standard testing for genetic profiling in AML and MDS patients. Although the high cost of sequencing and complex, time-consuming analysis methods have historically restricted such sequencing to research studies, recent advances have made this analysis simpler to perform, faster, and less expensive. As described in the Examples below, the method includes a streamlined approach to whole-genome sequencing for genomic profiling of patients with AML or MDS.

As described in the examples below, the clinical utility of whole-genome sequencing for the genomic evaluation of patients with AML or MDS was demonstrated. Results from 263 patients showed that such sequencing was equivalent to or better than conventional testing, both in analytical performance and clinical applicability. Whole-genome sequencing detected 100% of the clinically significant abnormalities that had been identified by the existing clinical methods, cytogenetic analysis and clinical FISH assays. In addition, whole-genome sequencing provided new genetic information in 25% of patients, more than half of whom would have been assigned to a different genetic risk category with results from conventional testing.

In some aspects, the diagnostic yield of whole-genome sequencing will depend on laboratory-specific karyotyping practices and the use of FISH or other ancillary testing; and some rapid diagnostic assays may still be used for urgent treatment decisions (e.g., FISH or quantitative PCR for PML-RARA rearrangements and PCR for FLT3-ITD mutations). However, the Examples below demonstrate that whole-genome sequencing provides definitive results for clinically relevant genomic events with the use of a single test.

Prospective real-time sequencing of samples obtained from consecutive patients, described below, showed that whole-genome sequencing yields complete genomic information in a clinically relevant timeframe. This speed resulted from faster laboratory methods and automated data analysis that focused on clinically relevant mutations, which allowed the generation of reports in as little as 3 days. In some aspects, WGS results are suitable for use in risk predictions with existing, clinically validated risk-stratification systems. The disclosed method adds prognostic value by expanding risk stratification to more patients, especially for those with inconclusive results on cytogenetic analysis, where whole-genome sequencing could have an immediate effect on treatment decisions.

Implementing whole-genome sequencing for clinical testing can provide a unified, stable, and extensible platform that minimizes laboratory-specific bias and that can be standardized throughout the world. Although the disclosed method is described herein for use in diagnosing myeloid cancers, many of the advantages of whole-genome sequencing directly apply to patients with other cancers. Whole-genome sequencing can be performed on DNA from tissue biopsy samples of solid tumors, which are often insufficient for standard molecular assays and difficult to culture for cytogenetic studies. The benefits of WGS may be even greater for these cancer types, in which whole-genome sequencing could be used to rapidly survey the entire genome for an expanding number of key mutations and structural alterations with only a small amount of DNA. Such an approach would simplify genomic testing for these patients and probably increase the yield of clinically relevant findings, which improve the precision of approaches for treating many patients with cancer.

In various aspects, at least a portion of the disclosed whole-genome sequencing methods may be implemented using various computing systems and devices as described below.

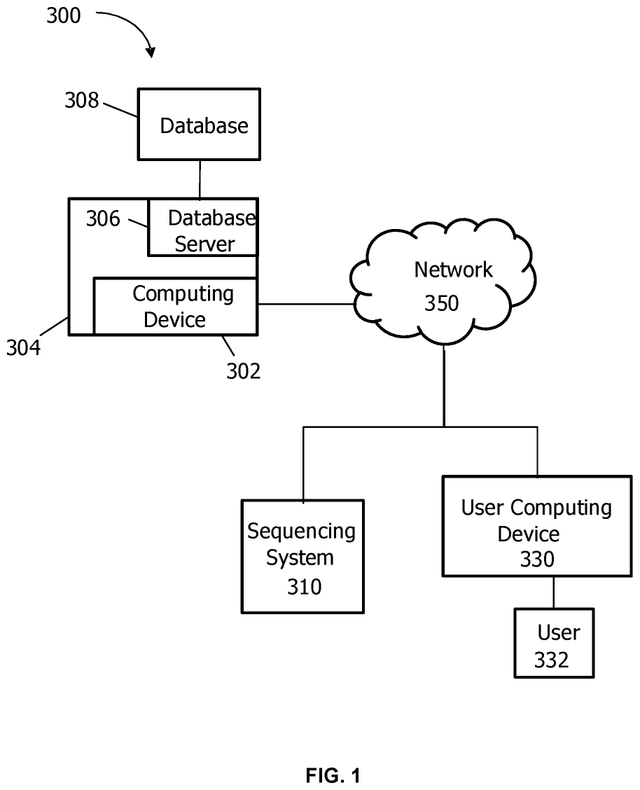

FIG. 1 depicts a simplified block diagram of a computing device for implementing the methods described herein. As illustrated in FIG. 1, the computing device 300 may be configured to implement at least a portion of the tasks associated with the disclosed method using a whole-genome sequencing system 310 including, but not limited to: operating the sequencing system 310 to obtain whole-genome sequencing (WGS) data, analyzing the WGS data to identify mutations, copy-number alterations, structural variants, and generating a clinical report of findings. The computer system 300 may include a computing device 302. In one aspect, the computing device 302 is part of a server system 304, which also includes a database server 306. The computing device 302 is in communication with a database 308 through the database server 306. The computing device 302 is communicably coupled to the sequencing system 310 and a user-computing device 330 through a network 350. The network 350 may be any network that allows local area or wide area communication between the devices. For example, the network 350 may allow communicative coupling to the Internet through at least one of many interfaces including, but not limited to, at least one of a network, such as the Internet, a local area network (LAN), a wide area network (WAN), an integrated services digital network (ISDN), a dial-up-connection, a digital subscriber line (DSL), a cellular phone connection, and a cable modem. The user-computing device 330 may be any device capable of accessing the Internet including, but not limited to, a desktop computer, a laptop computer, a personal digital assistant (PDA), a cellular phone, a smartphone, a tablet, a phablet, wearable electronics, smartwatch, or other web-based connectable equipment or mobile devices.

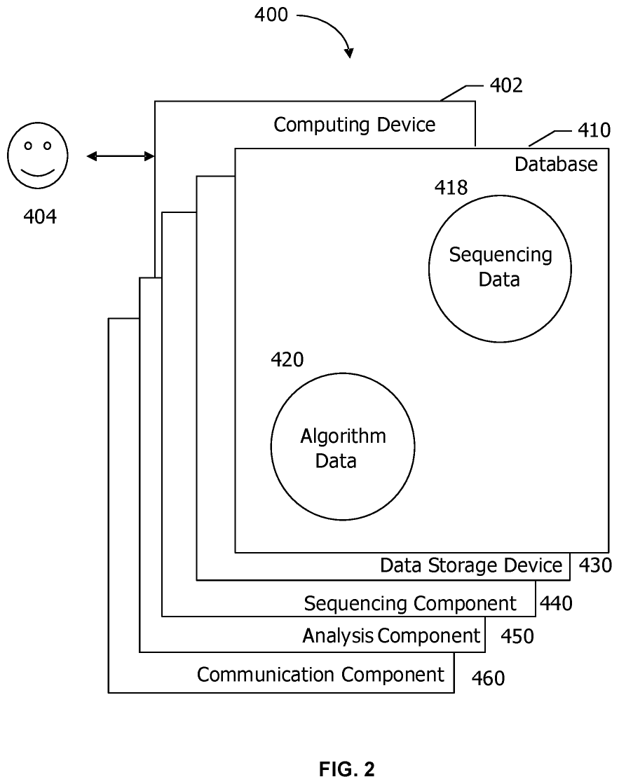

In other aspects, the computing device 302 is configured to perform a plurality of tasks associated with the disclosed whole-genome sequencing method. FIG. 2 depicts a component configuration 400 of computing device 402, which includes database 410 along with other related computing components. In some aspects, computing device 402 is similar to computing device 302 (shown in FIG. 1). A user 404 may access components of computing device 402. In some aspects, database 410 is similar to database 308 (shown in FIG. 1).

In one aspect, database 410 includes sequencing data 418 and algorithm data 420. Non-limiting examples of suitable sequencing data 418 include any data associated with the whole genome sequencing and alignment. Non-limiting examples of suitable algorithm data 420 include any values of parameters defining the analysis of the whole genome sequencing data, such as any of the parameters defining the WGS library, variant analysis, copy-number alteration identification, and structural variant analysis. Other non-limiting examples of suitable algorithm data 420 include any parameters defining the formatting of a clinical report of results.

Computing device 402 also includes a number of components that perform specific tasks. In the exemplary aspect, computing device 402 includes a data storage device 430, segmentation component 440, analysis component 450, and communication component 460. Data storage device 430 is configured to store data received or generated by computing device 402, such as any of the data stored in database 410 or any outputs of processes implemented by any component of computing device 402. Sequencing component 440 is configured to operate or produce signals configured to operate, a sequencing system to obtain and align whole-genome sequencing data. Analysis component 450 is configured to analyze the WGS data and generate clinical reports as described herein.

Communication component 460 is configured to enable communications between computing device 402 and other devices (e.g. user computing device 330 and sequencing system 310, shown in FIG. 1) over a network, such as network 350 (shown in FIG. 1), or a plurality of network connections using predefined network protocols such as TCP/IP (Transmission Control Protocol/Internet Protocol).

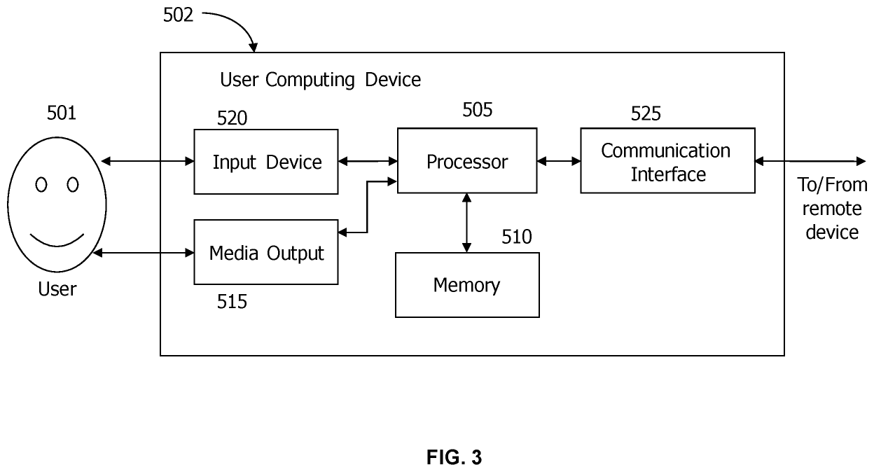

FIG. 3 depicts a configuration of a remote or user-computing device 502, such as user computing device 330 (shown in FIG. 1). Computing device 502 may include a processor 505 for executing instructions. In some aspects, executable instructions may be stored in a memory area 510. Processor 505 may include one or more processing units (e.g., in a multi-core configuration). Memory area 510 may be any device allowing information such as executable instructions and/or other data to be stored and retrieved. Memory area 510 may include one or more computer-readable media.

Computing device 502 may also include at least one media output component 515 for presenting information to a user 501. Media output component 515 may be any component capable of conveying information to user 501. In some aspects, media output component 515 may include an output adapter, such as a video adapter and/or an audio adapter. An output adapter may be operatively coupled to processor 505 and operatively coupleable to an output device such as a display device (e.g., a liquid crystal display (LCD), organic light emitting diode (OLED) display, cathode ray tube (CRT), or “electronic ink” display) or an audio output device (e.g., a speaker or headphones). In some aspects, media output component 515 may be configured to present an interactive user interface (e.g., a web browser or client application) to user 501.

In some aspects, computing device 502 may include an input device 520 for receiving input from user 501. Input device 520 may include, for example, a keyboard, a pointing device, a mouse, a stylus, a touch-sensitive panel (e.g., a touchpad or a touch screen), a camera, a gyroscope, an accelerometer, a position detector, and/or an audio input device. A single component such as a touch screen may function as both an output device of media output component 515 and input device 520.

Computing device 502 may also include a communication interface 525, which may be communicatively coupleable to a remote device. Communication interface 525 may include, for example, a wired or wireless network adapter or a wireless data transceiver for use with a mobile phone network (e.g., Global System for Mobile communications (GSM), 3G, 4G or Bluetooth) or other mobile data network (e.g., Worldwide Interoperability for Microwave Access (WIMAX)).

Stored in memory area 510 are, for example, computer-readable instructions for providing a user interface to user 501 via media output component 515 and, optionally, receiving and processing input from input device 520. A user interface may include, among other possibilities, a web browser and client application. Web browsers enable users 501 to display and interact with media and other information typically embedded on a web page or a website from a web server. A client application allows users 501 to interact with a server application associated with, for example, a vendor or business.

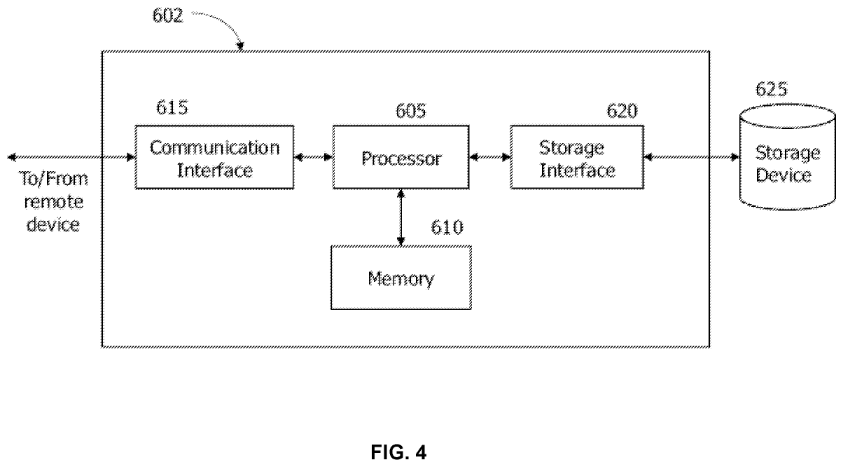

FIG. 4 illustrates an example configuration of a server system 602. Server system 602 may include, but is not limited to, database server 306 and computing device 302 (both shown in FIG. 1). In some aspects, server system 602 is similar to server system 304 (shown in FIG. 1). Server system 602 may include a processor 605 for executing instructions. Instructions may be stored in a memory area 625, for example. Processor 605 may include one or more processing units (e.g., in a multi-core configuration).

Processor 605 may be operatively coupled to a communication interface 615 such that server system 602 may be capable of communicating with a remote device such as user computing device 330 (shown in FIG. 1) or another server system 602. For example, communication interface 615 may receive requests from user computing device 330 via a network 350 (shown in FIG. 1).

Processor 605 may also be operatively coupled to a storage device 625. Storage device 625 may be any computer-operated hardware suitable for storing and/or retrieving data. In some aspects, storage device 625 may be integrated in server system 602. For example, server system 602 may include one or more hard disk drives as storage device 625. In other aspects, storage device 625 may be external to server system 602 and may be accessed by a plurality of server systems 602. For example, storage device 625 may include multiple storage units such as hard disks or solid-state disks in a redundant array of inexpensive disks (RAID) configuration. Storage device 625 may include a storage area network (SAN) and/or a network attached storage (NAS) system.

In some aspects, processor 605 may be operatively coupled to storage device 625 via a storage interface 620. Storage interface 620 may be any component capable of providing processor 605 with access to storage device 625. Storage interface 620 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 605 with access to storage device 625.

Memory areas 510 (shown in FIG. 3) and 610 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are example only, and are thus not limiting as to the types of memory usable for storage of a computer program.

The computer systems and computer-implemented methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local or remote processors, transceivers, servers, and/or sensors (such as processors, transceivers, servers, and/or sensors mounted on vehicle or mobile devices, or associated with smart infrastructure or remote servers), and/or via computer executable instructions stored on non-transitory computer-readable media or medium.

In some aspects, a computing device is configured to implement machine learning, such that the computing device “learns” to analyze, organize, and/or process data without being explicitly programmed. Machine learning may be implemented through machine learning (ML) methods and algorithms. In one aspect, a machine learning (ML) module is configured to implement ML methods and algorithms. In some aspects, ML methods and algorithms are applied to data inputs and generate machine learning (ML) outputs. Data inputs may further include: sequencing data, sensor data, image data, video data, telematics data, authentication data, authorization data, security data, mobile device data, geolocation information, transaction data, personal identification data, financial data, usage data, weather pattern data, “big data” sets, and/or user preference data. In some aspects, data inputs may include certain ML outputs.

In some aspects, at least one of a plurality of ML methods and algorithms may be applied, which may include but are not limited to: linear or logistic regression, instance-based algorithms, regularization algorithms, decision trees, Bayesian networks, cluster analysis, association rule learning, artificial neural networks, deep learning, dimensionality reduction, and support vector machines. In various aspects, the implemented ML methods and algorithms are directed toward at least one of a plurality of categorizations of machine learning, such as supervised learning, unsupervised learning, and reinforcement learning.

In one aspect, ML methods and algorithms are directed toward supervised learning, which involves identifying patterns in existing data to make predictions about subsequently received data. Specifically, ML methods and algorithms directed toward supervised learning are “trained” through training data, which includes example inputs and associated example outputs. Based on the training data, the ML methods and algorithms may generate a predictive function that maps outputs to inputs and utilize the predictive function to generate ML outputs based on data inputs. The example inputs and example outputs of the training data may include any of the data inputs or ML outputs described above.

In another aspect, ML methods and algorithms are directed toward unsupervised learning, which involves finding meaningful relationships in unorganized data. Unlike supervised learning, unsupervised learning does not involve user-initiated training based on example inputs with associated outputs. Rather, in unsupervised learning, unlabeled data, which may be any combination of data inputs and/or ML outputs as described above, is organized according to an algorithm-determined relationship.

In yet another aspect, ML methods and algorithms are directed toward reinforcement learning, which involves optimizing outputs based on feedback from a reward signal. Specifically ML methods and algorithms directed toward reinforcement learning may receive a user-defined reward signal definition, receive a data input, utilize a decision-making model to generate an ML output based on the data input, receive a reward signal based on the reward signal definition and the ML output, and alter the decision-making model so as to receive a stronger reward signal for subsequently generated ML outputs. The reward signal definition may be based on any of the data inputs or ML outputs described above. In one aspect, an ML module implements reinforcement learning in a user recommendation application. The ML module may utilize a decision-making model to generate a ranked list of options based on user information received from the user and may further receive selection data based on a user selection of one of the ranked options. A reward signal may be generated based on comparing the selection data to the ranking of the selected option. The ML module may update the decision-making model such that subsequently generated rankings more accurately predict a user selection.

As will be appreciated based upon the foregoing specification, the above-described aspects of the disclosure may be implemented using computer programming or engineering techniques including computer software, firmware, hardware or any combination or subset thereof. Any such resulting program, having computer-readable code means, may be embodied or provided within one or more computer-readable media, thereby making a computer program product, i.e., an article of manufacture, according to the discussed aspects of the disclosure. The computer-readable media may be, for example, but is not limited to, a fixed (hard) drive, diskette, optical disk, magnetic tape, semiconductor memory such as read-only memory (ROM), and/or any transmitting/receiving medium, such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor may include any programmable system including systems using micro-controllers, reduced instruction set circuits (RISC), application specific integrated circuits (ASICs), logic circuits, and any other circuit or processor capable of executing the functions described herein. The above examples are examples only, and are thus not intended to limit in any way the definition and/or meaning of the term “processor.”

As used herein, the terms “software” and “firmware” are interchangeable, and include any computer program stored in memory for execution by a processor, including RAM memory, ROM memory, EPROM memory, EEPROM memory, and non-volatile RAM (NVRAM) memory. The above memory types are example only, and are thus not limiting as to the types of memory usable for storage of a computer program.

In one aspect, a computer program is provided, and the program is embodied on a computer-readable medium. In one aspect, the system is executed on a single computer system, without requiring a connection to a server computer. In a further aspect, the system is being run in a Windows® environment (Windows is a registered trademark of Microsoft Corporation, Redmond, Wash.). In yet another aspect, the system is run on a mainframe environment and a UNIX® server environment (UNIX is a registered trademark of X/Open Company Limited located in Reading, Berkshire, United Kingdom). The application is flexible and designed to run in various different environments without compromising any major functionality.

In some aspects, the system includes multiple components distributed among a plurality of computing devices. One or more components may be in the form of computer-executable instructions embodied in a computer-readable medium. The systems and processes are not limited to the specific aspects described herein. In addition, components of each system and each process can be practiced independent and separate from other components and processes described herein. Each component and process can also be used in combination with other assembly packages and processes. The present aspects may enhance the functionality and functioning of computers and/or computer systems.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Any publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following examples illustrate various aspects of the disclosure.

### Example 1: Genome Sequencing as an Alternative to Cytogenetic Analysis in Myeloid Cancers

To develop and validate a whole-genome sequencing method for detecting genomic profiling in patients with acute myeloid leukemia (AML) or myelodysplastic syndromes (MDS), the following experiments were conducted.

Patients

All the samples that were included in this study were obtained from patients with a known or suspected diagnosis of AML or MDS. All the patients provided written informed consent for genomic sequencing studies. Samples were selected for sequencing for three specific purposes: 1) WGS performance assessment, 2) Establishing diagnostic yield and clinical feasibility, and 3) Evaluation of risk prediction in patients with unsuccessful or incomplete cytogenetic studies. To achieve these objectives, a combination of retrospective and prospective patient cohorts was used as described below. Retrospective samples were obtained from cryopreserved diagnostic bone marrow or peripheral-blood specimens. Prospective samples were obtained from fresh bone marrow aspirate or peripheral-blood specimens collected from consecutive, unselected patients for whom clinical cytogenetic analysis by means of karyotyping had been requested.

Retrospective samples from AML and MDS patients (N=146) included DNA extracted from either cryopreserved bone marrow (N=133) or peripheral blood (N=13) specimens. For WGS performance evaluation, 111 samples were selected based on DNA availability and results from conventional cytogenetic studies in order to include a wide range of chromosomal abnormalities, including risk-defining translocations, copy number alterations (CNAs), and either a complex or normal karyotype. Separately, to determine whether WGS could be used to predict outcomes for patients with unknown cytogenetics, 35 retrospective samples were selected from patients treated with induction chemotherapy and for whom cytogenetics was unknown, unsuccessful, or inconclusive at diagnosis. Of note, samples with successful cytogenetic studies from the prospective cohort below were also used for WGS performance evaluation, and likewise, prospective samples with unsuccessful or inconclusive cytogenetics were used for the analysis of WGS-based risk prediction in patients with unknown cytogenetics.

Evaluation of the feasibility and diagnostic yield of WGS compared to standard testing used samples from a cohort of 117 prospective patients. These samples included bone marrow aspirate (N=116) or peripheral blood (N=1) specimens from 117 consecutive, unselected patients for whom clinical cytogenetic analysis via karyotyping was requested. The only selection criteria for these patients were patient consent and that there was sufficient remaining specimen left after standard cytogenetic analysis to be used for sequencing; some samples required the addition of RPMI based media to wash residual material out of the sodium heparin tubes prior to DNA extraction. This cohort included patients with both successful and unsuccessful cytogenetic studies, and therefore contributed to WGS performance evaluation and WGS-based risk prediction analysis for patients with unknown cytogenetics.

Whole Genome Sequencing

Tumor-only WGS was performed in a CLIA-licensed environment clinical sequencing laboratory; no normal tissue comparator was used for this assay in order to reduce time, complexity, and cost, and because the purpose is to identify clearly pathogenic somatic events. that generally do not require a germline control. All samples were accessioned into the MGI laboratory information management system (LIMS) upon receipt prior to DNA extraction (for prospective samples) or library preparation (for retrospective samples received as DNA). DNA from prospective peripheral blood or bone marrow aspirate specimens was extracted using 200 uL of material with the QIAamp DNA mini kit (Qiagen, Hilden, Germany) as detailed in the package insert, followed by quantification with the Qubit 1.0 fluorometer High Sensitivity dsDNA assay (ThermoFisher, Waltham, Mass.). Subsequent WGS procedures are described below.

We processed samples and performed sequencing to a target coverage depth of 60×. This analysis involved the identification of mutations in 40 genes, genomewide copy number alterations greater than 5 Mbp, and structural variants matching 612 recurrent structural alterations in myeloid cancers. Details regarding genetic identification and structural variants are provided in Tables 51 and S2 below. We used the results of whole-genome sequencing to assign patients to a genetic risk group through the same classification systems that are used for conventional analyses.

Library Preparation

WGS library preparation used the Nextera Flex library preparation kit (cat #20015804, Illumina, Inc, San Diego, Calif.) along with dual unique index library adapters (cat #20015881). This onbead tagmentation-based library construction method was selected because it is fast, simple, and automatable, and thus fits well in a clinical testing environment where training of laboratory staff and turnaround time are important considerations. For this study, library construction was performed in a single day in batches of 2 to 16 samples by individual laboratory staff who followed the protocol detailed in the package insert without modification. In general, 500 ng of input DNA was used for library construction, although as little as 35 ng was used when DNA amounts were limiting. Completed libraries were accessioned into the LIMS, then assessed for size using an Agilent 2100 Bioanalyzer with a DNA High Sensitivity chip (Agilent, Santa Clara, Calif.), and quantified via Qubit (ThermoFisher, Waltham, Mass.). Final libraries were optionally quantified further via qPCR (generally on a subsequent day) using Kapa SYBR Fast qPCR library quantification (Roche, Basel Switzerland), and then diluted to 1 nM for equimolar pooling prior to sequencing.

Sequencing

Sequencing was performed on NovaSeq 6000 sequencing instruments (Illumina) using either S1 or S4 flowcells and 2×150 sequencing chemistry. Retrospective samples were sequenced on S4 flowcells in pools of 16 (or pools of 4 samples on one S4 lane using the XP lane loader) and prospective ‘real-time’ sequencing used 51 flow cells in pools of 3 samples, which is designed to yield >133 Gbp of raw sequence and 60× genome coverage per sample. Flowcell loading and sequencing were performed as recommended by the manufacturer. Times for sequencing are 25 and 44 hours for 51 and S4 flowcells, respectively, and were documented in the MGI LIMS system.

Data Processing

Completed sequencing runs were processed into aligned CRAM files using the GRCh38 human reference genome via two approaches:
Local processing: For retrospective samples, instrument data were processed and demultiplexed into FASTQ files using bcl2fastq (Illumina) via the in-house LIMS. Data were then aligned via a local installation of the DRAGEN (version 3.5.7) hardware-accelerated sequence processing software suite using default alignment parameters. Processing time with this approach for S4 flowcells were generally 47 hours for sequencing data transfer and 12 hours for demultiplexing. Alignment times using DRAGEN ranged from 20-40 minutes depending on coverage.
BaseSpace processing: To speed the processing of prospective samples, data generated on S1 flowcells were streamed from the NovaSeq instrument to the cloud-based BaseSpace sequence analysis platform. This saved time at both the sequencing and data processing steps, owing to the shorter run time for S1 flowcells and ‘on-demand’ rapid data processing.

After the sequencing run completed, demultiplexing and FASTQ generation was automatically launched in BaseSpace. Data were then aligned via manual launching of the DRAGEN Germline (alignment only) BaseSpace App (version 3.2.8, see https://basespace.illumina.com/apps/6840834/DRAGEN-Germline-Pipeline), which completed in about the same amount of time as the local DRAGEN installation. We note that the manual step of launching DRAGEN can be automated via the BaseSpace API to further reduce turnaround time.

Alignments in CRAM format generated using both the in-house and cloud-based procedures were used as input for the variant analysis workflow described below.

Variant Analysis and Reporting

Tumor-only variant analysis used a custom analysis workflow (‘ChromoSeq’) specified in the WDL workflow language and executed using the Cromwell workflow engine1 in dockerized compute containers, which is available for public use as a custom application on BaseSpace (https://basespace.illumina.com/apps/6984978/Chromoseq—pending BaseSpace approval for public release). Analysis involves three components: CNA identification, SV identification, and gene-level variant identification. All three of these components are subject to targeted analysis and filtering to yield variants that may be clinically relevant.

CNA Identification

Cytogenetically evident CNAs greater than 5 Mbp, which are of a size potentially detectable by karyotype analysis, are identified via a read-depth approach using a previously published purity and subclone-aware Hidden Markov Model, ichorCNA2; https://github.com/GavinHaLab/ichorCNA). The input for this script is a file with read counts in 500,000 bp nonoverlapping windows across the genome, either generated using bedtools3 or outputted directly from the DRAGEN mapping software during alignment using the command: dragen -r Sobj->{dragenref}--fastq-list $fastqfile --fastq-list-sample-id $samplename --enable-cnv true --cnv-targetbed/staging/garza testing/reference/all sequences.fa.bed --cnv-interval-width 500000 --output-directory $cramout output-file-prefix $sample --output-format CRAM --enable-bam-indexing true --enable-duplicate-marking true. Binned read counts are supplied to ichorCNA for normalization for GC content, mappability, and using a ‘panel of normals’ normalization file generated from 20 normal karyotype cases per the instructions on the ichorCNA github repo. Outputs from ichorCNA were text-processed via a custom PERL script (available upon request) to retain CNAs >5 Mbp and converted to VCF format, and then combined with SV calls (below) for input into the ChromoSeq reporting script.

CNA abundance as a percentage was calculated using the equation:

Abundance=(2\hat{ }L2R−1.0)/((CN/2.0−1.0)))*100,

where L2R is the log 2 normalized coverage ratio vs. a panel of normals and CN is the estimated copy number for the event.

SV Identification

SV identification is performed with the break-end caller Manta and broken into two ‘tiers’. In the first tier, recurrrent and risk defining events are detected with a high sensitivity approach, and in the second tier, novel SVs are subject to more rigorous filtering. Manta is run directly from the aligned CRAM file in ‘tumor’ mode and with custom parameters to increase the sensitivity and limit calls to those that are at least 100 kbp in length to reduce the number of calls with unknown clinical significance. SVs are then filtered to identify translocations, deletions, duplications, and inversions that overlap a curated list of 612 recurrent and/or risk-defining SVs obtained from published sources, including the WHO and the Atlas of Genetics and Cytogenetics in Oncology (see Table S2). Genomic events where both ends overlap one of these recurrent SVs are reported as ‘top-level’ findings in ChromoSeq without additional filtering. Although the remaining SVs will rarely be clinically relevant, they could include patient-specific events or identify cytogenetically cryptic rearrangements involving genes relevant for AML or MDS. We therefore perform rigorous annotation and filtering using a custom PERL script (available upon request) and report the remaining high-quality novel events as secondary findings. The following criteria must be met to yield a passing call: 1) at least 2 ‘paired and 2 ‘split’ reads supporting the break-ends, 2) absence of an overlapping call from a large set of SVs identified from >17,795 human genomes5, 3) coverage depth of deletion or duplication call must be <0.8 or >1.3 compared to the background6, respectively, and 4) a defined breakpoint must be identified and the spanning contig generated by Manta must map back to the reported breakpoints. This procedure dramatically reduces the number of reported calls. For example, the mean number of raw Manta calls per case is >5,000; after filtering we reported a mean of 11 calls across all 263 cases in this study (including recurrent SVs). SVs are then converted to VCF format, combined with CAN calls (from above), and annotated with VEP7 using Ensembl version 90, prior to reporting with the ChromoSeq reporting script.

TABLE S2
Recurrent SVs in AML and MDS

| Chrom1 | | | Chrom2 | | | Gene_Pair | | |
|---|---|---|---|---|---|---|---|---|
| | Start | End | | Start | End | | Gene1 Strand | Gene2 Strand |
| chr1 | 3069210 | 3438621 | chr1 | 2228694 | 2310119 | PRDM16_SKI | + | + |
| chr1 | 110338505 | 110346677 | chr22 | 40410280 | 40636685 | RBM15_MRTFA | + | − |
| chr1 | 148808465 | 149032955 | chr5 | 150113836 | 150155860 | PDE4DIP_PDGFRB | + | − |
| chr1 | 154157317 | 154192058 | chr5 | 150113836 | 150155860 | TPM3_PDGFRB | − | − |
| chr1 | 186311651 | 186375325 | chr8 | 38411138 | 38468834 | TPR_FGFR1 | − | − |
| chr1 | 221701423 | 221742176 | chr1 | 3069210 | 3438621 | DUSP10_PRDM16 | − | + |
| chr1 | 234604268 | 234609525 | chr17 | 40309193 | 40356796 | IRF2BP2_RARA | − | + |
| chr10 | 134464 | 254626 | chr17 | 51177424 | 51260066 | ZMYND11_MBTD1 | + | − |
| chr10 | 21524674 | 21743630 | chr9 | 41890313 | 42129250 | MLLT10_CNTNAP3B | + | − |
| chr10 | 21524674 | 21743630 | chrX | 41333347 | 41364472 | MLLT10_DDX3X | + | + |
| chr10 | 32009009 | 32056431 | chr4 | 54229096 | 54298247 | KIF5B_PDGFRA | − | + |
| chr10 | 59788762 | 59906656 | chr5 | 150113836 | 150155860 | CCDC6_PDGFRB | − | − |
| chr10 | 74824935 | 75032284 | chr16 | 3725053 | 3880726 | KAT6B_CREBBP | + | − |
| chr10 | 94501433 | 94602098 | chr8 | 2938098 | 4994972 | HELLS_CSMD1 | + | − |
| chr10 | 101130504 | 101137789 | chr7 | 142299010 | 142813287 | TLX1_TRB | + | + |
| chr10 | 102394109 | 102402529 | chr6 | 117288299 | 117425855 | NFKB2_ROS1 | + | − |
| chr11 | 925808 | 1012245 | chr14 | 52004802 | 52069228 | AP2A2_NID2 | + | − |
| chr11 | 3675009 | 3797792 | chr11 | 3675009 | 3797792 | NUP98_NUP98 | − | − |
| chr11 | 3675009 | 3797792 | chr11 | 108665024 | 108940930 | NUP98_DDX10 | − | + |
| chr11 | 3675009 | 3797792 | chr11 | 118436463 | 118526832 | NUP98_KMT2A | − | + |
| chr11 | 3675009 | 3797792 | chr12 | 280128 | 389454 | NUP98_KDM5A | − | − |
| chr11 | 3675009 | 3797792 | chr17 | 7235027 | 7239506 | NUP98_PHF23 | − | − |
| chr11 | 3675009 | 3797792 | chr2 | 176092890 | 176095938 | NUP98_HOXD13 | − | + |
| chr11 | 3675009 | 3797792 | chr2 | 176107285 | 176109588 | NUP98_HOXD11 | − | + |
| chr11 | 3675009 | 3797792 | chr20 | 41028817 | 41124487 | NUP98_TOP1 | − | + |
| chr11 | 3675009 | 3797792 | chr3 | 87259403 | 87276587 | NUP98_POU1F1 | − | − |
| chr11 | 3675009 | 3797792 | chr3 | 100401192 | 100456319 | NUP98_LNP1 | − | + |
| chr11 | 3675009 | 3797792 | chr5 | 177133078 | 177300210 | NUP98_NSD1 | − | + |
| chr11 | 3675009 | 3797792 | chr6 | 138773508 | 138793317 | NUP98_CCDC28A | − | + |
| chr11 | 3675009 | 3797792 | chr7 | 27162434 | 27165530 | NUP98_HOXA9 | − | − |
| chr11 | 3675009 | 3797792 | chr7 | 27181514 | 27185216 | NUP98_HOXA11 | − | − |
| chr11 | 3675009 | 3797792 | chr7 | 27194363 | 27200106 | NUP98_HOXA13 | − | − |
| chr11 | 3675009 | 3797792 | chr8 | 38269696 | 38382272 | NUP98_NSD3 | − | − |
| chr11 | 3675009 | 3797792 | chr9 | 15464065 | 15511019 | NUP98_PSIP1 | − | − |
| chr11 | 3675009 | 3797792 | chr9 | 129665640 | 129722674 | NUP98_PRRX2 | − | + |
| chr11 | 3675009 | 3797792 | chrX | 150983285 | 150990775 | NUP98_HMGB3 | − | + |
| chr11 | 3855701 | 4093209 | chr5 | 177133078 | 177300210 | STIM1_NSD1 | + | + |
| chr11 | 8106092 | 8169043 | chr7 | 142299010 | 142813287 | RIC3_TRB | − | + |
| chr11 | 8106092 | 8169043 | chr7 | 142801040 | 142802748 | RIC3_TRBC2 | − | + |
| chr11 | 20156154 | 20160613 | chr9 | 36833274 | 37034185 | DBX1_PAX5 | − | − |
| chr11 | 34051682 | 34101156 | chr5 | 150113836 | 150155860 | CAPRIN1_PDGFRB | + | − |
| chr11 | 59142747 | 59155039 | chr6 | 135181314 | 135219171 | FAM111A_MYB | + | + |
| chr11 | 62559600 | 62573973 | chr6 | 73368554 | 73395093 | EEF1G_OOEP | − | − |
| chr11 | 72002864 | 72080693 | chr17 | 40309193 | 40356796 | NUMA1_RARA | − | + |
| chr11 | 72814405 | 72843674 | chr11 | 118436463 | 118526832 | ATG16L2_KMT2A | + | + |
| chr11 | 85957687 | 86069097 | chr10 | 21524674 | 21743630 | PICALM_MLLT10 | − | + |
| chr11 | 101451563 | 101583928 | chr8 | 109240918 | 109334385 | TRPC6_NUDCD1 | − | − |
| chr11 | 114059592 | 114250676 | chr17 | 40309193 | 40356796 | ZBTB16_RARA | + | + |
| chr11 | 117427772 | 117797261 | chr11 | 118436463 | 118526832 | DSCAML1_KMT2A | − | + |
| chr11 | 117836980 | 117877486 | chr11 | 118436463 | 118526832 | FXYD6_KMT2A | − | + |
| chr11 | 118436463 | 118526832 | chr1 | 51354262 | 51519328 | KMT2A_EPS15 | + | − |
| chr11 | 118436463 | 118526832 | chr1 | 151057757 | 151068497 | KMT2A_MLLT11 | + | + |
| chr11 | 118436463 | 118526832 | chr10 | 20783905 | 21174187 | KMT2A_NEBL | + | − |
| chr11 | 118436463 | 118526832 | chr10 | 21524674 | 21743630 | KMT2A_MLLT10 | + | + |
| chr11 | 118436463 | 118526832 | chr10 | 26746595 | 26861087 | KMT2A_ABI1 | + | − |
| chr11 | 118436463 | 118526832 | chr10 | 68560655 | 68694482 | KMT2A_TET1 | + | + |
| chr11 | 118436463 | 118526832 | chr11 | 73308288 | 73369091 | KMT2A_ARHGEF17 | + | + |
| chr11 | 118436463 | 118526832 | chr11 | 85957687 | 86069097 | KMT2A_PICALM | + | − |
| chr11 | 118436463 | 118526832 | chr11 | 95976597 | 96343180 | KMT2A_MAML2 | + | − |
| chr11 | 118436463 | 118526832 | chr11 | 118436463 | 118526832 | KMT2A_KMT2A | + | + |
| chr11 | 118436463 | 118526832 | chr11 | 119206275 | 119308149 | KMT2A_CBL | + | + |
| chr11 | 118436463 | 118526832 | chr11 | 120337077 | 120489936 | KMT2A_ARHGEF12 | + | + |
| chr11 | 118436463 | 118526832 | chr12 | 55757273 | 55817756 | KMT2A_SARNP | + | − |
| chr11 | 118436463 | 118526832 | chr14 | 66507406 | 67181803 | KMT2A_GPHN | + | + |
| chr11 | 118436463 | 118526832 | chr14 | 104865279 | 104896742 | KMT2A_CEP170B | + | + |
| chr11 | 118436463 | 118526832 | chr15 | 40594019 | 40664342 | KMT2A_KNL1 | + | + |
| chr11 | 118436463 | 118526832 | chr15 | 40807088 | 40815084 | KMT2A_ZFYVE19 | + | + |
| chr11 | 118436463 | 118526832 | chr16 | 3725053 | 3880726 | KMT2A_CREBBP | + | − |
| chr11 | 118436463 | 118526832 | chr17 | 9913849 | 10198551 | KMT2A_GAS7 | + | − |
| chr11 | 118436463 | 118526832 | chr17 | 38705541 | 38729803 | KMT2A_MLLT6 | + | + |
| chr11 | 118436463 | 118526832 | chr17 | 38869858 | 38921770 | KMT2A_LASP1 | + | + |
| chr11 | 118436463 | 118526832 | chr17 | 40309193 | 40356796 | KMT2A_RARA | + | + |
| chr11 | 118436463 | 118526832 | chr19 | 4360369 | 4400547 | KMT2A_SH3GL1 | + | − |
| chr11 | 118436463 | 118526832 | chr19 | 6210378 | 6279948 | KMT2A_MLLT1 | + | − |
| chr11 | 118436463 | 118526832 | chr19 | 18442662 | 18522127 | KMT2A_ELL | + | − |
| chr11 | 118436463 | 118526832 | chr2 | 203328218 | 203447723 | KMT2A_ABI2 | + | + |
| chr11 | 118436463 | 118526832 | chr22 | 41091785 | 41180079 | KMT2A_EP300 | + | + |
| chr11 | 118436463 | 118526832 | chr3 | 48673843 | 48685927 | KMT2A_NCKIPSD | + | − |
| chr11 | 118436463 | 118526832 | chr3 | 108549868 | 108589452 | KMT2A_CIP2A | + | − |
| chr11 | 118436463 | 118526832 | chr3 | 155870535 | 155944026 | KMT2A_GMPS | + | + |
| chr11 | 118436463 | 118526832 | chr3 | 188212932 | 188890671 | KMT2A_LPP | + | + |
| chr11 | 118436463 | 118526832 | chr4 | 39822862 | 39977956 | KMT2A_PDS5A | + | − |
| chr11 | 118436463 | 118526832 | chr4 | 48497362 | 48780299 | KMT2A_FRYL | + | − |
| chr11 | 118436463 | 118526832 | chr4 | 52590971 | 52659335 | KMT2A_USP46 | + | − |
| chr11 | 118436463 | 118526832 | chr4 | 76949808 | 77040384 | KMT2A_SEPT11 | + | + |
| chr11 | 118436463 | 118526832 | chr4 | 86935001 | 87141054 | KMT2A_AFF1 | + | + |
| chr11 | 118436463 | 118526832 | chr4 | 185585443 | 185956368 | KMT2A_SORBS2 | + | − |
| chr11 | 118436463 | 118526832 | chr5 | 127517608 | 127555089 | KMT2A_PRRC1 | + | + |
| chr11 | 118436463 | 118526832 | chr5 | 139274103 | 139331671 | KMT2A_MATR3 | + | + |
| chr11 | 118436463 | 118526832 | chr5 | 142770376 | 143229011 | KMT2A_ARHGAP26 | + | + |
| chr11 | 118436463 | 118526832 | chr5 | 160251656 | 160312928 | KMT2A_CCNJL | + | − |
| chr11 | 118436463 | 118526832 | chr6 | 70667775 | 70862015 | KMT2A_SMAP1 | + | + |
| chr11 | 118436463 | 118526832 | chr6 | 89829899 | 89871412 | KMT2A_CASP8AP2 | + | + |
| chr11 | 118436463 | 118526832 | chr6 | 108559834 | 108684774 | KMT2A_FOXO3 | + | + |
| chr11 | 118436463 | 118526832 | chr6 | 136557046 | 136792518 | KMT2A_MAP3K5 | + | − |
| chr11 | 118436463 | 118526832 | chr6 | 167826990 | 167972020 | KMT2A_AFDN | + | + |
| chr11 | 118436463 | 118526832 | chr7 | 5306789 | 5423546 | KMT2A_TNRC18 | + | − |
| chr11 | 118436463 | 118526832 | chr7 | 87628412 | 87832296 | KMT2A_RUNDC3B | + | + |
| chr11 | 118436463 | 118526832 | chr9 | 20341664 | 20622543 | KMT2A_MLLT3 | + | − |
| chr11 | 118436463 | 118526832 | chr9 | 96450200 | 96491336 | KMT2A_HABP4 | + | + |
| chr11 | 118436463 | 118526832 | chr9 | 121567101 | 121785528 | KMT2A_DAB2IP | + | + |
| chr11 | 118436463 | 118526832 | chr9 | 129887186 | 130043194 | KMT2A_FNBP1 | + | − |
| chr11 | 118436463 | 118526832 | chr9 | 131009081 | 131093059 | KMT2A_LAMC3 | + | + |
| chr11 | 118436463 | 118526832 | chrX | 71096196 | 71103535 | KMT2A_FOXO4 | + | + |
| chr11 | 118436463 | 118526832 | chrX | 119615723 | 119693370 | KMT2A_SEPT6 | + | − |
| chr11 | 118436463 | 118526832 | chrX | 135760156 | 135768191 | KMT2A_CT45A3 | + | − |
| chr11 | 118436463 | 118526832 | chrX | 135811980 | 135820012 | KMT2A_CT45A2 | + | − |
| chr11 | 118436463 | 118526832 | chrX | 154348525 | 154374638 | KMT2A_FLNA | + | − |
| chr11 | 119206275 | 119308149 | chr9 | 470290 | 746105 | CBL_KANK1 | + | + |
| chr11 | 122655674 | 122814473 | chr7 | 23504779 | 23532041 | UBASH3B_TRA2A | + | − |
| chr12 | 991207 | 1495933 | chr5 | 150113836 | 150155860 | ERC1_PDGFRB | + | − |
| chr12 | 6666476 | 6689510 | chr5 | 64165881 | 64372869 | ZNF384_RNF180 | − | + |
| chr12 | 10158300 | 10172138 | chrX | 123859811 | 123913976 | OLR1_XIAP | − | + |
| chr12 | 10170541 | 10191785 | chrX | 123859811 | 123913976 | TMEM52B_XIAP | + | + |
| chr12 | 11649853 | 11895402 | chr1 | 3069210 | 3438621 | ETV6_PRDM16 | + | + |
| chr12 | 11649853 | 11895402 | chr1 | 179099326 | 179229601 | ETV6_ABL2 | + | − |
| chr12 | 11649853 | 11895402 | chr10 | 99396869 | 99431569 | ETV6_GOT1 | + | − |
| chr12 | 11649853 | 11895402 | chr12 | 11649853 | 11895402 | ETV6_ETV6 | + | + |
| chr12 | 11649853 | 11895402 | chr12 | 56595595 | 56636366 | ETV6_BAZ2A | + | − |
| chr12 | 11649853 | 11895402 | chr12 | 70638081 | 70920843 | ETV6_PTPRR | + | − |
| chr12 | 11649853 | 11895402 | chr15 | 87859750 | 88256747 | ETV6_NTRK3 | + | − |
| chr12 | 11649853 | 11895402 | chr17 | 8144054 | 8156360 | ETV6_PER1 | + | − |
| chr12 | 11649853 | 11895402 | chr18 | 44680886 | 45068510 | ETV6_SETBP1 | + | + |
| chr12 | 11649853 | 11895402 | chr3 | 41194740 | 41239949 | ETV6_CTNNB1 | + | + |
| chr12 | 11649853 | 11895402 | chr3 | 169084760 | 169663470 | ETV6_MECOM | + | − |
| chr12 | 11649853 | 11895402 | chr4 | 54009788 | 54064690 | ETV6_CHIC2 | + | − |
| chr12 | 11649853 | 11895402 | chr4 | 54229096 | 54298247 | ETV6_PDGFRA | + | + |
| chr12 | 11649853 | 11895402 | chr5 | 131949975 | 132011914 | ETV6_ACSL6 | + | − |
| chr12 | 11649853 | 11895402 | chr5 | 150113836 | 150155860 | ETV6_PDGFRB | + | − |
| chr12 | 11649853 | 11895402 | chr5 | 158695919 | 159099761 | ETV6_EBF1 | + | − |
| chr12 | 11649853 | 11895402 | chr6 | 115931148 | 116060758 | ETV6_FRK | + | − |
| chr12 | 11649853 | 11895402 | chr6 | 124962544 | 125092633 | ETV6_RNF217 | + | + |
| chr12 | 11649853 | 11895402 | chr7 | 36389805 | 36453791 | ETV6_ANLN | + | + |
| chr12 | 11649853 | 11895402 | chr7 | 36854360 | 37449249 | ETV6_ELMO1 | + | − |
| chr12 | 11649853 | 11895402 | chr8 | 55879834 | 56014168 | ETV6_LYN | + | + |
| chr12 | 11649853 | 11895402 | chr8 | 98454694 | 98942571 | ETV6_STK3 | + | − |
| chr12 | 11649853 | 11895402 | chr9 | 4985244 | 5128183 | ETV6_JAK2 | + | + |
| chr12 | 11649853 | 11895402 | chr9 | 90801786 | 90898549 | ETV6_SYK | + | + |
| chr12 | 11649853 | 11895402 | chr9 | 130713945 | 130885683 | ETV6_ABL1 | + | + |
| chr12 | 14365631 | 14502935 | chr9 | 4985244 | 5128183 | ATF7IP_JAK2 | + | + |
| chr12 | 26938382 | 26966553 | chr8 | 38411138 | 38468834 | FGFR1OP2_FGFR1 | + | − |
| chr12 | 48935722 | 48957551 | chr6 | 149317533 | 149411395 | ARF3_TAB2 | − | + |
| chr12 | 49018974 | 49055324 | chr4 | 82819010 | 82900538 | KMT2D_SEC31A | − | − |
| chr12 | 51281281 | 51324668 | chr5 | 150113836 | 150155860 | BIN2_PDGFRB | − | − |
| chr12 | 53452101 | 53481161 | chr9 | 131394092 | 131500197 | PCBP2_PRRC2B | + | + |
| chr12 | 65824130 | 65966295 | chr12 | 65824130 | 65966295 | HMGA2_HMGA2 | + | + |
| chr12 | 69239536 | 69274358 | chr8 | 38411138 | 38468834 | CPSF6_FGFR1 | + | − |
| chr12 | 71754873 | 71800285 | chr12 | 71839717 | 71927248 | RAB21_TBC1D15 | + | + |
| chr12 | 104456970 | 104762014 | chrX | 120362084 | 120383249 | CHST11_ATP1B4 | + | + |
| chr12 | 108522579 | 108561389 | chr5 | 150113836 | 150155860 | SART3_PDGFRB | − | − |
| chr12 | 109929801 | 109996389 | chr5 | 150113836 | 150155860 | GIT2_PDGFRB | − | − |
| chr12 | 117453053 | 117968983 | chr3 | 4303303 | 4317567 | KSR2_SETMAR | − | + |
| chr12 | 121429098 | 121581015 | chr5 | 132875378 | 132963634 | KDM2B_AFF4 | − | − |
| chr12 | 121429098 | 121581015 | chrY | 13248378 | 13480673 | KDM2B_UTY | − | − |
| chr12 | 124324414 | 124535603 | chr6 | 117288299 | 117425855 | NCOR2_ROS1 | − | − |
| chr12 | 132489550 | 132585188 | chr9 | 36833274 | 37034185 | FBRSL1_PAX5 | + | − |
| chr13 | 19958669 | 20091829 | chr8 | 38411138 | 38468834 | ZMYM2_FGFR1 | + | − |
| chr13 | 48303725 | 48481890 | chr7 | 15200317 | 15562015 | RB1_AGMO | + | − |
| chr14 | 21621903 | 22552132 | chr14 | 95709966 | 95714196 | TRA_TCL1A | + | − |
| chr14 | 21621903 | 22552132 | chr6 | 118460780 | 118710075 | TRA_CEP85L | + | − |
| chr14 | 21621903 | 22552132 | chr7 | 142299010 | 142813287 | TRA_TRB | + | + |
| chr14 | 21621903 | 22552132 | chr9 | 21968104 | 21995301 | TRA_CDKN2A | + | − |
| chr14 | 21621903 | 22552132 | chr9 | 136494432 | 136545786 | TRA_NOTCH1 | + | − |
| chr14 | 21621903 | 22552132 | chrX | 155065320 | 155147775 | TRA_MTCP1 | + | − |
| chr14 | 22422545 | 22466577 | chr14 | 95709966 | 95714196 | TRD_TCL1A | + | − |
| chr14 | 22422545 | 22466577 | chr5 | 170861869 | 171300015 | TRD_RANBP17 | + | + |
| chr14 | 22422545 | 22466577 | chr5 | 171309283 | 171312134 | TRD_TLX3 | + | + |
| chr14 | 22422545 | 22466577 | chr5 | 173232108 | 173235357 | TRD_NKX2-5 | + | − |
| chr14 | 22422545 | 22466577 | chr8 | 127736068 | 127741434 | TRD_MYC | + | + |
| chr14 | 22422545 | 22466577 | chr8 | 127890627 | 128101253 | TRD_PVT1 | + | + |
| chr14 | 22422545 | 22466577 | chr9 | 77716086 | 78031458 | TRD_GNAQ | + | − |
| chr14 | 30893798 | 31026401 | chr9 | 4985244 | 5128183 | STRN3_JAK2 | − | + |
| chr14 | 50719762 | 50831121 | chr5 | 150113836 | 150155860 | NIN_PDGFRB | − | − |
| chr14 | 73958009 | 74015928 | chr6 | 135283531 | 135497745 | ENTPD5_AHI1 | − | − |
| chr14 | 91271324 | 91417777 | chr5 | 150113836 | 150155860 | CCDC88C_PDGFRB | − | − |
| chr14 | 91965990 | 92040059 | chr5 | 150113836 | 150155860 | TRIP11_PDGFRB | − | − |
| chr14 | 99169286 | 99271485 | chr14 | 99169286 | 99271485 | BCL11B_BCL11B | − | − |
| chr14 | 99169286 | 99271485 | chr5 | 173232108 | 173235357 | BCL11B_NKX2-5 | − | − |
| chr14 | 105586436 | 106879844 | chr3 | 187721376 | 187745727 | IGH_BCL6 | − | − |
| chr14 | 105586436 | 106879844 | chr4 | 190173773 | 190175845 | IGH_DUX4 | − | + |
| chr14 | 105586436 | 106879844 | chr5 | 1253166 | 1295047 | IGH_TERT | − | − |
| chr14 | 105586436 | 106879844 | chr5 | 112976735 | 113020970 | IGH_DCP2 | − | + |
| chr14 | 105586436 | 106879844 | chr5 | 158695919 | 159099761 | IGH_EBF1 | − | − |
| chr14 | 105586436 | 106879844 | chr6 | 391738 | 411447 | IGH_IRF4 | − | + |
| chr14 | 105586436 | 106879844 | chr7 | 55019100 | 55211628 | IGH_EGFR | − | + |
| chr14 | 105586436 | 106879844 | chr7 | 92468379 | 92477915 | IGH_ERVW-1 | − | − |
| chr14 | 105586436 | 106879844 | chr7 | 110663053 | 111562517 | IGH_IMMP2L | − | − |
| chr14 | 105586436 | 106879844 | chr8 | 47736908 | 47739086 | IGH_CEBPD | − | − |
| chr14 | 105586436 | 106879844 | chr9 | 36833274 | 37034185 | IGH_PAX5 | − | − |
| chr14 | 105586436 | 106879844 | chr9 | 124011609 | 124033301 | IGH_LHX2 | − | + |
| chr15 | 43407208 | 43510614 | chr5 | 150113836 | 150155860 | TP53BP1_PDGFRB | − | − |
| chr15 | 43510957 | 43531620 | chr9 | 128947698 | 129007096 | MAP1A_NUP188 | + | + |
| chr15 | 73994728 | 74047812 | chr17 | 40309193 | 40356796 | PML_RARA | + | + |
| chr15 | 75370932 | 75455783 | chr9 | 33524393 | 33573001 | SIN3A_ANKRD18B | − | + |
| chr15 | 80947328 | 80989828 | chrX | 41514933 | 41923169 | MESD_CASK | − | − |
| chr15 | 91853855 | 92172435 | chr7 | 36854360 | 37449249 | SLCO3A1_ELMO1 | + | − |
| chr16 | 176679 | 177522 | chr5 | 150401669 | 150412929 | HBA1_CD74 | + | − |
| chr16 | 3725053 | 3880726 | chr7 | 142645960 | 142646467 | CREBBP_TRBV23-1 | − | + |
| chr16 | 3725053 | 3880726 | chr8 | 41929478 | 42052026 | CREBBP_KAT6A | − | − |
| chr16 | 10877197 | 10936388 | chr9 | 5450502 | 5470566 | CIITA_CD274 | + | + |
| chr16 | 10877197 | 10936388 | chr9 | 5510569 | 5571254 | CIITA_PDCD1LG2 | + | + |
| chr16 | 10877197 | 10936388 | chr9 | 133097719 | 133149334 | CIITA_RALGDS | + | − |
| chr16 | 11976737 | 12574289 | chr9 | 4985244 | 5128183 | SNX29_JAK2 | + | + |
| chr16 | 15643266 | 15726353 | chr5 | 150113836 | 150155860 | NDE1_PDGFRB | + | − |
| chr16 | 31180109 | 31194871 | chr21 | 38380029 | 38661780 | FUS_ERG | + | − |
| chr16 | 31180109 | 31194871 | chr9 | 128683654 | 128696400 | FUS_SET | + | + |
| chr16 | 67029146 | 67101058 | chr16 | 15703171 | 15857011 | CBFB_MYH11 | + | − |
| chr16 | 88874857 | 88977204 | chr16 | 4314760 | 4339597 | CBFA2T3_GLIS2 | − | + |
| chr17 | 5282299 | 5385812 | chr5 | 150113836 | 150155860 | RABEP1_PDGFRB | + | − |
| chr17 | 16029156 | 16215549 | chr8 | 55879834 | 56014168 | NCOR1_LYN | − | + |
| chr17 | 17042759 | 17192648 | chr5 | 150113836 | 150155860 | MPRIP_PDGFRB | + | − |
| chr17 | 20009362 | 20314138 | chr5 | 150113836 | 150155860 | SPECC1_PDGFRB | + | − |
| chr17 | 27456469 | 27626435 | chrX | 124375902 | 124963817 | KSR1_TENM1 | + | − |
| chr17 | 29071123 | 29180412 | chr5 | 150113836 | 150155860 | MYO18A_PDGFRB | − | − |
| chr17 | 29071123 | 29180412 | chr8 | 38411138 | 38468834 | MYO18A_FGFR1 | − | − |
| chr17 | 42199167 | 42276406 | chr17 | 40309193 | 40356796 | STAT5B_RARA | − | + |
| chr17 | 50183288 | 50201632 | chr5 | 150113836 | 150155860 | COL1A1_PDGFRB | − | − |
| chr17 | 50962173 | 51120865 | chr9 | 4985244 | 5128183 | SPAG9_JAK2 | − | + |
| chr17 | 57256533 | 57684685 | chr17 | 57256533 | 57684685 | MSI2_MSI2 | + | + |
| chr17 | 57256533 | 57684685 | chr7 | 27162434 | 27165530 | MSI2_HOXA9 | + | − |
| chr17 | 68515399 | 68551319 | chr17 | 40309193 | 40356796 | PRKAR1A_RARA | + | + |
| chr17 | 80260867 | 80398786 | chr8 | 127736068 | 127741434 | RNF213_MYC | + | + |
| chr17 | 82519714 | 82604607 | chr7 | 74289474 | 74405943 | FOXK2_CLIP2 | + | + |
| chr18 | 2847029 | 2915993 | chr9 | 36833274 | 37034185 | EMILIN2_PAX5 | + | − |
| chr18 | 12785477 | 12884338 | chr5 | 159263080 | 159286040 | PTPN2_UBLCP1 | − | + |
| chr18 | 58671385 | 58754477 | chr3 | 47850695 | 48088839 | MALT1_MAP4 | + | − |
| chr19 | 2360237 | 2426239 | chr9 | 36833274 | 37034185 | TMPRSS9_PAX5 | + | − |
| chr19 | 10718092 | 10831884 | chr7 | 93188585 | 93226524 | DNM2_HEPACAM2 | + | − |
| chr19 | 13099032 | 13102867 | chr7 | 142299010 | 142813287 | LYL1_TRB | − | + |
| chr19 | 19385832 | 19508931 | chr8 | 55879834 | 56014168 | GATAD2A_LYN | + | + |
| chr19 | 21020619 | 21060050 | chr9 | 4985244 | 5128183 | ZNF430_JAK2 | + | + |
| chr19 | 34172565 | 34229515 | chr9 | 130713945 | 130885683 | LSM14A_ABL1 | + | + |
| chr19 | 44748546 | 44760044 | chr8 | 127736068 | 127741434 | BCL3_MYC | + | + |
| chr19 | 58183028 | 58213562 | chr9 | 4985244 | 5128183 | ZNF274_JAK2 | + | + |
| chr19 | 58305318 | 58315663 | chr8 | 38411138 | 38468834 | ERVK3-1_FGFR1 | + | − |
| chr2 | 32946971 | 33399359 | chr2 | 32357027 | 32618899 | LTBP1_BIRC6 | + | + |
| chr2 | 43230835 | 43596046 | chr3 | 169084760 | 169663470 | THADA_MECOM | − | − |
| chr2 | 54456316 | 54671445 | chr5 | 150113836 | 150155860 | SPTBN1_PDGFRB | + | − |
| chr2 | 60450519 | 60554467 | chr3 | 169084760 | 169663470 | BCL11A_MECOM | − | − |
| chr2 | 108719480 | 108785811 | chr2 | 29192773 | 29921566 | RANBP2_ALK | + | − |
| chr2 | 108719480 | 108785811 | chr8 | 38411138 | 38468834 | RANBP2_FGFR1 | + | − |
| chr2 | 108719480 | 108785811 | chr9 | 130713945 | 130885683 | RANBP2_ABL1 | + | + |
| chr2 | 191678135 | 191696659 | chr17 | 40309193 | 40356796 | NABP1_RARA | + | + |
| chr2 | 237627575 | 237780315 | chr8 | 38411138 | 38468834 | LRRFIP1_FGFR1 | + | − |
| chr20 | 18587892 | 18763917 | chr5 | 150113836 | 150155860 | DTD1_PDGFRB | + | − |
| chr20 | 32277663 | 32335011 | chr7 | 142299010 | 142813287 | KIF3B_TRB | + | + |
| chr20 | 34714773 | 34825649 | chr8 | 70051650 | 70071327 | NCOA6_PRDM14 | − | − |
| chr20 | 44496223 | 44522085 | chr9 | 37120538 | 37358149 | SERINC3_ZCCHC7 | − | + |
| chr20 | 47209213 | 47356889 | chr5 | 150113836 | 150155860 | ZMYND8_PDGFRB | − | − |
| chr20 | 52051662 | 52191779 | chr21 | 34787800 | 35049344 | ZFP64_RUNX1 | − | − |
| chr21 | 14961234 | 15065000 | chr3 | 169084760 | 169663470 | NRIP1_MECOM | − | − |
| chr21 | 15730024 | 15880069 | chr9 | 4985244 | 5128183 | USP25_JAK2 | + | + |
| chr21 | 29024628 | 29054488 | chr21 | 34787800 | 35049344 | USP16_RUNX1 | + | − |
| chr21 | 34516483 | 34615142 | chr4 | 123396794 | 123403760 | RCAN1_SPRY1 | − | + |
| chr21 | 34787800 | 35049344 | chr1 | 3069210 | 3438621 | RUNX1_PRDM16 | − | + |
| chr21 | 34787800 | 35049344 | chr1 | 28736620 | 28769774 | RUNX1_YTHDF2 | − | + |
| chr21 | 34787800 | 35049344 | chr1 | 86424085 | 86456558 | RUNX1_CLCA2 | − | + |
| chr21 | 34787800 | 35049344 | chr1 | 151282311 | 151291903 | RUNX1_ZNF687 | − | + |
| chr21 | 34787800 | 35049344 | chr11 | 33542274 | 33674102 | RUNX1_KIAA1549L | − | + |
| chr21 | 34787800 | 35049344 | chr11 | 58526870 | 58578166 | RUNX1_LPXN | − | − |
| chr21 | 34787800 | 35049344 | chr11 | 63998557 | 64166061 | RUNX1_MACROD1 | − | − |
| chr21 | 34787800 | 35049344 | chr16 | 88874857 | 88977204 | RUNX1_CBFA2T3 | − | − |
| chr21 | 34787800 | 35049344 | chr21 | 34787800 | 35049344 | RUNX1_RUNX1 | − | − |
| chr21 | 34787800 | 35049344 | chr3 | 169084760 | 169663470 | RUNX1_MECOM | − | − |
| chr21 | 34787800 | 35049344 | chr3 | 169483670 | 169484080 | RUNX1_RPL22P1 | − | − |
| chr21 | 34787800 | 35049344 | chr4 | 151120286 | 151325632 | RUNX1_SH3D19 | − | − |
| chr21 | 34787800 | 35049344 | chr5 | 127517608 | 127555089 | RUNX1_PRRC1 | − | + |
| chr21 | 34787800 | 35049344 | chr5 | 129460264 | 129738683 | RUNX1_ADAMTS19 | − | + |
| chr21 | 34787800 | 35049344 | chr6 | 17582033 | 17582305 | RUNX1_SUMO2P13 | − | + |
| chr21 | 34787800 | 35049344 | chr7 | 6104883 | 6161564 | RUNX1_USP42 | − | + |
| chr21 | 34787800 | 35049344 | chr7 | 27242699 | 27247825 | RUNX1_EVX1 | − | + |
| chr21 | 34787800 | 35049344 | chr8 | 91954966 | 92103226 | RUNX1_RUNX1T1 | − | − |
| chr21 | 34787800 | 35049344 | chr8 | 105318691 | 105804532 | RUNX1_ZFPM2 | − | + |
| chr21 | 34787800 | 35049344 | chr8 | 115408495 | 115669001 | RUNX1_TRPS1 | − | − |
| chr21 | 34787800 | 35049344 | chrX | 23667445 | 23686399 | RUNX1_PRDX4 | − | + |
| chr21 | 38380029 | 38661780 | chr4 | 190173773 | 190175845 | ERG_DUX4 | − | + |
| chr21 | 42653749 | 42775509 | chr8 | 85656441 | 85663039 | PDE9A_REXO1L1P | + | − |
| chr22 | 22026075 | 22922913 | chr4 | 1793292 | 1808872 | IGL_FGFR3 | + | + |
| chr22 | 22026075 | 22922913 | chr6 | 391738 | 411447 | IGL_IRF4 | + | + |
| chr22 | 22026075 | 22922913 | chr6 | 41934933 | 42050357 | IGL_CCND3 | + | − |
| chr22 | 22026075 | 22922913 | chr8 | 127890627 | 128101253 | IGL_PVT1 | + | + |
| chr22 | 23180209 | 23318037 | chr4 | 54229096 | 54298247 | BCR_PDGFRA | + | + |
| chr22 | 23180209 | 23318037 | chr8 | 38411138 | 38468834 | BCR_FGFR1 | + | − |
| chr22 | 23180209 | 23318037 | chr9 | 4985244 | 5128183 | BCR_JAK2 | + | + |
| chr22 | 23180209 | 23318037 | chr9 | 126914773 | 127223164 | BCR_RALGPS1 | + | + |
| chr22 | 23180209 | 23318037 | chr9 | 130713945 | 130885683 | BCR_ABL1 | + | + |
| chr22 | 41091785 | 41180079 | chr7 | 27153715 | 27156677 | EP300_HOXA7 | + | − |
| chr3 | 9397718 | 9478154 | chr7 | 50304668 | 50405101 | SETD5_IKZF1 | + | + |
| chr3 | 10115591 | 10127190 | chr3 | 10141007 | 10152220 | BRK1_VHL | + | + |
| chr3 | 12583600 | 12664226 | chr3 | 12734706 | 12769457 | RAF1_TMEM40 | − | − |
| chr3 | 15560703 | 15601852 | chr3 | 15450132 | 15521751 | HACL1_COLQ | − | − |
| chr3 | 15667235 | 15859771 | chr11 | 3675009 | 3797792 | ANKRD28_NUP98 | − | − |
| chr3 | 16315855 | 16513706 | chr8 | 127890627 | 128101253 | RFTN1_PVT1 | − | + |
| chr3 | 28241594 | 28325142 | chr3 | 27372720 | 27484420 | CMC1_SLC4A7 | + | − |
| chr3 | 30606501 | 30694134 | chr19 | 4044363 | 4066945 | TGFBR2_ZBTB7A | + | − |
| chr3 | 37243176 | 37366751 | chr5 | 150113836 | 150155860 | GOLGA4_PDGFRB | + | − |
| chr3 | 37988058 | 38007188 | chr9 | 137007233 | 137028922 | VILL_ABCA2 | + | − |
| chr3 | 39051997 | 39096388 | chr5 | 150113836 | 150155860 | WDR48_PDGFRB | + | − |
| chr3 | 47016688 | 47163967 | chr3 | 46921730 | 46982010 | SETD2_CCDC12 | − | − |
| chr3 | 47585271 | 47781917 | chr17 | 47970533 | 47981772 | SMARCC1_CDK5RAP3 | − | + |
| chr3 | 47850695 | 48088839 | chr18 | 58671385 | 58754477 | MAP4_MALT1 | − | + |
| chr3 | 48599002 | 48610037 | chr3 | 43365847 | 43622068 | UQCRC1_ANO10 | − | − |
| chr3 | 48673843 | 48685927 | chr3 | 48636468 | 48662915 | NCKIPSD_CELSR3 | − | − |
| chr3 | 49940006 | 50077249 | chr5 | 150053290 | 150113372 | RBM6_CSF1R | + | − |
| chr3 | 100609588 | 100695479 | chr3 | 100709360 | 100748942 | ADGRG7_TFG | + | + |
| chr3 | 100709360 | 100748942 | chr3 | 100609588 | 100695479 | TFG_ADGRG7 | + | + |
| chr3 | 101324204 | 101513184 | chr12 | 11649853 | 11895402 | SENP7_ETV6 | − | + |
| chr3 | 121663202 | 121749767 | chr13 | 28003273 | 28100592 | GOLGB1_FLT3 | − | − |
| chr3 | 121663202 | 121749767 | chr5 | 150113836 | 150155860 | GOLGB1_PDGFRB | − | − |
| chr3 | 128479426 | 128493185 | chr3 | 169084760 | 169663470 | GATA2_MECOM | − | − |
| chr3 | 128479426 | 128493185 | chr7 | 27162434 | 27165530 | GATA2_HOXA9 | − | − |
| chr3 | 128479426 | 128493185 | chr7 | 27171219 | 27180261 | GATA2_HOXA10 | − | − |
| chr3 | 128571999 | 128576086 | chr3 | 169084760 | 169663470 | LINC01565_MECOM | − | − |
| chr3 | 128620158 | 128681075 | chr1 | 3069210 | 3438621 | RPN1_PRDM16 | − | + |
| chr3 | 128620158 | 128681075 | chr3 | 169084760 | 169663470 | RPN1_MECOM | − | − |
| chr3 | 134157132 | 134250744 | chr21 | 33903452 | 33915980 | RYK_ATP5PO | − | − |
| chr3 | 136148921 | 136195846 | chr3 | 177019354 | 177196478 | MSL2_TBL1XR1 | − | − |
| chr3 | 152268039 | 152465779 | chr9 | 36833274 | 37034185 | MBNL1_PAX5 | + | − |
| chr3 | 152268039 | 152465779 | chr9 | 130713945 | 130885683 | MBNL1_ABL1 | + | + |
| chr3 | 160494994 | 160565588 | chr9 | 109640787 | 109946703 | KPNA4_PALM2 | − | + |
| chr3 | 169084760 | 169663470 | chr21 | 34787800 | 35049344 | MECOM_RUNX1 | − | − |
| chr3 | 169084760 | 169663470 | chr3 | 169084760 | 169663470 | MECOM_MECOM | − | − |
| chr3 | 169084760 | 169663470 | chr7 | 92604920 | 92833917 | MECOM_CDK6 | − | − |
| chr3 | 169084760 | 169663470 | chr7 | 142299010 | 142813287 | MECOM_TRB | − | + |
| chr3 | 172040553 | 172401665 | chr3 | 169084760 | 169663470 | FNDC3B_MECOM | + | − |
| chr3 | 177019354 | 177196478 | chr17 | 40309193 | 40356796 | TBL1XR1_RARA | − | + |
| chr3 | 177019354 | 177196478 | chr3 | 139357405 | 139389732 | TBL1XR1_COPB2 | − | − |
| chr3 | 177019354 | 177196478 | chr3 | 189631426 | 189897279 | TBL1XR1_TP63 | − | + |
| chr3 | 177019354 | 177196478 | chr5 | 150053290 | 150113372 | TBL1XR1_CSF1R | − | − |
| chr3 | 180912301 | 180982753 | chr3 | 177019354 | 177196478 | FXR1_TBL1XR1 | + | − |
| chr3 | 186147200 | 186362237 | chr4 | 15703095 | 15732787 | DGKG_BST1 | − | + |
| chr3 | 187721376 | 187745727 | chr13 | 46125919 | 46211348 | BCL6_LCP1 | − | − |
| chr3 | 187721376 | 187745727 | chr16 | 10877197 | 10936388 | BCL6_CIITA | − | + |
| chr3 | 187721376 | 187745727 | chr19 | 6677703 | 6720682 | BCL6_C3 | − | − |
| chr3 | 187721376 | 187745727 | chr3 | 152268039 | 152465779 | BCL6_MBNL1 | − | + |
| chr3 | 187721376 | 187745727 | chr6 | 37170202 | 37175426 | BCL6_PIM1 | − | + |
| chr3 | 187721376 | 187745727 | chr8 | 66562174 | 66613249 | BCL6_MYBL1 | − | − |
| chr3 | 188212932 | 188890671 | chr3 | 187721376 | 187745727 | LPP_BCL6 | + | − |
| chr3 | 189631426 | 189897279 | chr3 | 177019354 | 177196478 | TP63_TBL1XR1 | + | − |
| chr4 | 1009978 | 1026891 | chr4 | 1211447 | 1249137 | FGFRL1_CTBP1 | + | − |
| chr4 | 1289850 | 1340137 | chr4 | 1211447 | 1249137 | MAEA_CTBP1 | + | − |
| chr4 | 1347315 | 1395989 | chr4 | 1289850 | 1340137 | UVSSA_MAEA | + | + |
| chr4 | 26860690 | 27025381 | chr7 | 50304668 | 50405101 | STIM2_IKZF1 | + | + |
| chr4 | 53377644 | 53459668 | chr17 | 40309193 | 40356796 | FIP1L1_RARA | + | + |
| chr4 | 53377644 | 53459668 | chr4 | 54229096 | 54298247 | FIP1L1_PDGFRA | + | + |
| chr4 | 54009788 | 54064690 | chr12 | 11649853 | 11895402 | CHIC2_ETV6 | − | + |
| chr4 | 54229096 | 54298247 | chr10 | 91798311 | 91865276 | PDGFRA_TNKS2 | + | + |
| chr4 | 54229096 | 54298247 | chr12 | 11649853 | 11895402 | PDGFRA_ETV6 | + | + |
| chr4 | 67468747 | 67545606 | chr9 | 130713945 | 130885683 | CENPC_ABL1 | − | + |
| chr4 | 78776377 | 78912185 | chr12 | 6666476 | 6689510 | BMP2K_ZNF384 | + | − |
| chr4 | 81087369 | 81215117 | chr5 | 150113836 | 150155860 | PRKG2_PDGFRB | − | − |
| chr4 | 86935001 | 87141054 | chr11 | 117427772 | 117797261 | AFF1_DSCAML1 | + | − |
| chr4 | 86935001 | 87141054 | chr11 | 117836980 | 117877486 | AFF1_FXYD6 | + | − |
| chr4 | 86935001 | 87141054 | chr11 | 118998141 | 119015745 | AFF1_CCDC84 | + | + |
| chr4 | 86935001 | 87141054 | chr14 | 67819828 | 68730218 | AFF1_RAD51B | + | + |
| chr4 | 88592422 | 88708542 | chr4 | 88709788 | 88730103 | HERC3_FAM13A-AS1 | + | + |
| chr4 | 108047544 | 108168956 | chr6 | 42050521 | 42087461 | LEF1_TAF8 | − | + |
| chr4 | 139716752 | 140154184 | chr4 | 77157150 | 77170059 | MAML3_CCNG2 | − | + |
| chr4 | 150264658 | 151015727 | chr4 | 151120286 | 151325632 | LRBA_SH3D19 | − | − |
| chr4 | 158124473 | 158173050 | chr4 | 158210248 | 158255411 | FAM198B_TMEM144 | − | + |
| chr4 | 190173773 | 190175845 | chr10 | 133623894 | 133626792 | DUX4_FRG2B | + | − |
| chr4 | 190173773 | 190175845 | chr14 | 105586436 | 106879844 | DUX4_IGH | + | − |
| chr4 | 190173773 | 190175845 | chr21 | 38380029 | 38661780 | DUX4_ERG | + | − |
| chr4 | 190173773 | 190175845 | chr4 | 118467589 | 118554100 | DUX4_CEP170P1 | + | + |
| chr5 | 864272 | 892824 | chr15 | 34343314 | 34357737 | BRD9_NUTM1 | − | + |
| chr5 | 35856848 | 35879603 | chr7 | 142801040 | 142802748 | IL7R_TRBC2 | + | + |
| chr5 | 36876758 | 37066413 | chr12 | 11649853 | 11895402 | NIPBL_ETV6 | + | + |
| chr5 | 36876758 | 37066413 | chr17 | 48621158 | 48626356 | NIPBL_HOXB9 | + | − |
| chr5 | 36876758 | 37066413 | chr7 | 27162434 | 27165530 | NIPBL_HOXA9 | + | − |
| chr5 | 40759378 | 40798198 | chr5 | 40512332 | 40755908 | PRKAA1_TTC33 | − | − |
| chr5 | 55307759 | 55425581 | chr11 | 116843401 | 117098421 | MTREX_SIK3 | + | − |
| chr5 | 56815573 | 56896152 | chr16 | 58157906 | 58197920 | MAP3K1_CSNK2A2 | + | − |
| chr5 | 64165881 | 64372869 | chr15 | 85380714 | 85749355 | RNF180_AKAP13 | + | + |
| chr5 | 65517765 | 65563171 | chr11 | 118436463 | 118526832 | CENPK_KMT2A | − | + |
| chr5 | 81413020 | 81751253 | chr5 | 108747821 | 109196841 | SSBP2_FER | − | + |
| chr5 | 81413020 | 81751253 | chr5 | 150053290 | 150113372 | SSBP2_CSF1R | − | − |
| chr5 | 83940553 | 84384793 | chr7 | 131110095 | 131487844 | EDIL3_MKLN1 | − | + |
| chr5 | 88718240 | 88904257 | chr11 | 118436463 | 118526832 | MEF2C_KMT2A | − | + |
| chr5 | 98853984 | 98928957 | chr21 | 34787800 | 35049344 | CHD1_RUNX1 | − | − |
| chr5 | 112976735 | 113020970 | chr14 | 61695539 | 61748258 | DCP2_HIF1A | + | + |
| chr5 | 122774995 | 122830108 | chr9 | 130713945 | 130885683 | SNX2_ABL1 | + | + |
| chr5 | 132060528 | 132063204 | chr14 | 105586436 | 106879844 | IL3_IGH | + | − |
| chr5 | 134148934 | 134177038 | chr5 | 132875378 | 132963634 | SKP1_AFF4 | − | − |
| chr5 | 140691425 | 140699318 | chr5 | 140700333 | 140706676 | HARS2_ZMAT2 | + | + |
| chr5 | 141639301 | 141651419 | chr7 | 140719326 | 140924810 | FCHSD1_BRAF | − | − |
| chr5 | 144158158 | 144170659 | chr5 | 143277930 | 143435512 | YIPF5_NR3C1 | − | − |
| chr5 | 150113836 | 150155860 | chr14 | 91271324 | 91417777 | PDGFRB_CCDC88C | − | − |
| chr5 | 150113836 | 150155860 | chr14 | 91965990 | 92040059 | PDGFRB_TRIP11 | − | − |
| chr5 | 150113836 | 150155860 | chr17 | 29071123 | 29180412 | PDGFRB_MYO18A | − | − |
| chr5 | 150113836 | 150155860 | chr20 | 18587892 | 18763917 | PDGFRB_DTD1 | − | + |
| chr5 | 150401669 | 150412929 | chr5 | 150113836 | 150155860 | CD74_PDGFRB | − | − |
| chr5 | 151029948 | 151087158 | chr5 | 150113836 | 150155860 | TNIP1_PDGFRB | − | − |
| chr5 | 157785742 | 157859145 | chr5 | 88718240 | 88904257 | CLINT1_MEF2C | − | − |
| chr5 | 158695919 | 159099761 | chr9 | 4985244 | 5128183 | EBF1_JAK2 | − | + |
| chr5 | 170861869 | 171300015 | chr14 | 22422545 | 22466577 | RANBP17_TRD | + | + |
| chr5 | 171309283 | 171312134 | chr14 | 99169286 | 99271485 | TLX3_BCL11B | + | − |
| chr5 | 171387115 | 171410883 | chr17 | 40309193 | 40356796 | NPM1_RARA | + | + |
| chr5 | 171387115 | 171410883 | chr19 | 10350532 | 10380676 | NPM1_TYK2 | + | − |
| chr5 | 171387115 | 171410883 | chr3 | 158571162 | 158606460 | NPM1_MLF1 | + | + |
| chr5 | 172983756 | 173035445 | chr12 | 62260337 | 62416389 | ATP6V0E1_USP15 | + | + |
| chr5 | 173056351 | 173139284 | chr14 | 96392110 | 96489427 | CREBRF_AK7 | + | + |
| chr5 | 177133078 | 177300210 | chr11 | 3675009 | 3797792 | NSD1_NUP98 | + | − |
| chr5 | 177133078 | 177300210 | chr11 | 61792636 | 61797244 | NSD1_FEN1 | + | + |
| chr5 | 177331561 | 177351852 | chr14 | 96502372 | 96567111 | LMAN2_PAPOLA | − | + |
| chr5 | 177331561 | 177351852 | chr5 | 177133078 | 177300210 | LMAN2_NSD1 | − | + |
| chr5 | 179614178 | 179624669 | chr10 | 21524674 | 21743630 | HNRNPH1_MLLT10 | − | + |
| chr5 | 179614178 | 179624669 | chr21 | 38380029 | 38661780 | HNRNPH1_ERG | − | − |
| chr5 | 179820758 | 179838078 | chr8 | 38411138 | 38468834 | SQSTM1_FGFR1 | + | − |
| chr5 | 179820758 | 179838078 | chr9 | 131125560 | 131234670 | SQSTM1_NUP214 | + | + |
| chr6 | 5998001 | 6007605 | chr19 | 2511218 | 2702709 | NRN1_GNG7 | − | − |
| chr6 | 17615034 | 17706834 | chr9 | 130713945 | 130885683 | NUP153_ABL1 | − | + |
| chr6 | 18223867 | 18264823 | chr9 | 131125560 | 131234670 | DEK_NUP214 | − | + |
| chr6 | 31676739 | 31680377 | chr6 | 31686948 | 31703444 | LY6G5C_ABHD16A | − | − |
| chr6 | 33620364 | 33696574 | chr6 | 36243202 | 36308595 | ITPR3_PNPLA1 | + | + |
| chr6 | 39299000 | 39314553 | chr6 | 39329989 | 39725405 | KCNK17_KIF6 | − | − |
| chr6 | 41934933 | 42050357 | chr12 | 11649853 | 11895402 | CCND3_ETV6 | − | + |
| chr6 | 44219504 | 44234151 | chr6 | 44246165 | 44253888 | SLC29A1_HSP90AB1 | + | + |
| chr6 | 45898450 | 46080348 | chr6 | 44828731 | 45377933 | CLIC5_SUPT3H | − | − |
| chr6 | 69675950 | 69867236 | chr20 | 41402100 | 41618494 | LMBRD1_CHD6 | − | − |
| chr6 | 75602046 | 75718278 | chr6 | 123804140 | 124825657 | SENP6_NKAIN2 | + | + |
| chr6 | 85660949 | 85678748 | chr6 | 85505495 | 85593913 | SNHG5_SNX14 | − | − |
| chr6 | 89926528 | 90296908 | chr11 | 19117128 | 19176415 | BACH2_ZDHHC13 | − | + |
| chr6 | 106969830 | 106975465 | chrX | 48574523 | 48579066 | CD24_RBM3 | − | + |
| chr6 | 115931148 | 116060758 | chr12 | 11649853 | 11895402 | FRK_ETV6 | − | + |
| chr6 | 124962544 | 125092633 | chr12 | 11649853 | 11895402 | RNF217_ETV6 | + | + |
| chr6 | 130013698 | 130141449 | chr6 | 127968778 | 128520674 | L3MBTL3_PTPRK | + | − |
| chr6 | 135181314 | 135219171 | chr16 | 89644430 | 89657845 | MYB_CHMP1A | + | − |
| chr6 | 135181314 | 135219171 | chr5 | 71455614 | 71567820 | MYB_BDP1 | + | + |
| chr6 | 135181314 | 135219171 | chr6 | 135283531 | 135497745 | MYB_AHI1 | + | − |
| chr6 | 135181314 | 135219171 | chr6 | 143940301 | 144064599 | MYB_PLAGL1 | + | − |
| chr6 | 135181314 | 135219171 | chr7 | 156994050 | 157009075 | MYB_MNX1 | + | − |
| chr6 | 135181314 | 135219171 | chrX | 48786553 | 48794308 | MYB_GATA1 | + | + |
| chr6 | 138773508 | 138793317 | chr11 | 3675009 | 3797792 | CCDC28A_NUP98 | + | − |
| chr6 | 143060853 | 143340290 | chr17 | 30477361 | 30527592 | AIG1_GOSR1 | + | + |
| chr6 | 144291828 | 144853034 | chr14 | 73958009 | 74015928 | UTRN_ENTPD5 | + | − |
| chr6 | 156776019 | 157210779 | chr12 | 6666476 | 6689510 | ARID1B_ZNF384 | + | − |
| chr6 | 166999181 | 167052713 | chr10 | 43077026 | 43130351 | FGFR1OP_RET | + | + |
| chr6 | 166999181 | 167052713 | chr8 | 38411138 | 38468834 | FGFR1OP_FGFR1 | + | − |
| chr6 | 167826990 | 167972020 | chr11 | 118436463 | 118526832 | AFDN_KMT2A | + | + |
| chr7 | 876551 | 896434 | chr7 | 5306789 | 5423546 | GET4_TNRC18 | + | − |
| chr7 | 2631950 | 2664802 | chr7 | 1815793 | 2233243 | TTYH3_MAD1L1 | + | − |
| chr7 | 5190187 | 5233826 | chr12 | 112013315 | 112023451 | WIPI2_ERP29 | + | + |
| chr7 | 6374522 | 6403977 | chr6 | 75084325 | 75206051 | RAC1_COL12A1 | + | − |
| chr7 | 10931950 | 10940256 | chr7 | 12570685 | 12660179 | NDUFA4_SCIN | − | + |
| chr7 | 16646130 | 16706523 | chr7 | 65960683 | 65982314 | BZW2_GUSB | + | − |
| chr7 | 27162434 | 27165530 | chr8 | 107251511 | 107336522 | HOXA9_ANGPT1 | − | − |
| chr7 | 27168898 | 27171915 | chr6 | 109366513 | 109382812 | HOXA10-AS_CD164 | + | − |
| chr7 | 27171219 | 27180261 | chr19 | 1086578 | 1095357 | HOXA10_POLR2E | − | − |
| chr7 | 27171219 | 27180261 | chr7 | 142299010 | 142813287 | HOXA10_TRB | − | + |
| chr7 | 27181514 | 27185216 | chr7 | 16646130 | 16706523 | HOXA11_BZW2 | − | + |
| chr7 | 27184670 | 27189169 | chr7 | 92604920 | 92833917 | HOXA11-AS_CDK6 | + | − |
| chr7 | 27201843 | 27207259 | chr3 | 18347939 | 18438773 | HOTTIP_SATB1 | + | − |
| chr7 | 30289793 | 30289890 | chr7 | 30284306 | 30367692 | MIR550A1_ZNRF2 | + | + |
| chr7 | 34928875 | 35038041 | chr7 | 27181514 | 27185216 | DPY19L1_HOXA11 | − | − |
| chr7 | 37683871 | 37741374 | chr15 | 52756988 | 52790012 | GPR141_ONECUT1 | + | − |
| chr7 | 38240023 | 38368055 | chr14 | 95709966 | 95714196 | TRG_TCL1A | − | − |
| chr7 | 40126022 | 40134652 | chr6 | 27866848 | 27867529 | MPLKIP_HIST1H1B | − | − |
| chr7 | 43112598 | 43566001 | chr5 | 150113836 | 150155860 | HECW1_PDGFRB | + | − |
| chr7 | 45736786 | 45765812 | chr7 | 56011066 | 56051604 | SEPT7P2_PSPH | − | − |
| chr7 | 50304668 | 50405101 | chr1 | 3069210 | 3438621 | IKZF1_PRDM16 | + | + |
| chr7 | 50304668 | 50405101 | chr12 | 11649853 | 11895402 | IKZF1_ETV6 | + | + |
| chr7 | 50304668 | 50405101 | chr12 | 55966768 | 55972784 | IKZF1_CDK2 | + | + |
| chr7 | 50304668 | 50405101 | chr15 | 34343314 | 34357737 | IKZF1_NUTM1 | + | + |
| chr7 | 50304668 | 50405101 | chr17 | 16415541 | 16437003 | IKZF1_TRPV2 | + | + |
| chr7 | 50304668 | 50405101 | chr3 | 9397718 | 9478154 | IKZF1_SETD5 | + | + |
| chr7 | 50304668 | 50405101 | chr7 | 48171459 | 48647495 | IKZF1_ABCA13 | + | + |
| chr7 | 50590062 | 50793462 | chr7 | 3301447 | 4269000 | GRB10_SDK1 | − | + |
| chr7 | 66114604 | 66154561 | chr10 | 96593311 | 96720514 | CRCP_PIK3AP1 | + | − |
| chr7 | 74657684 | 74760692 | chr17 | 40309193 | 40356796 | GTF2I_RARA | + | + |
| chr7 | 75533299 | 75738947 | chr5 | 150113836 | 150155860 | HIP1_PDGFRB | − | − |
| chr7 | 92560792 | 92590393 | chr7 | 92604920 | 92833917 | FAM133B_CDK6 | − | − |
| chr7 | 92604920 | 92833917 | chr11 | 118436463 | 118526832 | CDK6_KMT2A | − | + |
| chr7 | 92604920 | 92833917 | chr14 | 36516416 | 36521149 | CDK6_NKX2-1 | − | − |
| chr7 | 92604920 | 92833917 | chr3 | 169084760 | 169663470 | CDK6_MECOM | − | − |
| chr7 | 92604920 | 92833917 | chr5 | 171309283 | 171312134 | CDK6_TLX3 | − | + |
| chr7 | 92604920 | 92833917 | chr7 | 27269356 | 27269678 | CDK6_RPL35P4 | − | − |
| chr7 | 99472892 | 99503650 | chr7 | 99493239 | 99500297 | ZNF789_ZNF394 | + | − |
| chr7 | 100852752 | 100867008 | chr6 | 135181314 | 135219171 | SLC12A9_MYB | + | + |
| chr7 | 101815903 | 102283958 | chr4 | 73436162 | 73456174 | CUX1_AFP | + | + |
| chr7 | 101815903 | 102283958 | chr7 | 149126415 | 149182802 | CUX1_ZNF398 | + | + |
| chr7 | 101815903 | 102283958 | chr8 | 38411138 | 38468834 | CUX1_FGFR1 | + | − |
| chr7 | 107923817 | 108003255 | chr7 | 116862472 | 116922049 | LAMB1_CAPZA2 | − | + |
| chr7 | 116862472 | 116922049 | chr19 | 58544468 | 58550716 | CAPZA2_TRIM28 | + | + |
| chr7 | 138460333 | 138589993 | chr8 | 38411138 | 38468834 | TRIM24_FGFR1 | + | − |
| chr7 | 139043519 | 139109648 | chr13 | 42272152 | 42323267 | ZC3HAV1_AKAP11 | − | + |
| chr7 | 142299010 | 142813287 | chr10 | 101130504 | 101137789 | TRB_TLX1 | + | + |
| chr7 | 142299010 | 142813287 | chr11 | 8224313 | 8268716 | TRB_LMO1 | + | − |
| chr7 | 142299010 | 142813287 | chr14 | 95709966 | 95714196 | TRB_TCL1A | + | − |
| chr7 | 142299010 | 142813287 | chr22 | 37125837 | 37149990 | TRB_IL2RB | + | − |
| chr7 | 142299010 | 142813287 | chr3 | 169084760 | 169663470 | TRB_MECOM | + | − |
| chr7 | 142299010 | 142813287 | chr7 | 27162434 | 27165530 | TRB_HOXA9 | + | − |
| chr7 | 142299010 | 142813287 | chr7 | 27181514 | 27185216 | TRB_HOXA11 | + | − |
| chr7 | 142299010 | 142813287 | chr8 | 127736068 | 127741434 | TRB_MYC | + | + |
| chr7 | 142299010 | 142813287 | chrX | 108732481 | 108736409 | TRB_IRS4 | + | − |
| chr7 | 148697913 | 148800582 | chr7 | 153887096 | 154894285 | CUL1_DPP6 | + | + |
| chr7 | 152134924 | 152436005 | chr7 | 152759748 | 152855378 | KMT2C_ACTR3B | − | + |
| chr7 | 156994050 | 157009075 | chr12 | 11649853 | 11895402 | MNX1_ETV6 | − | + |
| chr7 | 158730994 | 158829628 | chr20 | 3471039 | 3651122 | ESYT2_ATRN | − | + |
| chr8 | 17922856 | 18029944 | chr9 | 4985244 | 5128183 | PCM1_JAK2 | + | + |
| chr8 | 23385782 | 23457695 | chr8 | 18527300 | 19013686 | ENTPD4_PSD3 | − | − |
| chr8 | 28890403 | 29053270 | chr9 | 4985244 | 5128183 | HMBOX1_JAK2 | + | + |
| chr8 | 38411138 | 38468834 | chr5 | 179820758 | 179838078 | FGFR1_SQSTM1 | − | + |
| chr8 | 38411138 | 38468834 | chr7 | 101815903 | 102283958 | FGFR1_CUX1 | − | + |
| chr8 | 38411138 | 38468834 | chr8 | 38411138 | 38468834 | FGFR1_FGFR1 | − | − |
| chr8 | 41929478 | 42052026 | chr16 | 3725053 | 3880726 | KAT6A_CREBBP | − | − |
| chr8 | 41929478 | 42052026 | chr19 | 39776594 | 39786135 | KAT6A_LEUTX | − | + |
| chr8 | 41929478 | 42052026 | chr2 | 25733752 | 25878516 | KAT6A_ASXL2 | − | − |
| chr8 | 41929478 | 42052026 | chr20 | 47501901 | 47656877 | KAT6A_NCOA3 | − | + |
| chr8 | 41929478 | 42052026 | chr22 | 41091785 | 41180079 | KAT6A_EP300 | − | + |
| chr8 | 41929478 | 42052026 | chr8 | 41929478 | 42052026 | KAT6A_KAT6A | − | − |
| chr8 | 41929478 | 42052026 | chr8 | 70109761 | 70403805 | KAT6A_NCOA2 | − | − |
| chr8 | 42338454 | 42371808 | chr8 | 132571952 | 132675617 | POLB_LRRC6 | + | − |
| chr8 | 42896931 | 43030539 | chr8 | 38411138 | 38468834 | HOOK3_FGFR1 | + | − |
| chr8 | 51817574 | 51899186 | chr7 | 549196 | 727341 | PCMTD1_PRKAR1B | − | − |
| chr8 | 56160903 | 56211279 | chr7 | 142797455 | 142797502 | PLAG1_TRBJ2-7 | − | + |
| chr8 | 99013265 | 99877580 | chrX | 123961313 | 124102656 | VPS13B_STAG2 | + | + |
| chr8 | 123219956 | 123241398 | chr7 | 27184670 | 27189169 | C8orf76_HOXA11-AS | − | + |
| chr8 | 127736068 | 127741434 | chr6 | 44828731 | 45377933 | MYC_SUPT3H | + | − |
| chr8 | 127736068 | 127741434 | chr9 | 37120538 | 37358149 | MYC_ZCCHC7 | + | + |
| chr8 | 127736068 | 127741434 | chr9 | 37438113 | 37465399 | MYC_ZBTB5 | + | − |
| chr8 | 127890627 | 128101253 | chr13 | 34942286 | 35672735 | PVT1_NBEA | + | + |
| chr8 | 127890627 | 128101253 | chr3 | 169084760 | 169663470 | PVT1_MECOM | + | − |
| chr8 | 127890627 | 128101253 | chr8 | 125091852 | 125367120 | PVT1_NSMCE2 | + | + |
| chr8 | 127890627 | 128101253 | chr8 | 130052106 | 130443660 | PVT1_ASAP1 | + | − |
| chr8 | 127890627 | 128101253 | chr9 | 37120538 | 37358149 | PVT1_ZCCHC7 | + | + |
| chr8 | 143833720 | 143840974 | chr9 | 109015151 | 109119945 | NRBP2_TMEM245 | − | − |
| chr9 | 470290 | 746105 | chr5 | 150113836 | 150155860 | KANK1_PDGFRB | + | − |
| chr9 | 2015185 | 2193620 | chr12 | 6666476 | 6689510 | SMARCA2_ZNF384 | + | − |
| chr9 | 2015185 | 2193620 | chr9 | 2621833 | 2660053 | SMARCA2_VLDLR | + | + |
| chr9 | 3218301 | 3526001 | chr9 | 4985244 | 5128183 | RFX3_JAK2 | − | + |
| chr9 | 4985244 | 5128183 | chr16 | 11976737 | 12574289 | JAK2_SNX29 | + | + |
| chr9 | 4985244 | 5128183 | chr5 | 158695919 | 159099761 | JAK2_EBF1 | + | − |
| chr9 | 15464065 | 15511019 | chr11 | 3675009 | 3797792 | PSIP1_NUP98 | − | − |
| chr9 | 21968104 | 21995301 | chr14 | 21621903 | 22552132 | CDKN2A_TRA | − | + |
| chr9 | 21968104 | 21995301 | chr9 | 21455483 | 21456049 | CDKN2A_IFNWP19 | − | + |
| chr9 | 21968104 | 21995301 | chr9 | 21802648 | 21937651 | CDKN2A_MTAP | − | + |
| chr9 | 33041763 | 33076659 | chr9 | 4985244 | 5128183 | SMU1_JAK2 | − | + |
| chr9 | 34179012 | 34252523 | chr9 | 34086386 | 34126773 | UBAP1_DCAF12 | + | − |
| chr9 | 35812959 | 35815021 | chr9 | 35792153 | 35809732 | HINT2_NPR2 | − | + |
| chr9 | 36833274 | 37034185 | chr10 | 7818503 | 8016627 | PAX5_TAF3 | − | + |
| chr9 | 36833274 | 37034185 | chr11 | 33542274 | 33674102 | PAX5_KIAA1549L | − | + |
| chr9 | 36833274 | 37034185 | chr12 | 132489550 | 132585188 | PAX5_FBRSL1 | − | + |
| chr9 | 36833274 | 37034185 | chr14 | 76310711 | 76498475 | PAX5_ESRRB | − | + |
| chr9 | 36833274 | 37034185 | chr16 | 88874857 | 88977204 | PAX5_CBFA2T3 | − | − |
| chr9 | 36833274 | 37034185 | chr16 | 89720984 | 89740903 | PAX5_ZNF276 | − | + |
| chr9 | 36833274 | 37034185 | chr17 | 47896149 | 47928957 | PAX5_SP2 | − | + |
| chr9 | 36833274 | 37034185 | chr20 | 32277663 | 32335011 | PAX5_KIF3B | − | + |
| chr9 | 36833274 | 37034185 | chr20 | 32358329 | 32439319 | PAX5_ASXL1 | − | + |
| chr9 | 36833274 | 37034185 | chr20 | 32443061 | 32584890 | PAX5_NOL4L | − | − |
| chr9 | 36833274 | 37034185 | chr20 | 46060984 | 46089952 | PAX5_NCOA5 | − | − |
| chr9 | 36833274 | 37034185 | chr3 | 152268039 | 152465779 | PAX5_MBNL1 | − | + |
| chr9 | 36833274 | 37034185 | chr6 | 10747830 | 10759774 | PAX5_TMEM14B | − | + |
| chr9 | 36833274 | 37034185 | chr7 | 86643913 | 86864884 | PAX5_GRM3 | − | + |
| chr9 | 36833274 | 37034185 | chr9 | 470290 | 746105 | PAX5_KANK1 | − | + |
| chr9 | 36833274 | 37034185 | chr9 | 20341664 | 20622543 | PAX5_MLLT3 | − | − |
| chr9 | 36833274 | 37034185 | chr9 | 36336403 | 36401198 | PAX5_RNF38 | − | − |
| chr9 | 36833274 | 37034185 | chr9 | 37120538 | 37358149 | PAX5_ZCCHC7 | − | + |
| chr9 | 36833274 | 37034185 | chr9 | 113165519 | 113221361 | PAX5_FKBP15 | − | − |
| chr9 | 36833274 | 37034185 | chrX | 1462571 | 1537107 | PAX5_P2RY8 | − | − |
| chr9 | 36833274 | 37034185 | chrX | 40051245 | 40177329 | PAX5_BCOR | − | − |
| chr9 | 36833274 | 37034185 | chrX | 120158560 | 120165630 | PAX5_RHOXF2 | − | + |
| chr9 | 37120538 | 37358149 | chr20 | 44496223 | 44522085 | ZCCHC7_SERINC3 | + | − |
| chr9 | 37120538 | 37358149 | chr9 | 36833274 | 37034185 | ZCCHC7_PAX5 | + | − |
| chr9 | 37422665 | 37436990 | chr19 | 6677703 | 6720682 | GRHPR_C3 | + | − |
| chr9 | 37919133 | 38069211 | chr11 | 118747765 | 118791136 | SHB_DDX6 | − | − |
| chr9 | 71683365 | 71768884 | chr8 | 27311481 | 27459390 | CEMIP2_PTK2B | − | + |
| chr9 | 92711362 | 92764812 | chr9 | 4985244 | 5128183 | BICD2_JAK2 | − | + |
| chr9 | 95875700 | 95968840 | chr19 | 41330322 | 41353911 | ERCC6L2_TGFB1 | + | − |
| chr9 | 100302083 | 100352939 | chr9 | 97674908 | 97697357 | TEX10_XPA | − | − |
| chr9 | 105662456 | 105663112 | chr7 | 142299010 | 142813287 | TAL2_TRB | + | + |
| chr9 | 109640787 | 109946703 | chr9 | 110048695 | 110172512 | PALM2_AKAP2 | + | + |
| chr9 | 111525158 | 111577844 | chr9 | 125261831 | 125367207 | ZNF483_GAPVD1 | + | + |
| chr9 | 111896765 | 111935369 | chr8 | 127890627 | 128101253 | UGCG_PVT1 | + | + |
| chr9 | 120388868 | 120580170 | chr4 | 54229096 | 54298247 | CDK5RAP2_PDGFRA | − | + |
| chr9 | 121075011 | 121177608 | chr4 | 54657917 | 54740715 | CNTRL_KIT | + | + |
| chr9 | 121075011 | 121177608 | chr8 | 38411138 | 38468834 | CNTRL_FGFR1 | + | − |
| chr9 | 123379653 | 123930107 | chr9 | 37120538 | 37358149 | DENND1A_ZCCHC7 | − | + |
| chr9 | 124517274 | 124771277 | chr9 | 133205279 | 133209250 | NR6A1_OBP2B | − | − |
| chr9 | 128683654 | 128696400 | chr9 | 131125560 | 131234670 | SET_NUP214 | + | + |
| chr9 | 129887186 | 130043194 | chr11 | 118436463 | 118526832 | FNBP1_KMT2A | − | + |
| chr9 | 130713945 | 130885683 | chr17 | 74748651 | 74769353 | ABL1_SLC9A3R1 | + | + |
| chr9 | 130713945 | 130885683 | chr22 | 23180209 | 23318037 | ABL1_BCR | + | + |
| chr9 | 130713945 | 130885683 | chr9 | 131394092 | 131500197 | ABL1_PRRC2B | + | + |
| chr9 | 131125560 | 131234670 | chr22 | 16783411 | 16821699 | NUP214_XKR3 | + | − |
| chr9 | 131125560 | 131234670 | chr7 | 6374522 | 6403977 | NUP214_RAC1 | + | + |
| chr9 | 131125560 | 131234670 | chr9 | 130713945 | 130885683 | NUP214_ABL1 | + | + |
| chr9 | 131394092 | 131500197 | chr10 | 129467183 | 129768007 | PRRC2B_MGMT | + | + |
| chr9 | 131394092 | 131500197 | chr22 | 23180209 | 23318037 | PRRC2B_BCR | + | + |
| chr9 | 136440096 | 136483759 | chr9 | 136494432 | 136545786 | SEC16A_NOTCH1 | − | − |
| chr9 | 136494432 | 136545786 | chr9 | 136862118 | 136866286 | NOTCH1_EDF1 | − | − |
| chr9 | 136658855 | 136672678 | chr17 | 83079690 | 83095119 | EGFL7_METRNL | + | + |
| chr9 | 136862118 | 136866286 | chr9 | 136494432 | 136545786 | EDF1_NOTCH1 | − | − |
| chrX | 1462571 | 1537107 | chr9 | 36833274 | 37034185 | P2RY8_PAX5 | − | − |
| chrX | 2691132 | 2741309 | chr9 | 4985244 | 5128183 | CD99_JAK2 | + | + |
| chrX | 13734744 | 13769353 | chr9 | 4985244 | 5128183 | OFD1_JAK2 | + | + |
| chrX | 40051245 | 40177329 | chr17 | 40309193 | 40356796 | BCOR_RARA | − | + |
| chrX | 49028730 | 49043486 | chrX | 71283191 | 71301166 | TFE3_NONO | − | + |
| chrX | 55075062 | 55078909 | chrX | 55009054 | 55031064 | PAGE2B_ALAS2 | + | − |
| chrX | 71118555 | 71142453 | chr7 | 27162434 | 27165530 | MED12_HOXA9 | + | − |
| chrX | 71283191 | 71301166 | chr2 | 43222401 | 43226609 | NONO_ZFP36L2 | + | − |
| chrX | 71283191 | 71301166 | chrX | 49028730 | 49043486 | NONO_TFE3 | + | − |
| chrX | 100969710 | 100990806 | chrX | 101009345 | 101052116 | ARL13A_TRMT2B | + | − |
| chrX | 123859811 | 123913976 | chr12 | 10158300 | 10172138 | XIAP_OLR1 | + | − |
| chrX | 123961313 | 124102656 | chr7 | 16646130 | 16706523 | STAG2_BZW2 | + | + |
| chrX | 123961313 | 124102656 | chrX | 77504877 | 77786216 | STAG2_ATRX | + | − |
| chrX | 123961313 | 124102656 | chrX | 130384439 | 130385447 | STAG2_GPR119 | + | − |
| chrX | 130064873 | 130110716 | chr21 | 38380029 | 38661780 | ELF4_ERG | − | − |
| chrX | 138632677 | 139205023 | chr6 | 134169245 | 134318058 | FGF13_SGK1 | − | − |

Gene-Level Variant Identification

Gene mutations are identified in—85 kbp targeting 40 genes and gene hotspots that are recurrently mutated in AML or MDS8. This target space was selected to be identical to that of the targeted gene panel used for clinical testing of these patients at our institution, and is relatively small so that rare inherited variants (i.e., variants of uncertain significance (VUS)), are minimized. The primary variant caller is Varscan2, which is run in SNV and indel mode using custom parameters to enhance sensitivity. The indel caller Pindel and Manta are also run on exons 13-15 of FLT3 to identify FLT3 ITD alleles. In addition, a read count based ‘hotspot’ analysis is performed on 66 recurrently mutated positions to recover low abundance variants that are not detected by Varscan2 (a minimum variant read count of 3 is required to report these hotspot positions). Variant calls identified via these approaches are merged and harmonized using a custom python script (available upon request), and annotated with VEP using Ensembl version 90 prior to reporting.

Report Generation

Annotated CNA, SV, and gene mutation calls are combined with coverage QC information to generate a final text report using a custom python script (available upon request). This report includes the CNAs, recurrent SVs, and gene mutations identified by the above steps as ‘toplevel’ results. The remaining SVs that remained after filtering are reported in two categories. The first includes high-quality novel SVs that affect (overlap) a gene that is included in either the recurrent SV or gene mutation target space. The second category is all other high-quality novel SVs. Additional coverage QC metrics are also reported. This text file is copied to the final case directory along with data files (CRAM, and VCF) and graphical coverage plots from ichorCNA. The final text report is also used to generate a graphical ChromoSeq report, as shown in FIG. 9.

WGS Analysis for Study Patients

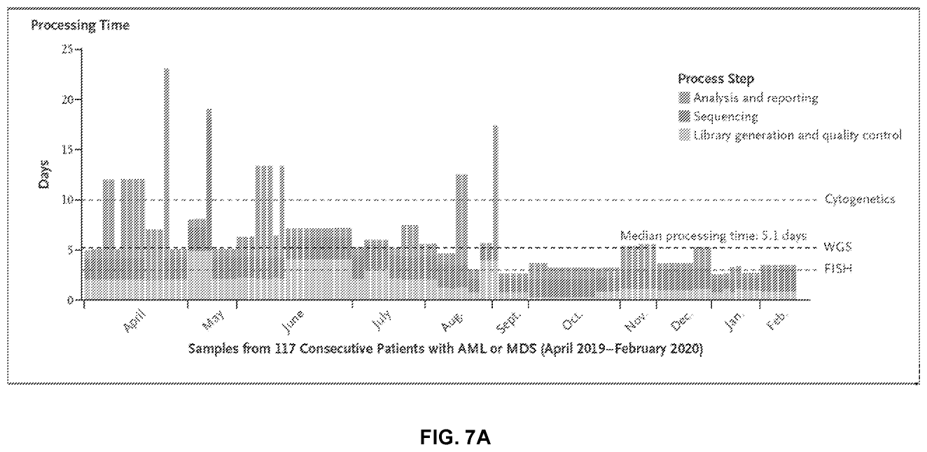

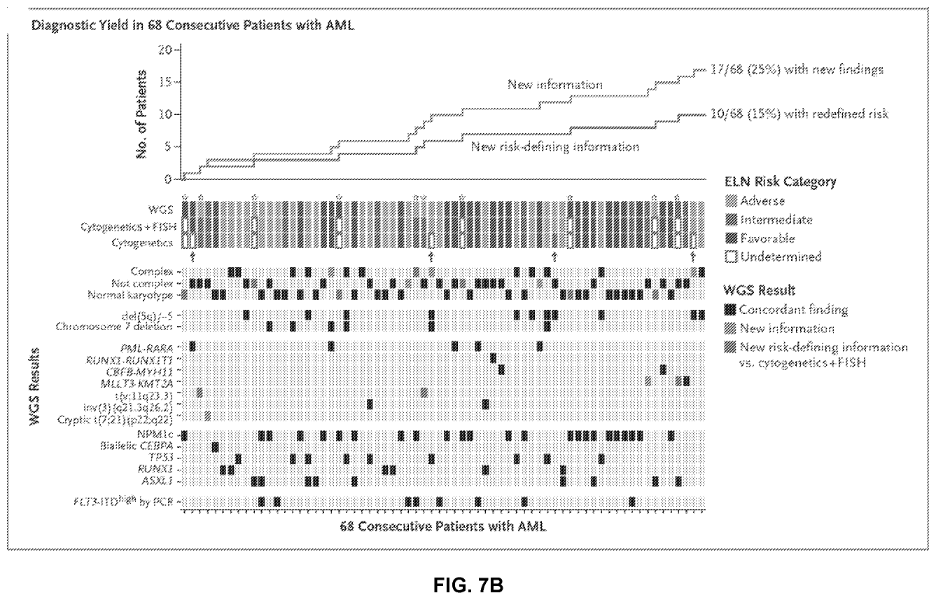

All retrospective samples were sequenced on S4 flowcells and processed using in-house demultiplexing, aligned with the local DRAGEN server, and analyzed on a local compute cluster with the ChromoSeq workflow. Prospective samples were sequenced on S1 flowcells and initially analyzed using the cloud-based approach on BaseSpace to record failure rates and turnaround times. ChromoSeq reports with QC metrics and variants for the prospective patients were reviewed in 1 hour sessions by board-certified molecular pathologists and a board-certified cytogeneticist and molecular geneticist without prior knowledge of the results from conventional testing. Exact times for processing steps shown in FIG. 7A were obtained from the MGI LIMS system and from the timestamp in the ChromoSeq text report. A final ChromoSeq analysis was performed on all prospective samples at the end of the study to harmonize the results and file formats (which changed over the course of the study) with the outputs from the retrospective samples.

Conventional Cytogenetic and Molecular Analysis

All cytogenetic and FISH analyses were performed according to standard clinical protocols. We obtained data regarding genetic mutations as part of standard diagnostic testing using polymerase-chain-reaction (PCR)—based assays for the internal tandem duplication mutation in FLT3 (FLT3-ITD) and the NPM1c mutation, a laboratory-developed clinical sequencing assay, or both. Cytogenetic and molecular results were used to assign patients to established European Leukemia Network (ELN) or IPSS-R risk categories.

Culture of cells from bone marrow or leukemic peripheral blood samples was performed per standard clinical protocols, followed by harvest, slide dropping, G-banding with trypsin/Wright stain, and analysis. Cytogenetic events were considered clonal if they occurred in at least two metaphases (at least three metaphases for monosomies). For the purposes of this study, cytogenetic analysis was called ‘unsuccessful’ if no metaphases were obtained for analysis, and ‘inconclusive’ if fewer than 20 metaphases were analyzed without detection of clonal abnormalities, which is similar to approaches taken by other studies. FISH results used for risk stratification and calculation of the yield of WGS in the prospective cohort were obtained from clinical reports performed at diagnosis. For AML patients, most FISH studies (60 of 68 patients) included the ELN-recommended panel of PML-RARA (LSI PML/RARA Dual Color, Dual Fusion, Abbot/Vysis) CBFB-MYH11 (LSI CBFB Dual Color, Break Apart Rearrangement Probe, Abbot/Vysis), RUNX1-RUNX1T1 (LSI RUNX1T1/RUNX1 Dual Color, Dual Fusion, Vysis), del(5q) (D5S630/D5S2064 Dual Color Probe, Cytocell/Aquarius), and del(7q) (LSI D7S486/D7Z1 Dual Color Probe, Abbot/Vysis). Additional FISH assays were also performed to confirm WGS findings but were not used to for risk group assignments.

Gene mutations were obtained as part of standard diagnostic testing using a commercially available PCR-based assay for the FLT3 internal tandem duplication mutation (ITD) (Invivoscribe, San Diego, Calif.), in-house testing for the NPM1c mutation, and/or a laboratory-developed clinical sequencing assay, including either clinical tumor/normal exome sequencing or a clinical gene panel that targets 40 recurrently mutated genes or gene hotspots in AML and MDS (Myeloseq; Department of Pathology and Immunology, Washington University School of Medicine, see Table

TABLE S1
Target list for gene mutation identification

| Chrom | Start | End | Exon | Strand | Gene Name | GeneID | TranscriptID |
|---|---|---|---|---|---|---|---|
| chr1 | 114713797 | 114713981 | NRAS_exon_3 | − | NRAS | ENSG00000213281 | ENST00000369535 |
| chr1 | 114716047 | 114716163 | NRAS_exon_2 | − | NRAS | ENSG00000213281 | ENST00000369535 |
| chr1 | 36466356 | 36466911 | CSF3R_exon_17 | − | CSF3R | ENSG00000119535 | ENST00000373103 |
| chr1 | 36467226 | 36467314 | CSF3R_exon_16 | − | CSF3R | ENSG00000119535 | ENST00000373103 |
| chr1 | 36467554 | 36467654 | CSF3R_exon_15 | − | CSF3R | ENSG00000119535 | ENST00000373103 |
| chr1 | 36467818 | 36467965 | CSF3R_exon_14 | − | CSF3R | ENSG00000119535 | ENST00000373103 |
| chr1 | 36468071 | 36468224 | CSF3R_exon_13 | − | CSF3R | ENSG00000119535 | ENST00000373103 |
| chr1 | 36469152 | 36469260 | CSF3R_exon_12 | − | CSF3R | ENSG00000119535 | ENST00000373103 |
| chr1 | 36469648 | 36469843 | CSF3R_exon_11 | − | CSF3R | ENSG00000119535 | ENST00000373103 |
| chr1 | 36471429 | 36471649 | CSF3R_exon_10 | − | CSF3R | ENSG00000119535 | ENST00000373103 |
| chr1 | 36472062 | 36472142 | CSF3R_exon_9 | − | CSF3R | ENSG00000119535 | ENST00000373103 |
| chr1 | 36472234 | 36472394 | CSF3R_exon_8 | − | CSF3R | ENSG00000119535 | ENST00000373103 |
| chr1 | 36472513 | 36472689 | CSF3R_exon_7 | − | CSF3R | ENSG00000119535 | ENST00000373103 |
| chr1 | 36473431 | 36473625 | CSF3R_exon_6 | − | CSF3R | ENSG00000119535 | ENST00000373103 |
| chr1 | 36473760 | 36473890 | CSF3R_exon_5 | − | CSF3R | ENSG00000119535 | ENST00000373103 |
| chr1 | 36475373 | 36475676 | CSF3R_exon_4 | − | CSF3R | ENSG00000119535 | ENST00000373103 |
| chr1 | 36479429 | 36479499 | CSF3R_exon_3 | − | CSF3R | ENSG00000119535 | ENST00000373103 |
| chr1 | 43349260 | 43349362 | MPL_exon_10 | + | MPL | ENSG00000117400 | ENST00000372470 |
| chr10 | 110567813 | 110567834 | SMC3_exon_1 | + | SMC3 | ENSG00000108055 | ENST00000361804 |
| chr10 | 110568934 | 110569016 | SMC3_exon_2 | + | SMC3 | ENSG00000108055 | ENST00000361804 |
| chr10 | 110573703 | 110573748 | SMC3_exon_3 | + | SMC3 | ENSG00000108055 | ENST00000361804 |
| chr10 | 110575332 | 110575406 | SMC3_exon_4 | + | SMC3 | ENSG00000108055 | ENST00000361804 |
| chr10 | 110577417 | 110577495 | SMC3_exon_5 | + | SMC3 | ENSG00000108055 | ENST00000361804 |
| chr10 | 110577831 | 110577917 | SMC3_exon_6 | + | SMC3 | ENSG00000108055 | ENST00000361804 |
| chr10 | 110578624 | 110578709 | SMC3_exon_7 | + | SMC3 | ENSG00000108055 | ENST00000361804 |
| chr10 | 110580900 | 110581024 | SMC3_exon_8 | + | SMC3 | ENSG00000108055 | ENST00000361804 |
| chr10 | 110581919 | 110582101 | SMC3_exon_9 | + | SMC3 | ENSG00000108055 | ENST00000361804 |
| chr10 | 110582558 | 110582645 | SMC3_exon_10 | + | SMC3 | ENSG00000108055 | ENST00000361804 |
| chr10 | 110583380 | 110583551 | SMC3_exon_11 | + | SMC3 | ENSG00000108055 | ENST00000361804 |
| chr10 | 110583837 | 110583965 | SMC3_exon_12 | + | SMC3 | ENSG00000108055 | ENST00000361804 |
| chr10 | 110584179 | 110584399 | SMC3_exon_13 | + | SMC3 | ENSG00000108055 | ENST00000361804 |
| chr10 | 110589601 | 110589711 | SMC3_exon_14 | + | SMC3 | ENSG00000108055 | ENST00000361804 |
| chr10 | 110589888 | 110589994 | SMC3_exon_15 | + | SMC3 | ENSG00000108055 | ENST00000361804 |
| chr10 | 110590408 | 110590575 | SMC3_exon_16 | + | SMC3 | ENSG00000108055 | ENST00000361804 |
| chr10 | 110590987 | 110591135 | SMC3_exon_17 | + | SMC3 | ENSG00000108055 | ENST00000361804 |
| chr10 | 110593069 | 110593226 | SMC3_exon_18 | + | SMC3 | ENSG00000108055 | ENST00000361804 |
| chr10 | 110596394 | 110596553 | SMC3_exon_19 | + | SMC3 | ENSG00000108055 | ENST00000361804 |
| chr10 | 110598135 | 110598293 | SMC3_exon_20 | + | SMC3 | ENSG00000108055 | ENST00000361804 |
| chr10 | 110599650 | 110599815 | SMC3_exon_21 | + | SMC3 | ENSG00000108055 | ENST00000361804 |
| chr10 | 110600435 | 110600549 | SMC3_exon_22 | + | SMC3 | ENSG00000108055 | ENST00000361804 |
| chr10 | 110601018 | 110601133 | SMC3_exon_23 | + | SMC3 | ENSG00000108055 | ENST00000361804 |
| chr10 | 110601633 | 110601887 | SMC3_exon_24 | + | SMC3 | ENSG00000108055 | ENST00000361804 |
| chr10 | 110601962 | 110602181 | SMC3_exon_25 | + | SMC3 | ENSG00000108055 | ENST00000361804 |
| chr10 | 110602470 | 110602668 | SMC3_exon_26 | + | SMC3 | ENSG00000108055 | ENST00000361804 |
| chr10 | 110602821 | 110603005 | SMC3_exon_27 | + | SMC3 | ENSG00000108055 | ENST00000361804 |
| chr10 | 110603180 | 110603293 | SMC3_exon_28 | + | SMC3 | ENSG00000108055 | ENST00000361804 |
| chr10 | 110604227 | 110604302 | SMC3_exon_29 | + | SMC3 | ENSG00000108055 | ENST00000361804 |
| chr11 | 119278163 | 119278300 | CBL_exon_8 | + | CBL | ENSG00000110395 | ENST00000264033 |
| chr11 | 119278507 | 119278716 | CBL_exon_9 | + | CBL | ENSG00000110395 | ENST00000264033 |
| chr11 | 32389057 | 32389182 | WT1_exon_10 | − | WT1 | ENSG00000184937 | ENST00000332351 |
| chr11 | 32391968 | 32392067 | WT1_exon_9 | − | WT1 | ENSG00000184937 | ENST00000332351 |
| chr11 | 32392662 | 32392758 | WT1_exon_8 | − | WT1 | ENSG00000184937 | ENST00000332351 |
| chr11 | 32396253 | 32396410 | WT1_exon_7 | − | WT1 | ENSG00000184937 | ENST00000332351 |
| chr11 | 32399944 | 32400047 | WT1_exon_6 | − | WT1 | ENSG00000184937 | ENST00000332351 |
| chr11 | 32416486 | 32416543 | WT1_exon_5 | − | WT1 | ENSG00000184937 | ENST00000332351 |
| chr11 | 32417573 | 32417657 | WT1_exon_4 | − | WT1 | ENSG00000184937 | ENST00000332351 |
| chr11 | 32427952 | 32428061 | WT1_exon_3 | − | WT1 | ENSG00000184937 | ENST00000332351 |
| chr11 | 32428493 | 32428622 | WT1_exon_2 | − | WT1 | ENSG00000184937 | ENST00000332351 |
| chr11 | 32434696 | 32435348 | WT1_exon_1 | − | WT1 | ENSG00000184937 | ENST00000332351 |
| chr12 | 112450315 | 112450515 | PTPN11_exon_3 | + | PTPN11 | ENSG00000179295 | ENST00000351677 |
| chr12 | 112489021 | 112489178 | PTPN11_exon_13 | + | PTPN11 | ENSG00000179295 | ENST00000351677 |
| chr12 | 112502141 | 112502259 | PTPN11_exon_14 | + | PTPN11 | ENSG00000179295 | ENST00000351677 |
| chr12 | 11650124 | 11650163 | ETV6_exon_1 | + | ETV6 | ENSG00000139083 | ENST00000396373 |
| chr12 | 11752446 | 11752582 | ETV6_exon_2 | + | ETV6 | ENSG00000139083 | ENST00000396373 |
| chr12 | 11839136 | 11839307 | ETV6_exon_3 | + | ETV6 | ENSG00000139083 | ENST00000396373 |
| chr12 | 11853423 | 11853564 | ETV6_exon_4 | + | ETV6 | ENSG00000139083 | ENST00000396373 |
| chr12 | 11869420 | 11869972 | ETV6_exon_5 | + | ETV6 | ENSG00000139083 | ENST00000396373 |
| chr12 | 11884441 | 11884590 | ETV6_exon_6 | + | ETV6 | ENSG00000139083 | ENST00000396373 |
| chr12 | 11885922 | 11886029 | ETV6_exon_7 | + | ETV6 | ENSG00000139083 | ENST00000396373 |
| chr12 | 11890937 | 11891046 | ETV6_exon_8 | + | ETV6 | ENSG00000139083 | ENST00000396373 |
| chr12 | 25227231 | 25227415 | KRAS_exon_3 | − | KRAS | ENSG00000133703 | ENST00000256078 |
| chr12 | 25245271 | 25245387 | KRAS_exon_2 | − | KRAS | ENSG00000133703 | ENST00000256078 |
| chr13 | 28018463 | 28018592 | FLT3_exon_20 | − | FLT3 | ENSG00000122025 | ENST00000241453 |
| chr13 | 28033884 | 28033994 | FLT3_exon_15 | − | FLT3 | ENSG00000122025 | ENST00000241453 |
| chr13 | 28034079 | 28034217 | FLT3_exon_14 | − | FLT3 | ENSG00000122025 | ENST00000241453 |
| chr13 | 28034298 | 28034410 | FLT3_exon_13 | − | FLT3 | ENSG00000122025 | ENST00000241453 |
| chr15 | 90088584 | 90088750 | IDH2_exon_4 | − | IDH2 | ENSG00000182054 | ENST00000330062 |
| chr17 | 31095306 | 31095372 | NF1_exon_1 | + | NF1 | ENSG00000196712 | ENST00000358273 |
| chr17 | 31155979 | 31156129 | NF1_exon_2 | + | NF1 | ENSG00000196712 | ENST00000358273 |
| chr17 | 31159006 | 31159096 | NF1_exon_3 | + | NF1 | ENSG00000196712 | ENST00000358273 |
| chr17 | 31163182 | 31163379 | NF1_exon_4 | + | NF1 | ENSG00000196712 | ENST00000358273 |
| chr17 | 31169887 | 31170000 | NF1_exon_5 | + | NF1 | ENSG00000196712 | ENST00000358273 |
| chr17 | 31181418 | 31181492 | NF1_exon_6 | + | NF1 | ENSG00000196712 | ENST00000358273 |
| chr17 | 31181706 | 31181788 | NF1_exon_7 | + | NF1 | ENSG00000196712 | ENST00000358273 |
| chr17 | 31182504 | 31182668 | NF1_exon_8 | + | NF1 | ENSG00000196712 | ENST00000358273 |
| chr17 | 31200418 | 31200598 | NF1_exon_9 | + | NF1 | ENSG00000196712 | ENST00000358273 |
| chr17 | 31201033 | 31201162 | NF1_exon_10 | + | NF1 | ENSG00000196712 | ENST00000358273 |
| chr17 | 31201407 | 31201488 | NF1_exon_11 | + | NF1 | ENSG00000196712 | ENST00000358273 |
| chr17 | 31206236 | 31206374 | NF1_exon_12 | + | NF1 | ENSG00000196712 | ENST00000358273 |
| chr17 | 31214447 | 31214588 | NF1_exon_13 | + | NF1 | ENSG00000196712 | ENST00000358273 |
| chr17 | 31219001 | 31219121 | NF1_exon_14 | + | NF1 | ENSG00000196712 | ENST00000358273 |
| chr17 | 31221846 | 31221932 | NF1_exon_15 | + | NF1 | ENSG00000196712 | ENST00000358273 |
| chr17 | 31223440 | 31223570 | NF1_exon_16 | + | NF1 | ENSG00000196712 | ENST00000358273 |
| chr17 | 31225091 | 31225253 | NF1_exon_17 | + | NF1 | ENSG00000196712 | ENST00000358273 |
| chr17 | 31226431 | 31226687 | NF1_exon_18 | + | NF1 | ENSG00000196712 | ENST00000358273 |
| chr17 | 31227214 | 31227294 | NF1_exon_19 | + | NF1 | ENSG00000196712 | ENST00000358273 |
| chr17 | 31227519 | 31227609 | NF1_exon_20 | + | NF1 | ENSG00000196712 | ENST00000358273 |
| chr17 | 31229021 | 31229468 | NF1_exon_21 | + | NF1 | ENSG00000196712 | ENST00000358273 |
| chr17 | 31229831 | 31229977 | NF1_exon_22 | + | NF1 | ENSG00000196712 | ENST00000358273 |
| chr17 | 31230256 | 31230385 | NF1_exon_23 | + | NF1 | ENSG00000196712 | ENST00000358273 |
| chr17 | 31230838 | 31230928 | NF1_exon_24 | + | NF1 | ENSG00000196712 | ENST00000358273 |
| chr17 | 31232069 | 31232192 | NF1_exon_25 | + | NF1 | ENSG00000196712 | ENST00000358273 |
| chr17 | 31232696 | 31232884 | NF1_exon_26 | + | NF1 | ENSG00000196712 | ENST00000358273 |
| chr17 | 31232998 | 31233216 | NF1_exon_27 | + | NF1 | ENSG00000196712 | ENST00000358273 |
| chr17 | 31235607 | 31235775 | NF1_exon_28 | + | NF1 | ENSG00000196712 | ENST00000358273 |
| chr17 | 31235914 | 31236024 | NF1_exon_29 | + | NF1 | ENSG00000196712 | ENST00000358273 |
| chr17 | 31248980 | 31249122 | NF1_exon_30 | + | NF1 | ENSG00000196712 | ENST00000358273 |
| chr17 | 31252934 | 31253003 | NF1_exon_31 | + | NF1 | ENSG00000196712 | ENST00000358273 |
| chr17 | 31258340 | 31258505 | NF1_exon_32 | + | NF1 | ENSG00000196712 | ENST00000358273 |
| chr17 | 31259028 | 31259132 | NF1_exon_33 | + | NF1 | ENSG00000196712 | ENST00000358273 |
| chr17 | 31260365 | 31260518 | NF1_exon_34 | + | NF1 | ENSG00000196712 | ENST00000358273 |
| chr17 | 31261707 | 31261860 | NF1_exon_35 | + | NF1 | ENSG00000196712 | ENST00000358273 |
| chr17 | 31265225 | 31265342 | NF1_exon_36 | + | NF1 | ENSG00000196712 | ENST00000358273 |
| chr17 | 31325816 | 31326255 | NF1_exon_37 | + | NF1 | ENSG00000196712 | ENST00000358273 |
| chr17 | 31327495 | 31327842 | NF1_exon_38 | + | NF1 | ENSG00000196712 | ENST00000358273 |
| chr17 | 31330292 | 31330501 | NF1_exon_39 | + | NF1 | ENSG00000196712 | ENST00000358273 |
| chr17 | 31334834 | 31335034 | NF1_exon_40 | + | NF1 | ENSG00000196712 | ENST00000358273 |
| chr17 | 31336329 | 31336476 | NF1_exon_41 | + | NF1 | ENSG00000196712 | ENST00000358273 |
| chr17 | 31336631 | 31336917 | NF1_exon_42 | + | NF1 | ENSG00000196712 | ENST00000358273 |
| chr17 | 31337364 | 31337585 | NF1_exon_43 | + | NF1 | ENSG00000196712 | ENST00000358273 |
| chr17 | 31337815 | 31337883 | NF1_exon_44 | + | NF1 | ENSG00000196712 | ENST00000358273 |
| chr17 | 31338021 | 31338142 | NF1_exon_45 | + | NF1 | ENSG00000196712 | ENST00000358273 |
| chr17 | 31338700 | 31338808 | NF1_exon_46 | + | NF1 | ENSG00000196712 | ENST00000358273 |
| chr17 | 31340501 | 31340648 | NF1_exon_47 | + | NF1 | ENSG00000196712 | ENST00000358273 |
| chr17 | 31343005 | 31343138 | NF1_exon_48 | + | NF1 | ENSG00000196712 | ENST00000358273 |
| chr17 | 31349116 | 31349254 | NF1_exon_49 | + | NF1 | ENSG00000196712 | ENST00000358273 |
| chr17 | 31350179 | 31350321 | NF1_exon_50 | + | NF1 | ENSG00000196712 | ENST00000358273 |
| chr17 | 31352253 | 31352417 | NF1_exon_51 | + | NF1 | ENSG00000196712 | ENST00000358273 |
| chr17 | 31356456 | 31356585 | NF1_exon_52 | + | NF1 | ENSG00000196712 | ENST00000358273 |
| chr17 | 31356956 | 31357093 | NF1_exon_53 | + | NF1 | ENSG00000196712 | ENST00000358273 |
| chr17 | 31357265 | 31357372 | NF1_exon_54 | + | NF1 | ENSG00000196712 | ENST00000358273 |
| chr17 | 31358476 | 31358625 | NF1_exon_55 | + | NF1 | ENSG00000196712 | ENST00000358273 |
| chr17 | 31358965 | 31359018 | NF1_exon_56 | + | NF1 | ENSG00000196712 | ENST00000358273 |
| chr17 | 31360483 | 31360706 | NF1_exon_57 | + | NF1 | ENSG00000196712 | ENST00000358273 |
| chr17 | 31374009 | 31374155 | NF1_exon_58 | + | NF1 | ENSG00000196712 | ENST00000358273 |
| chr17 | 31937243 | 31937523 | SUZ12_exon_1 | + | SUZ12 | ENSG00000178691 | ENST00000322652 |
| chr17 | 31940282 | 31940335 | SUZ12_exon_2 | + | SUZ12 | ENSG00000178691 | ENST00000322652 |
| chr17 | 31940418 | 31940489 | SUZ12_exon_3 | + | SUZ12 | ENSG00000178691 | ENST00000322652 |
| chr17 | 31947613 | 31947688 | SUZ12_exon_4 | + | SUZ12 | ENSG00000178691 | ENST00000322652 |
| chr17 | 31966143 | 31966199 | SUZ12_exon_5 | + | SUZ12 | ENSG00000178691 | ENST00000322652 |
| chr17 | 31973142 | 31973234 | SUZ12_exon_6 | + | SUZ12 | ENSG00000178691 | ENST00000322652 |
| chr17 | 31975478 | 31975716 | SUZ12_exon_7 | + | SUZ12 | ENSG00000178691 | ENST00000322652 |
| chr17 | 31976517 | 31976617 | SUZ12_exon_8 | + | SUZ12 | ENSG00000178691 | ENST00000322652 |
| chr17 | 31982995 | 31983107 | SUZ12_exon_9 | + | SUZ12 | ENSG00000178691 | ENST00000322652 |
| chr17 | 31988316 | 31988500 | SUZ12_exon_10 | + | SUZ12 | ENSG00000178691 | ENST00000322652 |
| chr17 | 31993238 | 31993336 | SUZ12_exon_11 | + | SUZ12 | ENSG00000178691 | ENST00000322652 |
| chr17 | 31993861 | 31994011 | SUZ12_exon_12 | + | SUZ12 | ENSG00000178691 | ENST00000322652 |
| chr17 | 31994560 | 31994724 | SUZ12_exon_13 | + | SUZ12 | ENSG00000178691 | ENST00000322652 |
| chr17 | 31995560 | 31995765 | SUZ12_exon_14 | + | SUZ12 | ENSG00000178691 | ENST00000322652 |
| chr17 | 31996794 | 31996880 | SUZ12_exon_15 | + | SUZ12 | ENSG00000178691 | ENST00000322652 |
| chr17 | 31998654 | 31999003 | SUZ12_exon_16 | + | SUZ12 | ENSG00000178691 | ENST00000322652 |
| chr17 | 60662991 | 60663552 | PPM1D_exon_6 | + | PPM1D | ENSG00000170836 | ENST00000305921 |
| chr17 | 7669608 | 7669693 | TP53_exon_11 | − | TP53 | ENSG00000141510 | ENST00000269305 |
| chr17 | 7670605 | 7670718 | TP53_exon_10 | − | TP53 | ENSG00000141510 | ENST00000269305 |
| chr17 | 7673531 | 7673611 | TP53_exon_9 | − | TP53 | ENSG00000141510 | ENST00000269305 |
| chr17 | 76736796 | 76737163 | SRSF2_exon_1 | − | SRSF2 | ENSG00000161547 | ENST00000392485 |
| chr17 | 7673697 | 7673840 | TP53_exon_8 | − | TP53 | ENSG00000141510 | ENST00000269305 |
| chr17 | 7674177 | 7674293 | TP53_exon_7 | − | TP53 | ENSG00000141510 | ENST00000269305 |
| chr17 | 7674855 | 7674974 | TP53_exon_6 | − | TP53 | ENSG00000141510 | ENST00000269305 |
| chr17 | 7675049 | 7675239 | TP53_exon_5 | − | TP53 | ENSG00000141510 | ENST00000269305 |
| chr17 | 7675990 | 7676275 | TP53_exon_4 | − | TP53 | ENSG00000141510 | ENST00000269305 |
| chr17 | 7676378 | 7676406 | TP53_exon_3 | − | TP53 | ENSG00000141510 | ENST00000269305 |
| chr17 | 7676517 | 7676597 | TP53_exon_2 | − | TP53 | ENSG00000141510 | ENST00000269305 |
| chr19 | 12943710 | 12943913 | CALR_exon_9 | + | CALR | ENSG00000179218 | ENST00000316448 |
| chr19 | 33301337 | 33302417 | CEBPA_exon_1 | − | CEBPA | ENSG00000245848 | ENST00000498907 |
| chr2 | 197392302 | 197392464 | SF3B1_exon_25 | − | SF3B1 | ENSG00000115524 | ENST00000335508 |
| chr2 | 197392968 | 197393191 | SF3B1_exon_24 | − | SF3B1 | ENSG00000115524 | ENST00000335508 |
| chr2 | 197396052 | 197396331 | SF3B1_exon_23 | − | SF3B1 | ENSG00000115524 | ENST00000335508 |
| chr2 | 197397981 | 197398119 | SF3B1_exon_22 | − | SF3B1 | ENSG00000115524 | ENST00000335508 |
| chr2 | 197398457 | 197398584 | SF3B1_exon_21 | − | SF3B1 | ENSG00000115524 | ENST00000335508 |
| chr2 | 197400051 | 197400169 | SF3B1_exon_20 | − | SF3B1 | ENSG00000115524 | ENST00000335508 |
| chr2 | 197400248 | 197400437 | SF3B1_exon_19 | − | SF3B1 | ENSG00000115524 | ENST00000335508 |
| chr2 | 197400711 | 197400939 | SF3B1_exon_18 | − | SF3B1 | ENSG00000115524 | ENST00000335508 |
| chr2 | 197401396 | 197401528 | SF3B1_exon_17 | − | SF3B1 | ENSG00000115524 | ENST00000335508 |
| chr2 | 197401738 | 197401891 | SF3B1_exon_16 | − | SF3B1 | ENSG00000115524 | ENST00000335508 |
| chr2 | 197401981 | 197402133 | SF3B1_exon_15 | − | SF3B1 | ENSG00000115524 | ENST00000335508 |
| chr2 | 197402552 | 197402829 | SF3B1_exon_14 | − | SF3B1 | ENSG00000115524 | ENST00000335508 |
| chr2 | 197402945 | 197403038 | SF3B1_exon_13 | − | SF3B1 | ENSG00000115524 | ENST00000335508 |
| chr2 | 197403581 | 197403767 | SF3B1_exon_12 | − | SF3B1 | ENSG00000115524 | ENST00000335508 |
| chr2 | 197405072 | 197405180 | SF3B1_exon_11 | − | SF3B1 | ENSG00000115524 | ENST00000335508 |
| chr2 | 197405271 | 197405475 | SF3B1_exon_10 | − | SF3B1 | ENSG00000115524 | ENST00000335508 |
| chr2 | 197407994 | 197408122 | SF3B1_exon_9 | − | SF3B1 | ENSG00000115524 | ENST00000335508 |
| chr2 | 197408365 | 197408584 | SF3B1_exon_8 | − | SF3B1 | ENSG00000115524 | ENST00000335508 |
| chr2 | 197409766 | 197410010 | SF3B1_exon_7 | − | SF3B1 | ENSG00000115524 | ENST00000335508 |
| chr2 | 197416737 | 197416914 | SF3B1_exon_6 | − | SF3B1 | ENSG00000115524 | ENST00000335508 |
| chr2 | 197418505 | 197418591 | SF3B1_exon_5 | − | SF3B1 | ENSG00000115524 | ENST00000335508 |
| chr2 | 197420424 | 197420545 | SF3B1_exon_4 | − | SF3B1 | ENSG00000115524 | ENST00000335508 |
| chr2 | 197421025 | 197421136 | SF3B1_exon_3 | − | SF3B1 | ENSG00000115524 | ENST00000335508 |
| chr2 | 197423804 | 197423977 | SF3B1_exon_2 | − | SF3B1 | ENSG00000115524 | ENST00000335508 |
| chr2 | 197434968 | 197435002 | SF3B1_exon_1 | − | SF3B1 | ENSG00000115524 | ENST00000335508 |
| chr2 | 208248366 | 208248663 | IDH1_exon_4 | − | IDH1 | ENSG00000138413 | ENST00000415913 |
| chr2 | 25234278 | 25234423 | DNMT3A_exon_23 | − | DNMT3A | ENSG00000119772 | ENST00000264709 |
| chr2 | 25235703 | 25235828 | DNMT3A_exon_22 | − | DNMT3A | ENSG00000119772 | ENST00000264709 |
| chr2 | 25236932 | 25237008 | DNMT3A_exon_21 | − | DNMT3A | ENSG00000119772 | ENST00000264709 |
| chr2 | 25239126 | 25239218 | DNMT3A_exon_20 | − | DNMT3A | ENSG00000119772 | ENST00000264709 |
| chr2 | 25240298 | 25240453 | DNMT3A_exon_19 | − | DNMT3A | ENSG00000119772 | ENST00000264709 |
| chr2 | 25240636 | 25240733 | DNMT3A_exon_18 | − | DNMT3A | ENSG00000119772 | ENST00000264709 |
| chr2 | 25241558 | 25241710 | DNMT3A_exon_17 | − | DNMT3A | ENSG00000119772 | ENST00000264709 |
| chr2 | 25243894 | 25243985 | DNMT3A_exon_16 | − | DNMT3A | ENSG00000119772 | ENST00000264709 |
| chr2 | 25244151 | 25244341 | DNMT3A_exon_15 | − | DNMT3A | ENSG00000119772 | ENST00000264709 |
| chr2 | 25244536 | 25244655 | DNMT3A_exon_14 | − | DNMT3A | ENSG00000119772 | ENST00000264709 |
| chr2 | 25245249 | 25245335 | DNMT3A_exon_13 | − | DNMT3A | ENSG00000119772 | ENST00000264709 |
| chr2 | 25246016 | 25246067 | DNMT3A_exon_12 | − | DNMT3A | ENSG00000119772 | ENST00000264709 |
| chr2 | 25246156 | 25246312 | DNMT3A_exon_11 | − | DNMT3A | ENSG00000119772 | ENST00000264709 |
| chr2 | 25246616 | 25246779 | DNMT3A_exon_10 | − | DNMT3A | ENSG00000119772 | ENST00000264709 |
| chr2 | 25247047 | 25247161 | DNMT3A_exon_9 | − | DNMT3A | ENSG00000119772 | ENST00000264709 |
| chr2 | 25247587 | 25247752 | DNMT3A_exon_8 | − | DNMT3A | ENSG00000119772 | ENST00000264709 |
| chr2 | 25248033 | 25248255 | DNMT3A_exon_7 | − | DNMT3A | ENSG00000119772 | ENST00000264709 |
| chr2 | 25274937 | 25275090 | DNMT3A_exon_6 | − | DNMT3A | ENSG00000119772 | ENST00000264709 |
| chr2 | 25275496 | 25275546 | DNMT3A_exon_5 | − | DNMT3A | ENSG00000119772 | ENST00000264709 |
| chr2 | 25282437 | 25282714 | DNMT3A_exon_4 | − | DNMT3A | ENSG00000119772 | ENST00000264709 |
| chr2 | 25300135 | 25300246 | DNMT3A_exon_3 | − | DNMT3A | ENSG00000119772 | ENST00000264709 |
| chr2 | 25313909 | 25313987 | DNMT3A_exon_2 | − | DNMT3A | ENSG00000119772 | ENST00000264709 |
| chr20 | 32358772 | 32358835 | ASXL1_exon_1 | + | ASXL1 | ENSG00000171456 | ENST00000375687 |
| chr20 | 32366380 | 32366469 | ASXL1_exon_2 | + | ASXL1 | ENSG00000171456 | ENST00000375687 |
| chr20 | 32367723 | 32367732 | ASXL1_exon_3 | + | ASXL1 | ENSG00000171456 | ENST00000375687 |
| chr20 | 32369011 | 32369126 | ASXL1_exon_4 | + | ASXL1 | ENSG00000171456 | ENST00000375687 |
| chr20 | 32428124 | 32428251 | ASXL1_exon_5 | + | ASXL1 | ENSG00000171456 | ENST00000375687 |
| chr20 | 32428321 | 32428425 | ASXL1_exon_6 | + | ASXL1 | ENSG00000171456 | ENST00000375687 |
| chr20 | 32429334 | 32429434 | ASXL1_exon_7 | + | ASXL1 | ENSG00000171456 | ENST00000375687 |
| chr20 | 32429897 | 32430056 | ASXL1_exon_8 | + | ASXL1 | ENSG00000171456 | ENST00000375687 |
| chr20 | 32431317 | 32431487 | ASXL1_exon_9 | + | ASXL1 | ENSG00000171456 | ENST00000375687 |
| chr20 | 32431579 | 32431682 | ASXL1_exon_10 | + | ASXL1 | ENSG00000171456 | ENST00000375687 |
| chr20 | 32432876 | 32432988 | ASXL1_exon_11 | + | ASXL1 | ENSG00000171456 | ENST00000375687 |
| chr20 | 32433280 | 32433920 | ASXL1_exon_12 | + | ASXL1 | ENSG00000171456 | ENST00000375687 |
| chr20 | 32434428 | 32437338 | ASXL1_exon_13 | + | ASXL1 | ENSG00000171456 | ENST00000375687 |
| chr21 | 34792134 | 34792613 | RUNX1_exon_8 | − | RUNX1 | ENSG00000159216 | ENST00000300305 |
| chr21 | 34799297 | 34799465 | RUNX1_exon_7 | − | RUNX1 | ENSG00000159216 | ENST00000300305 |
| chr21 | 34834406 | 34834604 | RUNX1_exon_6 | − | RUNX1 | ENSG00000159216 | ENST00000300305 |
| chr21 | 34859470 | 34859581 | RUNX1_exon_5 | − | RUNX1 | ENSG00000159216 | ENST00000300305 |
| chr21 | 34880553 | 34880716 | RUNX1_exon_4 | − | RUNX1 | ENSG00000159216 | ENST00000300305 |
| chr21 | 34886839 | 34887099 | RUNX1_exon_3 | − | RUNX1 | ENSG00000159216 | ENST00000300305 |
| chr21 | 34892921 | 34892966 | RUNX1_exon_2 | − | RUNX1 | ENSG00000159216 | ENST00000300305 |
| chr21 | 35048838 | 35048902 | RUNX1_exon_1 | − | RUNX1 | ENSG00000159216 | ENST00000300305 |
| chr21 | 43094652 | 43094791 | U2AF1_exon_6 | − | U2AF1 | ENSG00000160201 | ENST00000291552 |
| chr21 | 43104312 | 43104405 | U2AF1_exon_2 | − | U2AF1 | ENSG00000160201 | ENST00000291552 |
| chr3 | 128481018 | 128481321 | GATA2_exon_6 | − | GATA2 | ENSG00000179348 | ENST00000341105 |
| chr3 | 128481815 | 128481947 | GATA2_exon_5 | − | GATA2 | ENSG00000179348 | ENST00000341105 |
| chr3 | 128483856 | 128484008 | GATA2_exon_4 | − | GATA2 | ENSG00000179348 | ENST00000341105 |
| chr3 | 128485723 | 128486371 | GATA2_exon_3 | − | GATA2 | ENSG00000179348 | ENST00000341105 |
| chr3 | 128486799 | 128487034 | GATA2_exon_2 | − | GATA2 | ENSG00000179348 | ENST00000341105 |
| chr4 | 105233939 | 105237354 | TET2_exon_3 | + | TET2 | ENSG00000168769 | ENST00000540549 |
| chr4 | 105241335 | 105241432 | TET2_exon_4 | + | TET2 | ENSG00000168769 | ENST00000540549 |
| chr4 | 105242830 | 105242930 | TET2_exon_5 | + | TET2 | ENSG00000168769 | ENST00000540549 |
| chr4 | 105243566 | 105243781 | TET2_exon_6 | + | TET2 | ENSG00000168769 | ENST00000540549 |
| chr4 | 105259615 | 105259772 | TET2_exon_7 | + | TET2 | ENSG00000168769 | ENST00000540549 |
| chr4 | 105261755 | 105261851 | TET2_exon_8 | + | TET2 | ENSG00000168769 | ENST00000540549 |
| chr4 | 105269606 | 105269750 | TET2_exon_9 | + | TET2 | ENSG00000168769 | ENST00000540549 |
| chr4 | 105272560 | 105272921 | TET2_exon_10 | + | TET2 | ENSG00000168769 | ENST00000540549 |
| chr4 | 105275044 | 105276519 | TET2_exon_11 | + | TET2 | ENSG00000168769 | ENST00000540549 |
| chr4 | 54695509 | 54695784 | KIT_exon_2 | + | KIT | ENSG00000157404 | ENST00000288135 |
| chr4 | 54723581 | 54723701 | KIT_exon_8 | + | KIT | ENSG00000157404 | ENST00000288135 |
| chr4 | 54725854 | 54726053 | KIT_exon_9 | + | KIT | ENSG00000157404 | ENST00000288135 |
| chr4 | 54727215 | 54727327 | KIT_exon_10 | + | KIT | ENSG00000157404 | ENST00000288135 |
| chr4 | 54727413 | 54727545 | KIT_exon_11 | + | KIT | ENSG00000157404 | ENST00000288135 |
| chr4 | 54728008 | 54728124 | KIT_exon_13 | + | KIT | ENSG00000157404 | ENST00000288135 |
| chr4 | 54733067 | 54733195 | KIT_exon_17 | + | KIT | ENSG00000157404 | ENST00000288135 |
| chr5 | 171410524 | 171410565 | NPM1_exon_11 | + | NPM1 | ENSG00000181163 | ENST00000296930 |
| chr7 | 101816027 | 101816096 | CUX1_exon_1 | + | CUX1 | ENSG00000257923 | ENST00000360264 |
| chr7 | 101916111 | 101916228 | CUX1_exon_2 | + | CUX1 | ENSG00000257923 | ENST00000360264 |
| chr7 | 102028094 | 102028148 | CUX1_exon_3 | + | CUX1 | ENSG00000257923 | ENST00000360264 |
| chr7 | 102070335 | 102070420 | CUX1_exon_4 | + | CUX1 | ENSG00000257923 | ENST00000360264 |
| chr7 | 102097360 | 102097504 | CUX1_exon_5 | + | CUX1 | ENSG00000257923 | ENST00000360264 |
| chr7 | 102104332 | 102104462 | CUX1_exon_6 | + | CUX1 | ENSG00000257923 | ENST00000360264 |
| chr7 | 102111694 | 102111777 | CUX1_exon_7 | + | CUX1 | ENSG00000257923 | ENST00000360264 |
| chr7 | 102115203 | 102115276 | CUX1_exon_8 | + | CUX1 | ENSG00000257923 | ENST00000360264 |
| chr7 | 102158556 | 102158611 | CUX1_exon_9 | + | CUX1 | ENSG00000257923 | ENST00000360264 |
| chr7 | 102170442 | 102170553 | CUX1_exon_10 | + | CUX1 | ENSG00000257923 | ENST00000360264 |
| chr7 | 102178465 | 102178660 | CUX1_exon_11 | + | CUX1 | ENSG00000257923 | ENST00000360264 |
| chr7 | 102189809 | 102189874 | CUX1_exon_12 | + | CUX1 | ENSG00000257923 | ENST00000360264 |
| chr7 | 102193838 | 102193893 | CUX1_exon_13 | + | CUX1 | ENSG00000257923 | ENST00000360264 |
| chr7 | 102195503 | 102195606 | CUX1_exon_14 | + | CUX1 | ENSG00000257923 | ENST00000360264 |
| chr7 | 102196630 | 102197308 | CUX1_exon_15 | + | CUX1 | ENSG00000257923 | ENST00000360264 |
| chr7 | 102198798 | 102198870 | CUX1_exon_16 | + | CUX1 | ENSG00000257923 | ENST00000360264 |
| chr7 | 102200067 | 102200175 | CUX1_exon_17 | + | CUX1 | ENSG00000257923 | ENST00000360264 |
| chr7 | 102201356 | 102202207 | CUX1_exon_18 | + | CUX1 | ENSG00000257923 | ENST00000360264 |
| chr7 | 102204387 | 102204559 | CUX1_exon_19 | + | CUX1 | ENSG00000257923 | ENST00000360264 |
| chr7 | 102205110 | 102205173 | CUX1_exon_20 | + | CUX1 | ENSG00000257923 | ENST00000360264 |
| chr7 | 102227363 | 102227672 | CUX1_exon_21 | + | CUX1 | ENSG00000257923 | ENST00000360264 |
| chr7 | 102234048 | 102234243 | CUX1_exon_22 | + | CUX1 | ENSG00000257923 | ENST00000360264 |
| chr7 | 102239316 | 102239587 | CUX1_exon_23 | + | CUX1 | ENSG00000257923 | ENST00000360264 |
| chr7 | 102248408 | 102249042 | CUX1_exon_24 | + | CUX1 | ENSG00000257923 | ENST00000360264 |
| chr7 | 140753272 | 140753396 | BRAF_exon_15 | − | BRAF | ENSG00000157764 | ENST00000288602 |
| chr7 | 148807645 | 148807709 | EZH2_exon_20 | − | EZH2 | ENSG00000106462 | ENST00000320356 |
| chr7 | 148809067 | 148809158 | EZH2_exon_19 | − | EZH2 | ENSG00000106462 | ENST00000320356 |
| chr7 | 148809306 | 148809393 | EZH2_exon_18 | − | EZH2 | ENSG00000106462 | ENST00000320356 |
| chr7 | 148810329 | 148810417 | EZH2_exon_17 | − | EZH2 | ENSG00000106462 | ENST00000320356 |
| chr7 | 148811621 | 148811723 | EZH2_exon_16 | − | EZH2 | ENSG00000106462 | ENST00000320356 |
| chr7 | 148813955 | 148814140 | EZH2_exon_15 | − | EZH2 | ENSG00000106462 | ENST00000320356 |
| chr7 | 148814910 | 148815042 | EZH2_exon_14 | − | EZH2 | ENSG00000106462 | ENST00000320356 |
| chr7 | 148815502 | 148815549 | EZH2_exon_13 | − | EZH2 | ENSG00000106462 | ENST00000320356 |
| chr7 | 148816680 | 148816781 | EZH2_exon_12 | − | EZH2 | ENSG00000106462 | ENST00000320356 |
| chr7 | 148817218 | 148817394 | EZH2_exon_11 | − | EZH2 | ENSG00000106462 | ENST00000320356 |
| chr7 | 148817873 | 148818120 | EZH2_exon_10 | − | EZH2 | ENSG00000106462 | ENST00000320356 |
| chr7 | 148819592 | 148819690 | EZH2_exon_9 | − | EZH2 | ENSG00000106462 | ENST00000320356 |
| chr7 | 148826450 | 148826635 | EZH2_exon_8 | − | EZH2 | ENSG00000106462 | ENST00000320356 |
| chr7 | 148827160 | 148827269 | EZH2_exon_7 | − | EZH2 | ENSG00000106462 | ENST00000320356 |
| chr7 | 148828736 | 148828883 | EZH2_exon_6 | − | EZH2 | ENSG00000106462 | ENST00000320356 |
| chr7 | 148829724 | 148829851 | EZH2_exon_5 | − | EZH2 | ENSG00000106462 | ENST00000320356 |
| chr7 | 148832630 | 148832753 | EZH2_exon_4 | − | EZH2 | ENSG00000106462 | ENST00000320356 |
| chr7 | 148846466 | 148846601 | EZH2_exon_3 | − | EZH2 | ENSG00000106462 | ENST00000320356 |
| chr7 | 148847178 | 148847301 | EZH2_exon_2 | − | EZH2 | ENSG00000106462 | ENST00000320356 |
| chr8 | 116847499 | 116847694 | RAD21_exon_14 | − | RAD21 | ENSG00000164754 | ENST00000297338 |
| chr8 | 116848942 | 116849032 | RAD21_exon_13 | − | RAD21 | ENSG00000164754 | ENST00000297338 |
| chr8 | 116850614 | 116850770 | RAD21_exon_12 | − | RAD21 | ENSG00000164754 | ENST00000297338 |
| chr8 | 116851944 | 116852099 | RAD21_exon_11 | − | RAD21 | ENSG00000164754 | ENST00000297338 |
| chr8 | 116852545 | 116852711 | RAD21_exon_10 | − | RAD21 | ENSG00000164754 | ENST00000297338 |
| chr8 | 116854241 | 116854471 | RAD21_exon_9 | − | RAD21 | ENSG00000164754 | ENST00000297338 |
| chr8 | 116856162 | 116856291 | RAD21_exon_8 | − | RAD21 | ENSG00000164754 | ENST00000297338 |
| chr8 | 116856642 | 116856774 | RAD21_exon_7 | − | RAD21 | ENSG00000164754 | ENST00000297338 |
| chr8 | 116857263 | 116857476 | RAD21_exon_6 | − | RAD21 | ENSG00000164754 | ENST00000297338 |
| chr8 | 116858348 | 116858461 | RAD21_exon_5 | − | RAD21 | ENSG00000164754 | ENST00000297338 |
| chr8 | 116861837 | 116861943 | RAD21_exon_4 | − | RAD21 | ENSG00000164754 | ENST00000297338 |
| chr8 | 116863126 | 116863262 | RAD21_exon_3 | − | RAD21 | ENSG00000164754 | ENST00000297338 |
| chr8 | 116866582 | 116866732 | RAD21_exon_2 | − | RAD21 | ENSG00000164754 | ENST00000297338 |
| chr9 | 5069922 | 5070055 | JAK2_exon_12 | + | JAK2 | ENSG00000096968 | ENST00000381652 |
| chr9 | 5073695 | 5073788 | JAK2_exon_14 | + | JAK2 | ENSG00000096968 | ENST00000381652 |
| chrX | 124022624 | 124022674 | STAG2_exon_3 | + | STAG2 | ENSG00000101972 | ENST00000218089 |
| chrX | 124025836 | 124025921 | STAG2_exon_4 | + | STAG2 | ENSG00000101972 | ENST00000218089 |
| chrX | 124030957 | 124031128 | STAG2_exon_5 | + | STAG2 | ENSG00000101972 | ENST00000218089 |
| chrX | 124037523 | 124037626 | STAG2_exon_6 | + | STAG2 | ENSG00000101972 | ENST00000218089 |
| chrX | 124042565 | 124042648 | STAG2_exon_7 | + | STAG2 | ENSG00000101972 | ENST00000218089 |
| chrX | 124045160 | 124045371 | STAG2_exon_8 | + | STAG2 | ENSG00000101972 | ENST00000218089 |
| chrX | 124047350 | 124047508 | STAG2_exon_9 | + | STAG2 | ENSG00000101972 | ENST00000218089 |
| chrX | 124049001 | 124049081 | STAG2_exon_10 | + | STAG2 | ENSG00000101972 | ENST00000218089 |
| chrX | 124050182 | 124050312 | STAG2_exon_11 | + | STAG2 | ENSG00000101972 | ENST00000218089 |
| chrX | 124051117 | 124051222 | STAG2_exon_12 | + | STAG2 | ENSG00000101972 | ENST00000218089 |
| chrX | 124051311 | 124051397 | STAG2_exon_13 | + | STAG2 | ENSG00000101972 | ENST00000218089 |
| chrX | 124056124 | 124056238 | STAG2_exon_14 | + | STAG2 | ENSG00000101972 | ENST00000218089 |
| chrX | 124057862 | 124057980 | STAG2_exon_15 | + | STAG2 | ENSG00000101972 | ENST00000218089 |
| chrX | 124061220 | 124061344 | STAG2_exon_16 | + | STAG2 | ENSG00000101972 | ENST00000218089 |
| chrX | 124061767 | 124061877 | STAG2_exon_17 | + | STAG2 | ENSG00000101972 | ENST00000218089 |
| chrX | 124062898 | 124062997 | STAG2_exon_18 | + | STAG2 | ENSG00000101972 | ENST00000218089 |
| chrX | 124063112 | 124063208 | STAG2_exon_19 | + | STAG2 | ENSG00000101972 | ENST00000218089 |
| chrX | 124063844 | 124064054 | STAG2_exon_20 | + | STAG2 | ENSG00000101972 | ENST00000218089 |
| chrX | 124065872 | 124065949 | STAG2_exon_21 | + | STAG2 | ENSG00000101972 | ENST00000218089 |
| chrX | 124066171 | 124066265 | STAG2_exon_22 | + | STAG2 | ENSG00000101972 | ENST00000218089 |
| chrX | 124066352 | 124066439 | STAG2_exon_23 | + | STAG2 | ENSG00000101972 | ENST00000218089 |
| chrX | 124068560 | 124068659 | STAG2_exon_24 | + | STAG2 | ENSG00000101972 | ENST00000218089 |
| chrX | 124071145 | 124071326 | STAG2_exon_25 | + | STAG2 | ENSG00000101972 | ENST00000218089 |
| chrX | 124076328 | 124076474 | STAG2_exon_26 | + | STAG2 | ENSG00000101972 | ENST00000218089 |
| chrX | 124077953 | 124078061 | STAG2_exon_27 | + | STAG2 | ENSG00000101972 | ENST00000218089 |
| chrX | 124081376 | 124081531 | STAG2_exon_28 | + | STAG2 | ENSG00000101972 | ENST00000218089 |
| chrX | 124083417 | 124083552 | STAG2_exon_29 | + | STAG2 | ENSG00000101972 | ENST00000218089 |
| chrX | 124086543 | 124086773 | STAG2_exon_30 | + | STAG2 | ENSG00000101972 | ENST00000218089 |
| chrX | 124090571 | 124090767 | STAG2_exon_31 | + | STAG2 | ENSG00000101972 | ENST00000218089 |
| chrX | 124090850 | 124090967 | STAG2_exon_32 | + | STAG2 | ENSG00000101972 | ENST00000218089 |
| chrX | 124094014 | 124094147 | STAG2_exon_33 | + | STAG2 | ENSG00000101972 | ENST00000218089 |
| chrX | 124095368 | 124095452 | STAG2_exon_34 | + | STAG2 | ENSG00000101972 | ENST00000218089 |
| chrX | 124100570 | 124100597 | STAG2_exon_35 | + | STAG2 | ENSG00000101972 | ENST00000218089 |
| chrX | 130005228 | 130005320 | BCORL1_exon_1 | + | BCORL1 | ENSG00000085185 | ENST00000540052 |
| chrX | 130012574 | 130012671 | BCORL1_exon_2 | + | BCORL1 | ENSG00000085185 | ENST00000540052 |
| chrX | 130012946 | 130016216 | BCORL1_exon_3 | + | BCORL1 | ENSG00000085185 | ENST00000540052 |
| chrX | 130020981 | 130021153 | BCORL1_exon_4 | + | BCORL1 | ENSG00000085185 | ENST00000540052 |
| chrX | 130022893 | 130022980 | BCORL1_exon_5 | + | BCORL1 | ENSG00000085185 | ENST00000540052 |
| chrX | 130024986 | 130025382 | BCORL1_exon_6 | + | BCORL1 | ENSG00000085185 | ENST00000540052 |
| chrX | 130028631 | 130028864 | BCORL1_exon_7 | + | BCORL1 | ENSG00000085185 | ENST00000540052 |
| chrX | 130037363 | 130037536 | BCORL1_exon_8 | + | BCORL1 | ENSG00000085185 | ENST00000540052 |
| chrX | 130039133 | 130039285 | BCORL1_exon_9 | + | BCORL1 | ENSG00000085185 | ENST00000540052 |
| chrX | 130050713 | 130050797 | BCORL1_exon_10 | + | BCORL1 | ENSG00000085185 | ENST00000540052 |
| chrX | 130051856 | 130052019 | BCORL1_exon_11 | + | BCORL1 | ENSG00000085185 | ENST00000540052 |
| chrX | 130055850 | 130056136 | BCORL1_exon_12 | + | BCORL1 | ENSG00000085185 | ENST00000540052 |
| chrX | 134377614 | 134377758 | PHF6_exon_2 | + | PHF6 | ENSG00000156531 | ENST00000332070 |
| chrX | 134378001 | 134378109 | PHF6_exon_3 | + | PHF6 | ENSG00000156531 | ENST00000332070 |
| chrX | 134393497 | 134393637 | PHF6_exon_4 | + | PHF6 | ENSG00000156531 | ENST00000332070 |
| chrX | 134393905 | 134393955 | PHF6_exon_5 | + | PHF6 | ENSG00000156531 | ENST00000332070 |
| chrX | 134413487 | 134413660 | PHF6_exon_6 | + | PHF6 | ENSG00000156531 | ENST00000332070 |
| chrX | 134413819 | 134413969 | PHF6_exon_7 | + | PHF6 | ENSG00000156531 | ENST00000332070 |
| chrX | 134415012 | 134415123 | PHF6_exon_8 | + | PHF6 | ENSG00000156531 | ENST00000332070 |
| chrX | 134417165 | 134417305 | PHF6_exon_9 | + | PHF6 | ENSG00000156531 | ENST00000332070 |
| chrX | 134425197 | 134425330 | PHF6_exon_10 | + | PHF6 | ENSG00000156531 | ENST00000332070 |
| chrX | 15321505 | 15321775 | PIGA_exon_6 | − | PIGA | ENSG00000165195 | ENST00000333590 |
| chrX | 15324661 | 15324874 | PIGA_exon_5 | − | PIGA | ENSG00000165195 | ENST00000333590 |
| chrX | 15325016 | 15325155 | PIGA_exon_4 | − | PIGA | ENSG00000165195 | ENST00000333590 |
| chrX | 15325910 | 15326049 | PIGA_exon_3 | − | PIGA | ENSG00000165195 | ENST00000333590 |
| chrX | 15331212 | 15331933 | PIGA_exon_2 | − | PIGA | ENSG00000165195 | ENST00000333590 |
| chrX | 15790492 | 15790539 | ZRSR2_exon_1 | + | ZRSR2 | ENSG00000169249 | ENST00000307771 |
| chrX | 15790930 | 15791016 | ZRSR2_exon_2 | + | ZRSR2 | ENSG00000169249 | ENST00000307771 |
| chrX | 15799868 | 15799956 | ZRSR2_exon_3 | + | ZRSR2 | ENSG00000169249 | ENST00000307771 |
| chrX | 15803684 | 15803799 | ZRSR2_exon_4 | + | ZRSR2 | ENSG00000169249 | ENST00000307771 |
| chrX | 15804107 | 15804200 | ZRSR2_exon_5 | + | ZRSR2 | ENSG00000169249 | ENST00000307771 |
| chrX | 15808229 | 15808274 | ZRSR2_exon_6 | + | ZRSR2 | ENSG00000169249 | ENST00000307771 |
| chrX | 15809196 | 15809321 | ZRSR2_exon_7 | + | ZRSR2 | ENSG00000169249 | ENST00000307771 |
| chrX | 15815673 | 15815893 | ZRSR2_exon_8 | + | ZRSR2 | ENSG00000169249 | ENST00000307771 |
| chrX | 15818583 | 15818645 | ZRSR2_exon_9 | + | ZRSR2 | ENSG00000169249 | ENST00000307771 |
| chrX | 15820203 | 15820319 | ZRSR2_exon_10 | + | ZRSR2 | ENSG00000169249 | ENST00000307771 |
| chrX | 15822727 | 15823242 | ZRSR2_exon_11 | + | ZRSR2 | ENSG00000169249 | ENST00000307771 |
| chrX | 40052108 | 40052403 | BCOR_exon_15 | − | BCOR | ENSG00000183337 | ENST00000378444 |
| chrX | 40053882 | 40054045 | BCOR_exon_14 | − | BCOR | ENSG00000183337 | ENST00000378444 |
| chrX | 40054252 | 40054336 | BCOR_exon_13 | − | BCOR | ENSG00000183337 | ENST00000378444 |
| chrX | 40055364 | 40055516 | BCOR_exon_12 | − | BCOR | ENSG00000183337 | ENST00000378444 |
| chrX | 40057151 | 40057324 | BCOR_exon_11 | − | BCOR | ENSG00000183337 | ENST00000378444 |
| chrX | 40062135 | 40062396 | BCOR_exon_10 | − | BCOR | ENSG00000183337 | ENST00000378444 |
| chrX | 40062742 | 40063074 | BCOR_exon_9 | − | BCOR | ENSG00000183337 | ENST00000378444 |
| chrX | 40063604 | 40063955 | BCOR_exon_8 | − | BCOR | ENSG00000183337 | ENST00000378444 |
| chrX | 40064332 | 40064602 | BCOR_exon_7 | − | BCOR | ENSG00000183337 | ENST00000378444 |
| chrX | 40070969 | 40071162 | BCOR_exon_6 | − | BCOR | ENSG00000183337 | ENST00000378444 |
| chrX | 40071633 | 40071693 | BCOR_exon_5 | − | BCOR | ENSG00000183337 | ENST00000378444 |
| chrX | 40072345 | 40075183 | BCOR_exon_4 | − | BCOR | ENSG00000183337 | ENST00000378444 |
| chrX | 40076450 | 40076535 | BCOR_exon_3 | − | BCOR | ENSG00000183337 | ENST00000378444 |
| chrX | 40077840 | 40077932 | BCOR_exon_2 | − | BCOR | ENSG00000183337 | ENST00000378444 |
| chrX | 53380102 | 53380189 | SMC1A_exon_25 | − | SMC1A | ENSG00000072501 | ENST00000322213 |
| chrX | 53380616 | 53380733 | SMC1A_exon_24 | − | SMC1A | ENSG00000072501 | ENST00000322213 |
| chrX | 53381014 | 53381090 | SMC1A_exon_23 | − | SMC1A | ENSG00000072501 | ENST00000322213 |
| chrX | 53382228 | 53382386 | SMC1A_exon_22 | − | SMC1A | ENSG00000072501 | ENST00000322213 |
| chrX | 53382502 | 53382663 | SMC1A_exon_21 | − | SMC1A | ENSG00000072501 | ENST00000322213 |
| chrX | 53383093 | 53383256 | SMC1A_exon_20 | − | SMC1A | ENSG00000072501 | ENST00000322213 |
| chrX | 53394774 | 53394891 | SMC1A_exon_19 | − | SMC1A | ENSG00000072501 | ENST00000322213 |
| chrX | 53396223 | 53396383 | SMC1A_exon_18 | − | SMC1A | ENSG00000072501 | ENST00000322213 |
| chrX | 53396468 | 53396620 | SMC1A_exon_17 | − | SMC1A | ENSG00000072501 | ENST00000322213 |
| chrX | 53399585 | 53399733 | SMC1A_exon_16 | − | SMC1A | ENSG00000072501 | ENST00000322213 |
| chrX | 53403562 | 53403675 | SMC1A_exon_15 | − | SMC1A | ENSG00000072501 | ENST00000322213 |
| chrX | 53403773 | 53403896 | SMC1A_exon_14 | − | SMC1A | ENSG00000072501 | ENST00000322213 |
| chrX | 53405008 | 53405152 | SMC1A_exon_13 | − | SMC1A | ENSG00000072501 | ENST00000322213 |
| chrX | 53405241 | 53405394 | SMC1A_exon_12 | − | SMC1A | ENSG00000072501 | ENST00000322213 |
| chrX | 53405489 | 53405675 | SMC1A_exon_11 | − | SMC1A | ENSG00000072501 | ENST00000322213 |
| chrX | 53405767 | 53405959 | SMC1A_exon_10 | − | SMC1A | ENSG00000072501 | ENST00000322213 |
| chrX | 53409058 | 53409272 | SMC1A_exon_9 | − | SMC1A | ENSG00000072501 | ENST00000322213 |
| chrX | 53409417 | 53409506 | SMC1A_exon_8 | − | SMC1A | ENSG00000072501 | ENST00000322213 |
| chrX | 53411757 | 53411904 | SMC1A_exon_7 | − | SMC1A | ENSG00000072501 | ENST00000322213 |
| chrX | 53411991 | 53412256 | SMC1A_exon_6 | − | SMC1A | ENSG00000072501 | ENST00000322213 |
| chrX | 53412896 | 53413141 | SMC1A_exon_5 | − | SMC1A | ENSG00000072501 | ENST00000322213 |
| chrX | 53413228 | 53413438 | SMC1A_exon_4 | − | SMC1A | ENSG00000072501 | ENST00000322213 |
| chrX | 53414754 | 53414873 | SMC1A_exon_3 | − | SMC1A | ENSG00000072501 | ENST00000322213 |
| chrX | 53414977 | 53415172 | SMC1A_exon_2 | − | SMC1A | ENSG00000072501 | ENST00000322213 |
| chrX | 53422488 | 53422603 | SMC1A_exon_1 | − | SMC1A | ENSG00000072501 | ENST00000322213 |

Confirmatory Studies

We used FISH, PCR, and chromosomal microarray analyses, with or without existing RNA-sequencing data, to confirm findings on whole-genome sequencing that had not been detected by cytogenetic analysis. We used standard protocols to perform chromosomal microarray analysis in the Washington University Cytogenetics Core. In the PCR-confirmation analyses, we used primers designed to detect structural variant breakpoints. The methods that were used in RNA sequencing for structural variants in selected samples have been reported previously.

WGS results were compared to conventional cytogenetics and FISH to determine the sensitivity and positive predictive value for detecting recurrent SVs and CNAs. These comparisons used the following approaches:
SVs: Cases with successful cytogenetics (at least 3 metaphases analyzed, N=235) were used to evaluate SV performance. SVs identified by WGS were manually compared to ISCN karyotypes obtained from clinical testing to identify true positives. Breakpoints were required to occur within 1 chromosome band. New SVs that were not reported by conventional cytogenetics were subject to confirmation using either FISH, PCR for SV breakpoints, or analysis of existing RNA-seq data for fusion transcripts (see below).
CNAs: CNAs from WGS were compared to ISCN karyotypes using 143 cases with conclusive cytogenetic results (i.e., 20 metaphases) and no ambiguous findings, such as composite karyotypes, marker chromosomes, or additional unidentifiable material, as these preclude definitive comparisons. ISCN cytogenetic karyotypes were transformed into a matrix of gains and losses for each chromosome band using published software, which were then converted with a custom PERL script to BEDPE format using band coordinates based on the GRCh38 human reference. The bedtools program was then used to compare CNAs between WGS and cytogenetics using at least 1 bp of overlap to identify concordant events. New CNAs were subject to confirmation using either FISH or chromosomal microarrays (CMA).

Every effort was made to confirm all novel findings, although priority was given to findings in the prospective cohort and for risk-defining events. Specific confirmation procedures are described below.

FISH

WGS findings not present in the karyotype or confirmed by diagnostic FISH results were confirmed using FISH studies where possible. FISH was the primary means of confirmation for new SVs and CNAs when appropriate probes were available and clinical specimens were available for testing. All FISH studies were performed using validated probes and standard clinical procedures using 200 cells and were reviewed by board certified cytogeneticists. The presence of an abnormal result in the specified study was considered as support for the genomic event identified by WGS. For example, we considered an abnormal result for the KMT2A dual color/dual fusion FISH assay as confirmation of an SV involving KTM2A in the WGS data.

PCR

Selected SVs that could not be confirmed via FISH because of insufficient or inadequate samples were confirmed via PCR from DNA using primers spanning the SV breakends identified by Manta when FISH studies could not be performed due to limited material and/or lack of appropriate FISH probes. PCR primers were designed from breakpoint-spanning sequence contigs generated from Manta and were used in standard PCR reactions with human genomic DNA. Amplified fragments were excised, purified, sequenced with Sanger sequencing, and analyzed with Blat to verify localization to the breakpoint region.

CMA

CNAs were confirmed via chromosomal microarray (CMA) for cases with available DNA but insufficient material or probe for FISH assays. CMAs were performed per standard methods using the CytoScan HD platform (ThermoFisher) with subsequent analysis in Chromosome Analysis Suite (ThermoFisher). Data were reviewed and interpreted by a board-certified cytogeneticist and molecular geneticist.

RNA-Seq

SVs in two cases with KMT2A rearrangements were confirmed using existing RNA-seq data that was published as part of the TCGA AML study (see Supplemental Table 1 in ref 18, which can be accessed here: https://api.gdc.cancer.gov/data/b9196563-a05d-40b8-80dc-640ec712eb06; samples 380949 and 410324). We note that clinical FISH using a KMT2A breakapart probe for these cases was also abnormal, and the identification of a fusion transcript via RNA-seq provided the definitive confirmation of the translocation partner.

### Risk Stratification

Conventional

T provide a basis of comparison for risk stratification results obtained using the disclosed WGS method, cytogenetics, FISH, and molecular results were used to assign patients to established genomic risk categories, which used the 2017 ELN guidelines for AML patients12 and the cytogenetic component of the IPSS-R scoring system for MDS patients, both without modification. Cytogenetic abnormalities were required to meet the abovementioned criteria to be considered clonal. For AML patients, risk group assignment was performed using cytogenetic results, FLT3 ITD mutation allele ratio from PCR (or presence/absence if the allelic ratio was not available), and the mutation status for CEBPA, NPM1, TP53, RUNX1, and ASXL1 from either clinical tumor/normal exome sequencing (N=12) or gene panel sequencing (using Myeloseq, N=71, or a commercial assay, N=1). Sequencing assays were not performed for 6 retrospective patients who were either assigned to a risk group using only NPM1 and FLT3 ITD mutation status (N=3), or they were assigned to intermediate risk (N=3). Patients with a normal karyotype and <20 metaphases were not assigned to a risk group with unless there was an unequivocal result from either FISH or targeted sequencing (e.g., a positive PML-RARA or del(5q) by FISH, or a TP53 mutation by targeted sequencing). IPSS-R risk groups do not involve gene mutations and are therefore performed using cytogenetics alone.

WGS

WGS results were used to assign patients to risk groups using the identical guidelines as above for both AML and MDS patients. For AML patients, risk assignment used CNAs, recurrent SVs, and gene mutations. FLT3 ITD mutation results from PCR were used instead of the WGS result (even though ITD alleles can be detected) because the PCR assay is an FDA-cleared companion diagnostic for the FLT3 targeted therapy midostaurin. For both AML and MDS patients, the clinically important classifications of normal karyotype and complex karyotype used only CNAs and recurrent SVs and not SVs reported as secondary findings. A normal karyotype was designated if no variants in either category were identified, and a complex karyotype was designated if at least 3 chromosomal abnormalities were identified, including recurrent SVs (not WHO category-defining events) or CNAs greater than 5 Mbp that were identified by copy number analysis and that involved separate chromosome arms. All but 3 of the patients with a complex karyotype could be assigned to this category based on CNAs alone, which indicates that copy number gains and losses are defining features of this phenotype.

### Statistics

Statistical Analysis

In the time-to-event survival analysis involving study patients with AML, we used death as the end point for the Kaplan-Meier analysis or Cox proportional hazards regression to test for equal survival across genetic risk groups. Censoring of patients in these analyses was random and occurred because of limited follow-up time. Survival analyses of patients with defined cytogenetic risk (N=71 nontransplanted patients; N=101 total patients) was pre-planned using patients within our cohort (i.e., they were not selected specifically for outcome analysis) and was performed by Kaplan-Meier analyses using the log-rank test for equal survival across the groups. Cox proportional-hazards regression was used to calculate hazard ratios and test for equal survival between the adverse risk group and either intermediate, favorable, or a combined intermediate/favorable ‘not adverse’ risk group. All log-rank tests performed in the paper were adjusted for multiple comparisons using the method of Benjamini and Hochberg (1995). Cox regression was adjusted for age (binned by decade), which was significantly associated with overall survival in the 71 non-transplanted patients with defined risk stratified by conventional risk groups (HR: 1.46, 95% CI 1.05-2.05) but not WGS-based risk groups (HR: 1.29, 95% CI 0.92-1.81). The log of the white blood cell count was also used as a covariate with ELN risk, but was not significant in any analysis (P>0.05 in all analyses) and therefore was not included in the model. The proportional hazards assumption was found to be tenable for all Cox models.

The same approaches were used for AML patients with undefined cytogenetic risk (N=27 nontransplanted patients; N=38 total patients). Prior to this pre-planned analysis, we performed a power calculation to estimate the sample size necessary to observe a difference in survival among ELN risk groups in this cohort. This used the Power and Sample Size task in SAS/Studio software along with the observed survival in the defined cytogenetic risk cohort above (N=71), which was largely consistent with published data on a mixture of older (60 and over) and younger (less than 60) patients. The power calculation used a median survival of 3600 days of survival for the favorable group and 346 for the adverse group, with a minimum follow-up interval of 279 days and a total number of days (accrual+follow-up) of 750 days. This demonstrated 80% power to detect a survival difference between favorable and adverse risk at a sample size of 12 (per group) using an alpha of 0.05. Additional exploratory analyses were performed but not presented, including log-rank tests for differences in survival among all three risk groups (rather than not adverse vs. adverse) and unadjusted Cox regression tests, which yielded similar results to those shown here. Survival statistics were obtained using SAS for Windows, Version 9.4. The survminer package in R was used for visualization.

### Results

### Streamlined Approach to Whole-Genome Sequencing

We developed a streamlined approach to whole-genome sequencing (ChromoSeq) that was designed to provide comprehensive genomic profiling of clinically relevant mutations in samples obtained from patients with AML or MDS, while minimizing the turnaround time and technical complexity (FIG. 5). In this approach, we used scalable methods of sample preparation that can be performed by a single technician in less than 8 hours with commercially available reagents, followed by standard high-throughput sequencing. Automated tumor-only variant analysis detected mutations in selected genes, copy-number alterations of more than 5 Mbp, and recurrent structural variants (Tables S1 and S2, above). We then summarized these findings in a concise clinical report (FIG. 9).

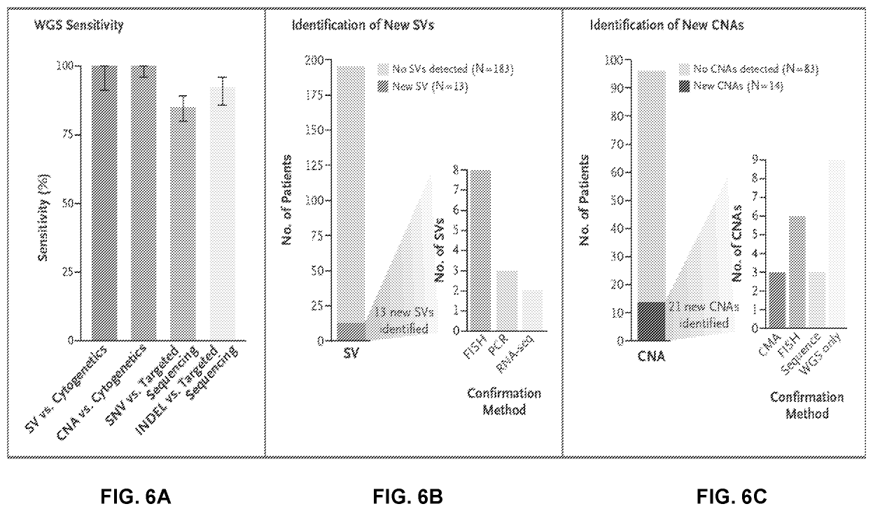

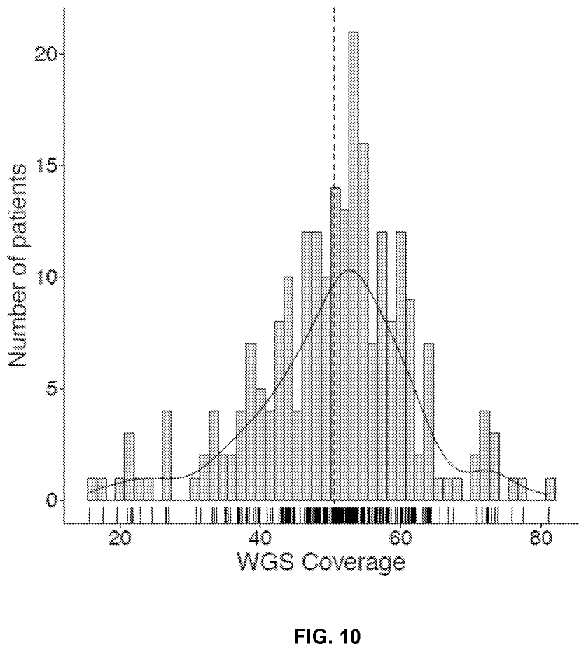

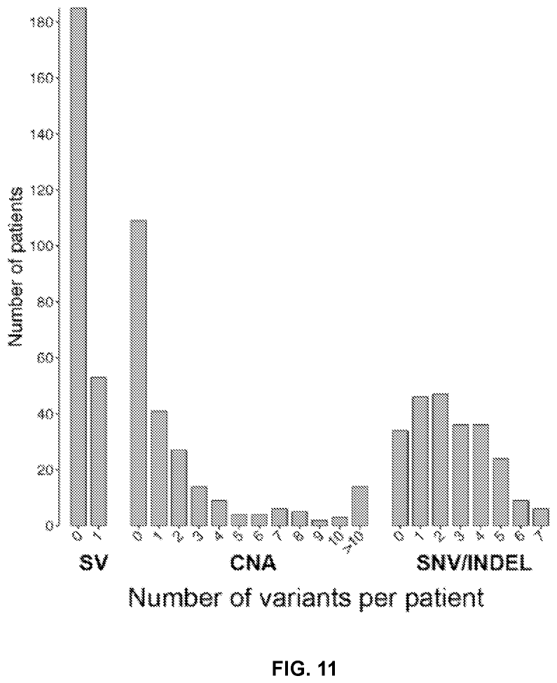

We performed a head-to-head comparison of this approach with conventional cytogenetic analysis and targeted sequencing using 235 samples obtained from patients with a known or suspected hematologic cancer who had undergone successful cytogenetic analysis. This sequencing analysis yielded a mean genome coverage of 50×; a mean of 5.1 clinically relevant mutations (range, 0 to 20) were detected per patient across all variant types (FIGS. 10 and 11). The sensitivity of whole-genome sequencing for recurrent translocations that had been reported on cytogenetic analysis was 100% (40 of 40 samples) (FIG. 6A).

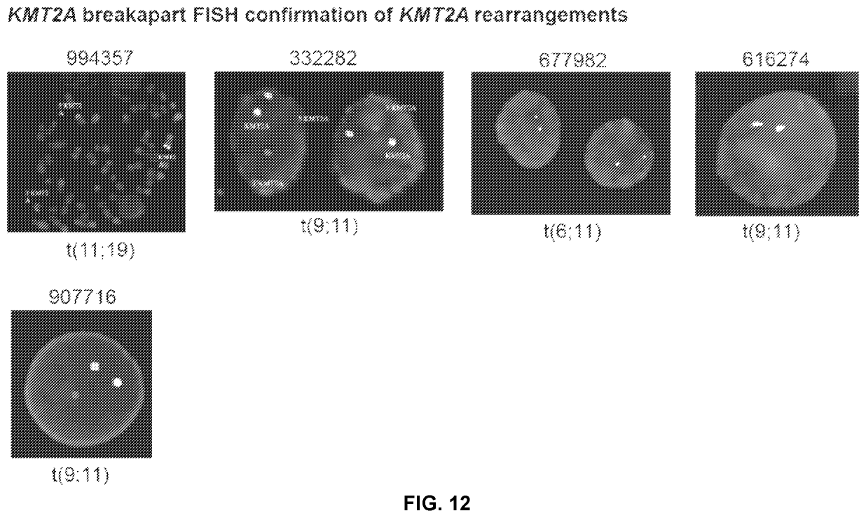

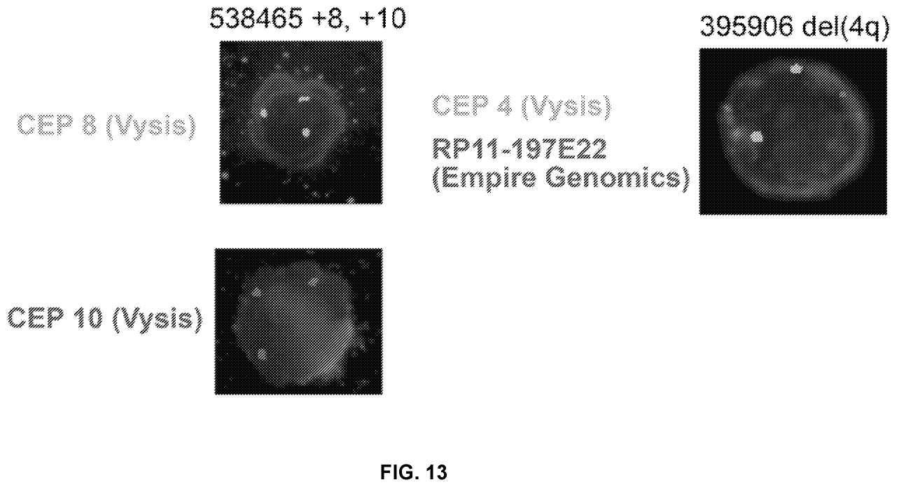

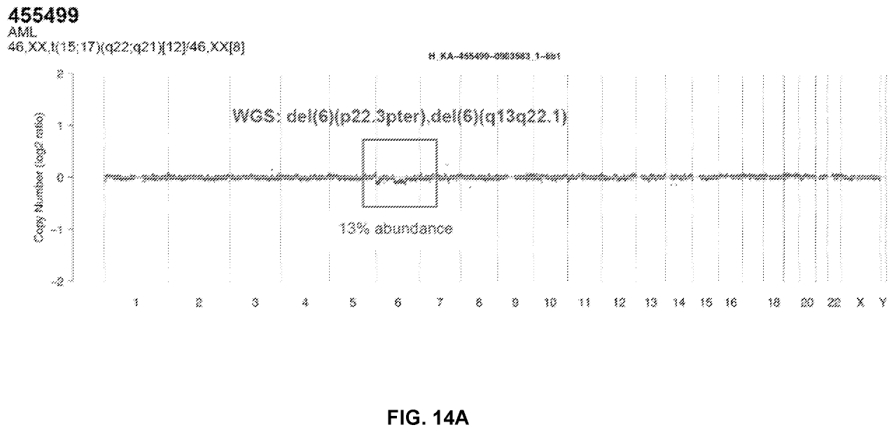

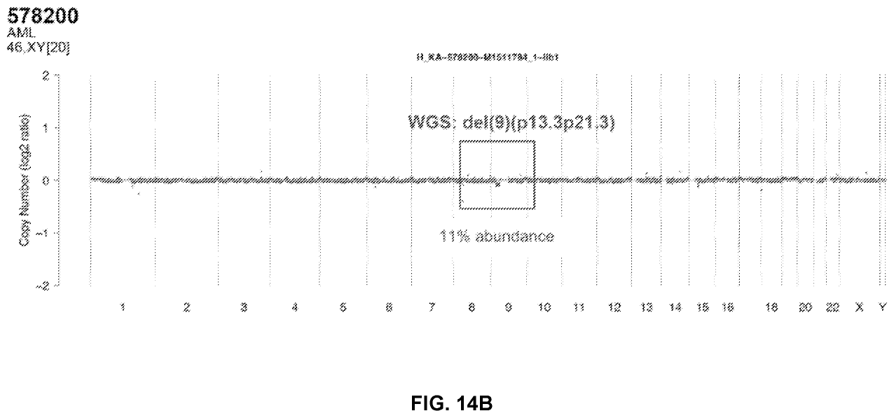

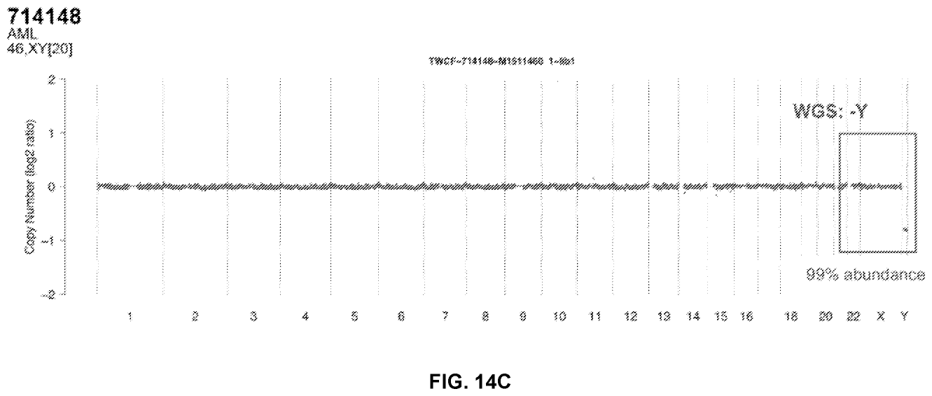

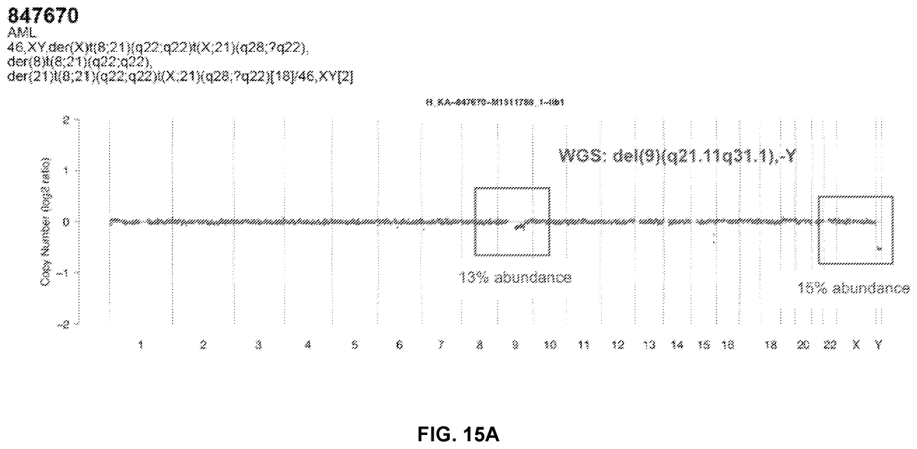

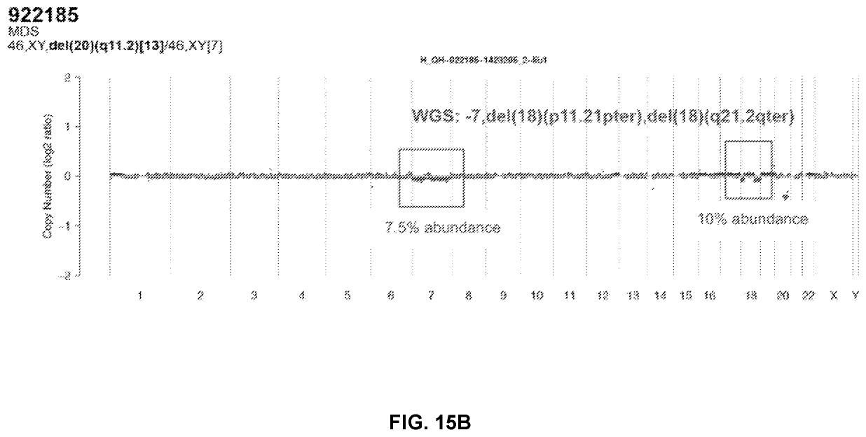

Whole-genome sequencing identified cytogenetically cryptic structural variants in 13 patients, including complex or cryptic chromosomal translocations involving the inv(16)(p13.1q22) fusion gene CBFB-MYH11 in 2 patients, the t(7;21)(p22;q22) fusion gene USP42-RUNX1 in 1 patient, and 10 rearrangements involving KMT2A, all of which were verified with the use of orthogonal methods (FIG. 6B and FIG. 12, Whole-genome sequencing detected 100% (91 of 91) of the clonal copy-number alterations that had been detected on cytogenetic analysis among the 143 patients in whom conclusive and unambiguous results had been identified by karyotyping (FIG. 6A). In addition, sequencing identified 21 new copy-number alterations in 14 of these patients, 12 of which were confirmed by other methods (FIG. 6C). The remaining 9 new copy-number alterations showed altered coverage patterns on whole-genome sequencing but could not be confirmed by orthogonal methods because of their small size, low abundance, or both (FIGS. 6C, 14A, 14B, and 14C). Whole-genome sequencing also provided definitive identification of copy-number alterations in an additional 13 patients with ambiguous or inconclusive results by cytogenetic analysis (Table S5). When we combined these results with the findings in 14 patients who had conclusive results by cytogenetic analysis and newly identified copy-number alterations, plus the findings in 13 patients who were identified as having new structural variants (see Table S4), we determined that 40 of 235 patients (17.0%) had results that had not been detected by conventional cytogenetic analysis.

TABLE S5
New CNAs Identified by WGS

| Chrom | Start | End | Size | Bands | Type | Diagnosis | WGS.CNAs | WGS.Recurrent.SVs |
|---|---|---|---|---|---|---|---|---|
| chr16 | 61500000 | 90000000 | 28500000 | q21qter | DEL | AML | del(3)(p11.2pter)[61.8%], +5[76.7%], | 0 |
| | | | | | | | del(5)(q11.2qter)[60.6%], −7[57.2%], +8[57.6%], +9[52.3%], | |
| | | | | | | | gain(15)(q11.2qter)[43.2%], del(16)(q21qter)[58.5%], | |
| | | | | | | | del(17)(p13.1pter)[53.0%], +18[43.5%], gain(22)(q11.21q12.3)[64.0%] | |
| chr17 | 500000 | 10500000 | 1.00E+07 | pterp13.1 | DEL | AML | del(3)(p11.2pter)[61.8%], +5[76.7%], | 0 |
| | | | | | | | del(5)(q11.2qter)[60.6%], −7[57.2%], +8[57.6%], +9[52.3%], | |
| | | | | | | | gain(15)(q11.2qter)[43.2%], del(16)(q21qter)[58.5%], | |
| | | | | | | | del(17)(p13.1pter)[53.0%], +18[43.5%], gain(22)(q11.21q12.3)[64.0%] | |
| chr18 | 500000 | 80000000 | 79500000 | pterqter | DUP | AML | del(3)(p11.2pter)[61.8%], +5[76.7%], | 0 |
| | | | | | | | del(5)(q11.2qter)[60.6%], −7[57.2%], +8[57.6%], +9[52.3%], | |
| | | | | | | | gain(15)(q11.2qter)[43.2%], del(16)(q21qter)[58.5%], | |
| | | | | | | | del(17)(p13.1pter)[53.0%], +18[43.5%], gain(22)(q11.21q12.3)[64.0%] | |
| chr22 | 17500000 | 36500000 | 1.90E+07 | q11.21q12.3 | DUP | AML | del(3)(p11.2pter)[61.8%], +5[76.7%], | 0 |
| | | | | | | | del(5)(q11.2qter)[60.6%], −7[57.2%], +8[57.6%], +9[52.3%], | |
| | | | | | | | gain(15)(q11.2qter)[43.2%], del(16)(q21qter)[58.5%], | |
| | | | | | | | del(17)(p13.1pter)[53.0%], +18[43.5%], gain(22)(q11.21q12.3)[64.0%] | |
| chr3 | 1000000 | 87500000 | 86500000 | pterp11.2 | DEL | AML | del(3)(p11.2pter)[61.8%], +5[76.7%], | 0 |
| | | | | | | | del(5)(q11.2qter)[60.6%], −7[57.2%], +8[57.6%], +9[52.3%], | |
| | | | | | | | gain(15)(q11.2qter)[43.2%], del(16)(q21qter)[58.5%], | |
| | | | | | | | del(17)(p13.1pter)[53.0%], +18[43.5%], gain(22)(q11.21q12.3)[64.0%] | |
| chr5 | 1000000 | 52000000 | 5.10E+07 | pterq11.2 | DUP | AML | del(3)(p11.2pter)[61.8%], +5[76.7%], | 0 |
| | | | | | | | del(5)(q11.2qter)[60.6%], −7[57.2%], +8[57.6%], +9[52.3%], | |
| | | | | | | | gain(15)(q11.2qter)[43.2%], del(16)(q21qter)[58.5%], | |
| | | | | | | | del(17)(p13.1pter)[53.0%], +18[43.5%], gain(22)(q11.21q12.3)[64.0%] | |
| chr5 | 52000000 | 181000000 | 1.29E+08 | q11.2qter | DEL | AML | del(3)(p11.2pter)[61.8%], +5[76.7%], | 0 |
| | | | | | | | del(5)(q11.2qter)[60.6%], −7[57.2%], +8[57.6%], +9[52.3%], | |
| | | | | | | | gain(15)(q11.2qter)[43.2%], del(16)(q21qter)[58.5%], | |
| | | | | | | | del(17)(p13.1pter)[53.0%], +18[43.5%], gain(22)(q11.21q12.3)[64.0%] | |
| chr7 | 500000 | 159000000 | 158500000 | pterqter | DEL | AML | del(3)(p11.2pter)[61.8%], +5[76.7%], | 0 |
| | | | | | | | del(5)(q11.2qter)[60.6%], −7[57.2%], +8[57.6%], +9[52.3%], | |
| | | | | | | | gain(15)(q11.2qter)[43.2%], del(16)(q21qter)[58.5%], | |
| | | | | | | | del(17)(p13.1pter)[53.0%], +18[43.5%], gain(22)(q11.21q12.3)[64.0%] | |
| chr8 | 500000 | 145000000 | 144500000 | pterqter | DUP | AML | del(3)(p11.2pter)[61.8%], +5[76.7%], | 0 |
| | | | | | | | del(5)(q11.2qter)[60.6%], −7[57.2%], +8[57.6%], +9[52.3%], | |
| | | | | | | | gain(15)(q11.2qter)[43.2%], del(16)(q21qter)[58.5%], | |
| | | | | | | | del(17)(p13.1pter)[53.0%], +18[43.5%], gain(22)(q11.21q12.3)[64.0%] | |
| chr9 | 500000 | 138000000 | 137500000 | pterqter | DUP | AML | del(3)(p11.2pter)[61.8%], +5[76.7%], | 0 |
| | | | | | | | del(5)(q11.2qter)[60.6%], −7[57.2%], +8[57.6%], +9[52.3%], | |
| | | | | | | | gain(15)(q11.2qter)[43.2%], del(16)(q21qter)[58.5%], | |
| | | | | | | | del(17)(p13.1pter)[53.0%], +18[43.5%], gain(22)(q11.21q12.3)[64.0%] | |
| chr15 | 23500000 | 101500000 | 7.80E+07 | q11.2qter | DUP | AML | del(3)(p11.2pter)[61.8%], +5[76.7%], | 0 |
| | | | | | | | del(5)(q11.2qter)[60.6%], −7[57.2%], +8[57.6%], +9[52.3%], | |
| | | | | | | | gain(15)(q11.2qter)[43.2%], del(16)(q21qter)[58.5%], | |
| | | | | | | | del(17)(p13.1pter)[53.0%], +18[43.5%], gain(22)(q11.21q12.3)[64.0%] | |
| chr13 | 67000000 | 113500000 | 46500000 | q21.32qter | DUP | AML | del(7)(q22.1qter)[60.0%], del(10)(q22.2q22.3)[58.6%], gain(13)(q21.32qter)[56.6%] | 0 |
| chr7 | 101500000 | 159000000 | 57500000 | q22.1qter | DEL | AML | del(7)(q22.1qter)[60.0%], del(10)(q22.2q22.3)[58.6%], gain(13)(q21.32qter)[56.6%] | 0 |
| chr10 | 75000000 | 79500000 | 4500000 | q22.2q22.3 | DEL | AML | del(7)(q22.1qter)[60.0%], del(10)(q22.2q22.3)[58.6%], gain(13)(q21.32qter)[56.6%] | 0 |
| chr13 | 60500000 | 62500000 | 2.00E+06 | q21.2q21.31 | DEL | AML | del(13)(q21.2q21.31)[96.6%] | 0 |
| chr11 | 117000000 | 135000000 | 1.80E+07 | q23.3qter | DUP | AML | gain(11)(q23.3qter)[15.0%] | inv(16)(q22.1p13.11)[36.5%] |
| chr1 | 3000000 | 248000000 | 2.45E+08 | p36.32qter | DUP | ALL | +1[44.6%], −2[21.4%], −3[21.6%], −4[22.0%], | 0 |
| | | | | | | | del(5)(q21.2qter)[20.7%], +6[42.6%], −7[21.7%], +8[42.3%], −9[20.8%], −12[21.3%], | |
| | | | | | | | del(13)(q12.11qter)[21.5%], | |
| | | | | | | | del(15)(q11.2qter)[20.7%], −16[19.7%], −17[19.2%], +18[42.1%], +19[47.4%], −20[19.1%], | |
| | | | | | | | gain(21)(q11.2qter)[43.2%], −X[2 | |
| chr12 | 500000 | 133000000 | 132500000 | pterqter | DEL | ALL | +1[44.6%], −2[21.4%], −3[21.6%], −4[22.0%], | 0 |
| | | | | | | | del(5)(q21.2qter)[20.7%], +6[42.6%], −7[21.7%], +8[42.3%], −9[20.8%], −12[21.3%], | |
| | | | | | | | del(13)(q12.11qter)[21.5%], | |
| | | | | | | | del(15)(q11.2qter)[20.7%], −16[19.7%], −17[19.2%], +18[42.1%], +19[47.4%], −20[19.1%], | |
| | | | | | | | gain(21)(q11.2qter)[43.2%], −X[2 | |
| chr13 | 20000000 | 113500000 | 93500000 | q12.11qter | DEL | ALL | +1[44.6%], −2[21.4%], −3[21.6%], −4[22.0%], | 0 |
| | | | | | | | del(5)(q21.2qter)[20.7%], +6[42.6%], −7[21.7%], +8[42.3%], −9[20.8%], −12[21.3%], | |
| | | | | | | | del(13)(q12.11qter)[21.5%], | |
| | | | | | | | del(15)(q11.2qter)[20.7%], −16[19.7%], −17[19.2%], +18[42.1%], +19[47.4%], −20[19.1%], | |
| | | | | | | | gain(21)(q11.2qter)[43.2%], −X[2 | |
| chr15 | 23500000 | 101500000 | 7.80E+07 | q11.2qter | DEL | ALL | +1[44.6%], −2[21.4%], −3[21.6%], −4[22.0%], | 0 |
| | | | | | | | del(5)(q21.2qter)[20.7%], +6[42.6%], −7[21.7%], +8[42.3%], −9[20.8%], −12[21.3%], | |
| | | | | | | | del(13)(q12.11qter)[21.5%], | |
| | | | | | | | del(15)(q11.2qter)[20.7%], −16[19.7%], −17[19.2%], +18[42.1%], +19[47.4%], −20[19.1%], | |
| | | | | | | | gain(21)(q11.2qter)[43.2%], −X[2 | |
| chr16 | 1500000 | 90000000 | 88500000 | pterqter | DEL | ALL | +1[44.6%], −2[21.4%], −3[21.6%], −4[22.0%], | 0 |
| | | | | | | | del(5)(q21.2qter)[20.7%], +6[42.6%], −7[21.7%], +8[42.3%], −9[20.8%], −12[21.3%], | |
| | | | | | | | del(13)(q12.11qter)[21.5%], | |
| | | | | | | | del(15)(q11.2qter)[20.7%], −16[19.7%], −17[19.2%], +18[42.1%], +19[47.4%], −20[19.1%], | |
| | | | | | | | gain(21)(q11.2qter)[43.2%], −X[2 | |
| chr17 | 500000 | 83000000 | 82500000 | pterqter | DEL | ALL | +1[44.6%], −2[21.4%], −3[21.6%], −4[22.0%], | 0 |
| | | | | | | | del(5)(q21.2qter)[20.7%], +6[42.6%], −7[21.7%], +8[42.3%], −9[20.8%], −12[21.3%], | |
| | | | | | | | del(13)(q12.11qter)[21.5%], | |
| | | | | | | | del(15)(q11.2qter)[20.7%], −16[19.7%], −17[19.2%], +18[42.1%], +19[47.4%], −20[19.1%], | |
| | | | | | | | gain(21)(q11.2qter)[43.2%], −X[2 | |
| chr18 | 500000 | 80000000 | 79500000 | pterqter | DUP | ALL | +1[44.6%], −2[21.4%], −3[21.6%], −4[22.0%], | 0 |
| | | | | | | | del(5)(q21.2qter)[20.7%], +6[42.6%], −7[21.7%], +8[42.3%], −9[20.8%], −12[21.3%], | |
| | | | | | | | del(13)(q12.11qter)[21.5%], | |
| | | | | | | | del(15)(q11.2qter)[20.7%], −16[19.7%], −17[19.2%], +18[42.1%], +19[47.4%], −20[19.1%], | |
| | | | | | | | gain(21)(q11.2qter)[43.2%], −X[2 | |
| chr19 | 1500000 | 58500000 | 5.70E+07 | pterqter | DUP | ALL | +1[44.6%], −2[21.4%], −3[21.6%], −4[22.0%], | 0 |
| | | | | | | | del(5)(q21.2qter)[20.7%], +6[42.6%], −7[21.7%], +8[42.3%], −9[20.8%], −12[21.3%], | |
| | | | | | | | del(13)(q12.11qter)[21.5%], | |
| | | | | | | | del(15)(q11.2qter)[20.7%], −16[19.7%], −17[19.2%], +18[42.1%], +19[47.4%], −20[19.1%], | |
| | | | | | | | gain(21)(q11.2qter)[43.2%], −X[2 | |
| chr2 | 500000 | 242000000 | 241500000 | pterqter | DEL | ALL | +1[44.6%], −2[21.4%], −3[21.6%], −4[22.0%], | 0 |
| | | | | | | | del(5)(q21.2qter)[20.7%], +6[42.6%], −7[21.7%], +8[42.3%], −9[20.8%], −12[21.3%], | |
| | | | | | | | del(13)(q12.11qter)[21.5%], | |
| | | | | | | | del(15)(q11.2qter)[20.7%], −16[19.7%], −17[19.2%], +18[42.1%], +19[47.4%], −20[19.1%], | |
| | | | | | | | gain(21)(q11.2qter)[43.2%], −X[2 | |
| chr20 | 500000 | 64000000 | 63500000 | pterqter | DEL | ALL | +1[44.6%], −2[21.4%], −3[21.6%], −4[22.0%], | 0 |
| | | | | | | | del(5)(q21.2qter)[20.7%], +6[42.6%], −7[21.7%], +8[42.3%], −9[20.8%], −12[21.3%], | |
| | | | | | | | del(13)(q12.11qter)[21.5%], | |
| | | | | | | | del(15)(q11.2qter)[20.7%], −16[19.7%], −17[19.2%], +18[42.1%], +19[47.4%], −20[19.1%], | |
| | | | | | | | gain(21)(q11.2qter)[43.2%], −X[2 | |
| chr21 | 14000000 | 46500000 | 32500000 | q11.2qter | DUP | ALL | +1[44.6%], −2[21.4%], −3[21.6%], −4[22.0%], | 0 |
| | | | | | | | del(5)(q21.2qter)[20.7%], +6[42.6%], −7[21.7%], +8[42.3%], −9[20.8%], −12[21.3%], | |
| | | | | | | | del(13)(q12.11qter)[21.5%], | |
| | | | | | | | del(15)(q11.2qter)[20.7%], −16[19.7%], −17[19.2%], +18[42.1%], +19[47.4%], −20[19.1%], | |
| | | | | | | | gain(21)(q11.2qter)[43.2%], −X[2 | |
| chr3 | 1000000 | 197500000 | 196500000 | pterqter | DEL | ALL | +1[44.6%], −2[21.4%], −3[21.6%], −4[22.0%], | 0 |
| | | | | | | | del(5)(q21.2qter)[20.7%], +6[42.6%], −7[21.7%], +8[42.3%], −9[20.8%], −12[21.3%], | |
| | | | | | | | del(13)(q12.11qter)[21.5%], | |
| | | | | | | | del(15)(q11.2qter)[20.7%], −16[19.7%], −17[19.2%], +18[42.1%], +19[47.4%], −20[19.1%], | |
| | | | | | | | gain(21)(q11.2qter)[43.2%], −X[2 | |
| chr4 | 500000 | 189500000 | 1.89E+08 | pterqter | DEL | ALL | +1[44.6%], −2[21.4%], −3[21.6%], −4[22.0%], | 0 |
| | | | | | | | del(5)(q21.2qter)[20.7%], +6[42.6%], −7[21.7%], +8[42.3%], −9[20.8%], −12[21.3%], | |
| | | | | | | | del(13)(q12.11qter)[21.5%], | |
| | | | | | | | del(15)(q11.2qter)[20.7%], −16[19.7%], −17[19.2%], +18[42.1%], +19[47.4%], −20[19.1%], | |
| | | | | | | | gain(21)(q11.2qter)[43.2%], −X[2 | |
| chr5 | 104000000 | 181000000 | 7.70E+07 | q21.2qter | DEL | ALL | +1[44.6%], −2[21.4%], −3[21.6%], −4[22.0%], | 0 |
| | | | | | | | del(5)(q21.2qter)[20.7%], +6[42.6%], −7[21.7%], +8[42.3%], −9[20.8%], −12[21.3%], | |
| | | | | | | | del(13)(q12.11qter)[21.5%], | |
| | | | | | | | del(15)(q11.2qter)[20.7%], −16[19.7%], −17[19.2%], +18[42.1%], +19[47.4%], −20[19.1%], | |
| | | | | | | | gain(21)(q11.2qter)[43.2%], −X[2 | |
| chr6 | 500000 | 170500000 | 1.70E+08 | pterqter | DUP | ALL | +1[44.6%], −2[21.4%], −3[21.6%], −4[22.0%], | 0 |
| | | | | | | | del(5)(q21.2qter)[20.7%], +6[42.6%], −7[21.7%], +8[42.3%], −9[20.8%], −12[21.3%], | |
| | | | | | | | del(13)(q12.11qter)[21.5%], | |
| | | | | | | | del(15)(q11.2qter)[20.7%], −16[19.7%], −17[19.2%], +18[42.1%], +19[47.4%], −20[19.1%], | |
| | | | | | | | gain(21)(q11.2qter)[43.2%], −X[2 | |
| chr7 | 500000 | 159000000 | 158500000 | pterqter | DEL | ALL | +1[44.6%], −2[21.4%], −3[21.6%], −4[22.0%], | 0 |
| | | | | | | | del(5)(q21.2qter)[20.7%], +6[42.6%], −7[21.7%], +8[42.3%], −9[20.8%], −12[21.3%], | |
| | | | | | | | del(13)(q12.11qter)[21.5%], | |
| | | | | | | | del(15)(q11.2qter)[20.7%], −16[19.7%], −17[19.2%], +18[42.1%], +19[47.4%], −20[19.1%], | |
| | | | | | | | gain(21)(q11.2qter)[43.2%], −X[2 | |
| chr8 | 500000 | 145000000 | 144500000 | pterqter | DUP | ALL | +1[44.6%], −2[21.4%], −3[21.6%], −4[22.0%], | 0 |
| | | | | | | | del(5)(q21.2qter)[20.7%], +6[42.6%], −7[21.7%], +8[42.3%], −9[20.8%], −12[21.3%], | |
| | | | | | | | del(13)(q12.11qter)[21.5%], | |
| | | | | | | | del(15)(q11.2qter)[20.7%], −16[19.7%], −17[19.2%], +18[42.1%], +19[47.4%], −20[19.1%], | |
| | | | | | | | gain(21)(q11.2qter)[43.2%], −X[2 | |
| chr9 | 500000 | 138000000 | 137500000 | pterqter | DEL | ALL | +1[44.6%], −2[21.4%], −3[21.6%], −4[22.0%], | 0 |
| | | | | | | | del(5)(q21.2qter)[20.7%], +6[42.6%], −7[21.7%], +8[42.3%], −9[20.8%], −12[21.3%], | |
| | | | | | | | del(13)(q12.11qter)[21.5%], | |
| | | | | | | | del(15)(q11.2qter)[20.7%], −16[19.7%], −17[19.2%], +18[42.1%], +19[47.4%], −20[19.1%], | |
| | | | | | | | gain(21)(q11.2qter)[43.2%], −X[2 | |
| chrX | 3000000 | 154000000 | 1.51E+08 | pterqter | DEL | ALL | +1[44.6%], −2[21.4%], −3[21.6%], −4[22.0%], | 0 |
| | | | | | | | del(5)(q21.2qter)[20.7%], +6[42.6%], −7[21.7%], +8[42.3%], −9[20.8%], −12[21.3%], | |
| | | | | | | | del(13)(q12.11qter)[21.5%], | |
| | | | | | | | del(15)(q11.2qter)[20.7%], −16[19.7%], −17[19.2%], +18[42.1%], +19[47.4%], −20[19.1%], | |
| | | | | | | | gain(21)(q11.2qter)[43.2%], −X[2 | |
| chr11 | 72500000 | 135000000 | 62500000 | q13.4qter | DUP | AML | del(2)(q36.3qter)[89.1%], gain(11)(q13.4qter)[90.2%] | 0 |
| chr2 | 227500000 | 242000000 | 14500000 | q36.3qter | DEL | AML | del(2)(q36.3qter)[89.1%], gain(11)(q13.4qter)[90.2%] | 0 |
| chr8 | 500000 | 39500000 | 3.90E+07 | pterp11.22 | DUP | MDS | gain(8)(p11.22pter)[160.7%], −8[82.0%], gain(8)(q12.3qter)[155.8%] | 0 |
| chr8 | 39500000 | 64000000 | 24500000 | p11.22q12.3 | DEL | MDS | gain(8)(p11.22pter)[160.7%], −8[82.0%], gain(8)(q12.3qter)[155.8%] | 0 |
| chr8 | 64000000 | 145000000 | 8.10E+07 | q12.3qter | DUP | MDS | gain(8)(p11.22pter)[160.7%], −8[82.0%], gain(8)(q12.3qter)[155.8%] | 0 |
| chr18 | 500000 | 14000000 | 13500000 | pterp11.21 | DEL | AML | gain(2)(q14.1qter)[23.6%], gain(7)(p12.3p14.1)[23.6%], −7[21.5%], | t(15; 17)(q24.1; q21.2)[3.3%], |
| | | | | | | | del(9)(p21.1pter)[17.4%], gain(9)(q22.33qter)[21.4%], del(10)(p11.21pter)[23.8%], | t(15; 17)(q24.1; q21.2)[4.1%] |
| | | | | | | | gain(10)(q11.21qter)[22.1%], del(13)(q12.11qter)[20.7%], del(16)(q23.1qter)[21.7%], | |
| | | | | | | | del(18)(p11.21pter)[15.2%], gain(19)(p12pter)[21.7%], gain(20)(q11.21qter)[35.8%] | |
| chr19 | 1500000 | 20000000 | 18500000 | pterp12 | DUP | AML | gain(2)(q14.1qter)[23.6%], gain(7)(p12.3p14.1)[23.6%], −7[21.5%], | t(15; 17)(q24.1; q21.2)[3.3%], |
| | | | | | | | del(9)(p21.1pter)[17.4%], gain(9)(q22.33qter)[21.4%], del(10)(p11.21pter)[23.8%], | t(15; 17)(q24.1; q21.2)[4.1%] |
| | | | | | | | gain(10)(q11.21qter)[22.1%], del(13)(q12.11qter)[20.7%], del(16)(q23.1qter)[21.7%], | |
| | | | | | | | del(18)(p11.21pter)[15.2%], gain(19)(p12pter)[21.7%], gain(20)(q11.21qter)[35.8%] | |
| chr20 | 31500000 | 64000000 | 32500000 | q11.21qter | DUP | AML | gain(2)(q14.1qter)[23.6%], gain(7)(p12.3p14.1)[23.6%], −7[21.5%], | t(15; 17)(q24.1; q21.2)[3.3%], |
| | | | | | | | del(9)(p21.1pter)[17.4%], gain(9)(q22.33qter)[21.4%], del(10)(p11.21pter)[23.8%], | t(15; 17)(q24.1; q21.2)[4.1%] |
| | | | | | | | gain(10)(q11.21qter)[22.1%], del(13)(q12.11qter)[20.7%], del(16)(q23.1qter)[21.7%], | |
| | | | | | | | del(18)(p11.21pter)[15.2%], gain(19)(p12pter)[21.7%], gain(20)(q11.21qter)[35.8%] | |
| chr7 | 38500000 | 48000000 | 9500000 | p14.1p12.3 | DUP | AML | gain(2)(q14.1qter)[23.6%], gain(7)(p12.3p14.1)[23.6%], −7[21.5%], | t(15; 17)(q24.1; q21.2)[3.3%], |
| | | | | | | | del(9)(p21.1pter)[17.4%], gain(9)(q22.33qter)[21.4%], del(10)(p11.21pter)[23.8%], | t(15; 17)(q24.1; q21.2)[4.1%] |
| | | | | | | | gain(10)(q11.21qter)[22.1%], del(13)(q12.11qter)[20.7%], del(16)(q23.1qter)[21.7%], | |
| | | | | | | | del(18)(p11.21pter)[15.2%], gain(19)(p12pter)[21.7%], gain(20)(q11.21qter)[35.8%] | |
| chr7 | 48000000 | 159000000 | 1.11E+08 | p12.3qter | DEL | AML | gain(2)(q14.1qter)[23.6%], gain(7)(p12.3p14.1)[23.6%], −7[21.5%], | t(15; 17)(q24.1; q21.2)[3.3%], |
| | | | | | | | del(9)(p21.1pter)[17.4%], gain(9)(q22.33qter)[21.4%], del(10)(p11.21pter)[23.8%], | t(15; 17)(q24.1; q21.2)[4.1%] |
| | | | | | | | gain(10)(q11.21qter)[22.1%], del(13)(q12.11qter)[20.7%], del(16)(q23.1qter)[21.7%], | |
| | | | | | | | del(18)(p11.21pter)[15.2%], gain(19)(p12pter)[21.7%], gain(20)(q11.21qter)[35.8%] | |
| chr13 | 20000000 | 113500000 | 93500000 | q12.11qter | DEL | AML | gain(2)(q14.1qter)[23.6%], gain(7)(p12.3p14.1)[23.6%], −7[21.5%], | t(15; 17)(q24.1; q21.2)[3.3%], |
| | | | | | | | del(9)(p21.1pter)[17.4%], gain(9)(q22.33qter)[21.4%], del(10)(p11.21pter)[23.8%], | t(15; 17)(q24.1; q21.2)[4.1%] |
| | | | | | | | gain(10)(q11.21qter)[22.1%], del(13)(q12.11qter)[20.7%], del(16)(q23.1qter)[21.7%], | |
| | | | | | | | del(18)(p11.21pter)[15.2%], gain(19)(p12pter)[21.7%], gain(20)(q11.21qter)[35.8%] | |
| chr16 | 74500000 | 90000000 | 15500000 | q23.1qter | DEL | AML | gain(2)(q14.1qter)[23.6%], gain(7)(p12.3p14.1)[23.6%], −7[21.5%], | t(15; 17)(q24.1; q21.2)[3.3%], |
| | | | | | | | del(9)(p21.1pter)[17.4%], gain(9)(q22.33qter)[21.4%], del(10)(p11.21pter)[23.8%], | t(15; 17)(q24.1; q21.2)[4.1%] |
| | | | | | | | gain(10)(q11.21qter)[22.1%], del(13)(q12.11qter)[20.7%], del(16)(q23.1qter)[21.7%], | |
| | | | | | | | del(18)(p11.21pter)[15.2%], gain(19)(p12pter)[21.7%], gain(20)(q11.21qter)[35.8%] | |
| chr9 | 500000 | 32500000 | 3.20E+07 | pterp21.1 | DEL | AML | gain(2)(q14.1qter)[23.6%], gain(7)(p12.3p14.1)[23.6%], −7[21.5%], | t(15; 17)(q24.1; q21.2)[3.3%], |
| | | | | | | | del(9)(p21.1pter)[17.4%], gain(9)(q22.33qter)[21.4%], del(10)(p11.21pter)[23.8%], | t(15; 17)(q24.1; q21.2)[4.1%1 |
| | | | | | | | gain(10)(q11.21qter)[22.1%], del(13)(q12.11qter)[20.7%], del(16)(q23.1qter)[21.7%], | |
| | | | | | | | del(18)(p11.21pter)[15.2%], gain(19)(p12pter)[21.7%], gain(20)(q11.21qter)[35.8%1 | |
| chr2 | 118000000 | 242000000 | 1.24E+08 | q14.1qter | DUP | AML | gain(2)(q14.1qter)[23.6%], gain(7)(p12.3p14.1)[23.6%], −7[21.5%], | t(15; 17)(q24.1; q21.2)[3.3%], |
| | | | | | | | del(9)(p21.1pter)[17.4%], gain(9)(q22.33qter)[21.4%], del(10)(p11.21pter)[23.8%], | t(15; 17)(q24.1; q21.2)[4.1%] |
| | | | | | | | gain(10)(q11.21qter)[22.1%], del(13)(q12.11qter)[20.7%], del(16)(q23.1qter)[21.7%], | |
| | | | | | | | del(18)(p11.21pter)[15.2%], gain(19)(p12pter)[21.7%], gain(20)(q11.21qter)[35.8%] | |
| chr9 | 99500000 | 138000000 | 38500000 | q22.33qter | DUP | AML | gain(2)(q14.1qter)[23.6%], gain(7)(p12.3p14.1)[23.6%], −7[21.5%], | t(15; 17)(q24.1; q21.2)[3.3%], |
| | | | | | | | del(9)(p21.1pter)[17.4%], gain(9)(q22.33qter)[21.4%], del(10)(p11.21pter)[23.8%], | t(15; 17)(q24.1; q21.2)[4.1%] |
| | | | | | | | gain(10)(q11.21qter)[22.1%], del(13)(q12.11qter)[20.7%], del(16)(q23.1qter)[21.7%], | |
| | | | | | | | del(18)(p11.21pter)[15.2%], gain(19)(p12pter)[21.7%], gain(20)(q11.21qter)[35.8%] | |
| chr10 | 500000 | 37000000 | 36500000 | pterp11.21 | DEL | AML | gain(2)(q14.1qter)[23.6%], gain(7)(p12.3p14.1)[23.6%], −7[21.5%], | t(15; 17)(q24.1; q21.2)[3.3%], |
| | | | | | | | del(9)(p21.1pter)[17.4%], gain(9)(q22.33qter)[21.4%], del(10)(p11.21pter)[23.8%], | t(15; 17)(q24.1; q21.2)[4.1%] |
| | | | | | | | gain(10)(q11.21qter)[22.1%], del(13)(q12.11qter)[20.7%], del(16)(q23.1qter)[21.7%], | |
| | | | | | | | del(18)(p11.21pter)[15.2%], gain(19)(p12pter)[21.7%], gain(20)(q11.21qter)[35.8%] | |
| chr10 | 43000000 | 133500000 | 90500000 | q11.21qter | DUP | AML | gain(2)(q14.1qter)[23.6%], gain(7)(p12.3p14.1)[23.6%], −7[21.5%], | t(15; 17)(q24.1; q21.2)[3.3%], |
| | | | | | | | del(9)(p21.1pter)[17.4%], gain(9)(q22.33qter)[21.4%], del(10)(p11.21pter)[23.8%], | t(15; 17)(q24.1; q21.2)[4.1%] |
| | | | | | | | gain(10)(q11.21qter)[22.1%], del(13)(q12.11qter)[20.7%], del(16)(q23.1qter)[21.7%], | |
| | | | | | | | del(18)(p11.21pter)[15.2%], gain(19)(p12pter)[21.7%], gain(20)(q11.21qter)[35.8%] | |
| chrY | 7000000 | 21000000 | 1.40E+07 | p11.2q11.223 | DEL | MDS | del(4)(q21.1q25)[53.3%], −Y[27.1%] | 0 |
| chr4 | 76000000 | 107000000 | 3.10E+07 | q21.1q25 | DEL | MDS | del(4)(q21.1q25)[53.3%], −Y[27.1%] | 0 |
| chrX | 3000000 | 9500000 | 6500000 | pterp22.31 | DUP | AML | del(5)(q31.2q31.2)[10.1%] | 0 |
| chr5 | 137577914 | 139513006 | 1935093 | q31.2q31.2 | DEL | AML | del(5)(q31.2q31.2)[10.1%] | 0 |
| chr19 | 2000000 | 58500000 | 56500000 | pterqter | DUP | AML | del(5)(q31.2q31.2)[10.1%] | 0 |
| chr5 | 1000000 | 30000000 | 2.90E+07 | pterp13.3 | DUP | AML | del(5)(q31.2q31.2)[10.1%] | 0 |
| chr7 | 92000000 | 159000000 | 6.70E+07 | q21.2qter | DEL | MDS | del(5)(q11.2qter)[63.2%], del(7)(q21.2qter)[63.2%], +8[43.1%1 | 0 |
| chr8 | 500000 | 145000000 | 144500000 | pterqter | DUP | MDS | del(5)(q11.2qter)[63.2%], del(7)(q21.2qter)[63.2%], +8[43.1%] | 0 |
| chr5 | 57500000 | 181000000 | 123500000 | q11.2qter | DEL | MDS | del(5)(q11.2qter)[63.2%], del(7)(q21.2qter)[63.2%], +8[43.1%] | 0 |
| chr21 | 41000000 | 46500000 | 5500000 | q22.2qter | DUP | AML | +4[7.7%], gain(21)(q22.2qter)[10.7%] | 0 |
| chr4 | 500000 | 189500000 | 1.89E+08 | pterqter | DUP | AML | +4[7.7%], gain(21)(q22.2qter)[10.7%] | 0 |
| chr13 | 31000000 | 104000000 | 7.30E+07 | q12.3q33.1 | DEL | ALL | +3[26.4%], del(7)(p11.2pter)[29.8%], +8[26.2%], del(13)(q12.3q33.1)[28.8%], | t(9; 22)(q34.12; q11.23)[8.1%], |
| | | | | | | | gain(14)(q11.2qter)[27.3%], +X[29.3%] | t(9; 22)(q34.12; q11.23)[10.3%] |
| chr8 | 500000 | 145000000 | 144500000 | pterqter | DUP | ALL | +3[26.4%], del(7)(p11.2pter)[29.8%], +8[26.2%], del(13)(q12.3q33.1)[28.8%], | t(9; 22)(q34.12; q11.23)[8.1%], |
| | | | | | | | gain(14)(q11.2qter)[27.3%], +X[29.3%] | t(9; 22)(q34.12; q11.23)[10.3%] |
| chrX | 3000000 | 154000000 | 1.51E+08 | pterqter | DUP | ALL | +3[26.4%], del(7)(p11.2pter)[29.8%], +8[26.2%], del(13)(q12.3q33.1)[28.8%], | t(9; 22)(q34.12; q11.23)[8.1%], |
| | | | | | | | gain(14)(q11.2qter)[27.3%], +X[29.3%] | t(9; 22)(q34.12; q11.23)[10.3%] |
| chr7 | 500000 | 54500000 | 5.40E+07 | pterp11.2 | DEL | ALL | +3[26.4%], del(7)(p11.2pter)[29.8%], +8[26.2%], del(13)(q12.3q33.1)[28.8%], | t(9; 22)(q34.12; q11.23)[8.1%], |
| | | | | | | | gain(14)(q11.2qter)[27.3%], +X[29.3%] | t(9; 22)(q34.12; q11.23)[10.3%] |
| chr14 | 20000000 | 105500000 | 85500000 | q11.2qter | DUP | ALL | +3[26.4%], del(7)(p11.2pter)[29.8%], +8[26.2%], del(13)(q12.3q33.1)[28.8%], | t(9; 22)(q34.12; q11.23)[8.1%], |
| | | | | | | | gain(14)(q11.2qter)[27.3%], +X[29.3%] | t(9; 22)(q34.12; q11.23)[10.3%] |
| chr3 | 1000000 | 197500000 | 196500000 | pterqter | DUP | ALL | +3[26.4%], del(7)(p11.2pter)[29.8%], +8[26.2%], del(13)(q12.3q33.1)[28.8%], | t(9; 22)(q34.12; q11.23)[8.1%], |
| | | | | | | | gain(14)(q11.2qter)[27.3%], +X[29.3%] | t(9; 22)(q34.12; q11.23)[10.3%] |
| chr3 | 66500000 | 82500000 | 1.60E+07 | p14.1p12.2 | DEL | AML | del(3)(p12.2p14.1)[76.6%], del(6)(p24.1pter)[75.1%], del(6)(q14.1q14.3)[77.6%], | 0 |
| | | | | | | | gain(8)(q12.1qter)[72.8%] | |
| chr6 | 75500000 | 84500000 | 9.00E+06 | q14.1q14.3 | DEL | AML | del(3)(p12.2p14.1)[76.6%], del(6)(p24.1pter)[75.1%], del(6)(q14.1q14.3)[77.6%], | 0 |
| | | | | | | | gain(8)(q12.1qter)[72.8%] | |
| chr6 | 70000000 | 115000000 | 4.50E+07 | q13q22.1 | DEL | AML | del(6)(p22.3pter)[13.1%], del(6)(q13q22.1)[12.2%] | t(15; 17)(q24.1; q21.2)[15.1%], |
| | | | | | | | | t(15; 17)(q24.1; q21.2)[19.5%] |
| chr6 | 500000 | 16000000 | 15500000 | pterp22.3 | DEL | AML | del(6)(p22.3pter)[13.1%], del(6)(q13q22.1)[12.2%] | t(15; 17)(q24.1; q21.2)[15.1%], |
| | | | | | | | | t(15; 17)(q24.1; q21.2)[19.5%] |
| chr9 | 20500000 | 33500000 | 1.30E+07 | p21.3p13.3 | DEL | AML | del(9)(p13.3p21.3)[11.6%] | 0 |
| chr18 | 500000 | 13000000 | 12500000 | pterp11.21 | DEL | MDS | −7[7.5%], del(18)(p11.21pter)[10.1%], del(18)(q21.2qter)[10.1%], +19[5.3%] | 0 |
| chr18 | 55000000 | 80000000 | 2.50E+07 | q21.2qter | DEL | MDS | −7[7.5%], del(18)(p11.21pter)[10.1%], del(18)(q21.2qter)[10.1%], +19[5.3%] | 0 |
| chr7 | 500000 | 159000000 | 158500000 | pterqter | DEL | MDS | −7[7.5%], del(18)(p11.21pter)[10.1%], del(18)(q21.2qter)[10.1%], +19[5.3%] | 0 |
| chr5 | 89000000 | 172000000 | 8.30E+07 | q14.3q35.1 | DEL | AML | del(5)(q14.3q35.1)[24.4%] | 0 |
| chrY | 7000000 | 21000000 | 1.40E+07 | p11.2q11.223 | DEL | MDS | −Y[21.4%] | 0 |
| chr9 | 68500000 | 105000000 | 36500000 | q21.11q31.1 | DEL | AML | del(9)(q21.11q31.1)[13.2%], −Y[15.4%] | t(8; 21)(q21.3; q22.12)[26.5%], |
| | | | | | | | | t(8; 21)(q21.3; q22.12)[27.9%] |
| chrY | 7000000 | 21000000 | 1.40E+07 | p11.2q11.223 | DEL | AML | del(9)(q21.11q31.1)[13.2%], −Y[15.4%1 | t(8; 21)(q21.3; q22.12)[26.5%], |
| | | | | | | | | t(8; 21)(q21.3; q22.12)[27.9%1 |
| chr4 | 134000000 | 139500000 | 5500000 | q28.3q31.1 | DEL | MDS | del(3)(q21.2q24)[83.4%], del(4)(q28.3q31.1)[65.6%], +8[82.7%] | 0 |
| chr3 | 119500000 | 197500000 | 7.80E+07 | q13.33qter | DUP | AML | gain(3)(q13.33qter)[8.1%], +8[8.4%] | 0 |
| chr10 | 500000 | 133500000 | 1.33E+08 | pterqter | DUP | AML | +8[27.6%], +10[29.3%] | t(15; 17)(q24.1; q21.2)[34.2%], |
| | | | | | | | | t(15; 17)(q24.1; q21.2)[28.8%] |
| chr8 | 500000 | 145000000 | 144500000 | pterqter | DUP | AML | +8[27.6%], +10[29.3%] | t(15; 17)(q24.1; q21.2)[34.2%], |
| | | | | | | | | t(15; 17)(q24.1; q21.2)[28.8%] |
| chr9 | 131000000 | 138000000 | 7.00E+06 | q34.12qter | DUP | ALL | gain(9)(q34.12qter)[58.2%], del(19)(p13.3pter)[53.8%], gain(22)(q11.21q11.23)[54.9%] | t(9; 22)(q34.12; q11.23)[32.6%], |
| | | | | | | | | t(9; 22)(q34.12; q11.23)[43.8%] |
| chr22 | 17500000 | 23500000 | 6.00E+06 | q11.21q11.23 | DUP | ALL | gain(9)(q34.12qter)[58.2%], del(19)(p13.3pter)[53.8%], gain(22)(q11.21q11.23)[54.9%] | t(9; 22)(q34.12; q11.23)[32.6%], |
| | | | | | | | | t(9; 22)(q34.12; q11.23)[43.8%] |
| chr11 | 23000000 | 42000000 | 1.90E+07 | p14.3p12 | DEL | AML | del(9)(q21.11q31.1)[87.3%], del(11)(p12p14.3)[87.6%] | t(15; 17)(q24.1; q21.2)[35.0%], |
| | | | | | | | | t(15; 17)(q24.1; q21.2)[32.5%] |
| chr13 | 47000000 | 53500000 | 6500000 | q14.2q14.3 | DEL | MDS | −7[83.9%], del(13)(q14.2q14.3)[85.3%] | 0 |
| chr12 | 10000000 | 15500000 | 5500000 | p13.2p12.3 | DEL | AML | del(5)(q21.1qter)[70.8%], −7[71.0%], del(12)(p12.3p13.2)[70.9%] | 0 |

TABLE S4
New SVs Identified by WGS

| Diagnosis | WGS.CNA.number | WGS.CNAs | WGS.Recurrent.SVs |
|---|---|---|---|
| AML | 0 | 0 | inv(16)(q22.1p13.11)[33.3%] |
| AML | 1 | +8[77.9%] | t(9; 11)(p21.3; q23.3)[34.5%], |
| | | | t(9; 11)(p21.3; q23.3)[26.2%] |
| AML | 1 | −X[88.4%] | t(10; 11)(p12.31; q23.3)[32.1%] |
| AML | 7 | +2[59.0%], +4[56.4%], +6[57.7%], +8[59.0%], | t(6; 11)(q27; q23.3)[37.9%], |
| | | gain(11)(q23.3qter)[71.0%], +19[146.2%], | t(6; 11)(q27; q23.3)[32.7%] |
| | | gain(21)(q11.2qter)[60.6%] | |
| AML | 1 | +8[84.9%] | t(10; 11)(p12.31; q23.3)[37.0%] |
| AML | 0 | 0 | t(11; 19)(q23.3; pter)[18.0%] |
| AML | 0 | 0 | t(7; 21)(p22.1; q22.12)[23.1%] |
| AML | 1 | +8[23.6%] | t(9; 11)(p21.3; q23.3)[19.7%], |
| | | | t(9; 11)(p21.3; q23.3)[20.8%] |
| AML | 1 | gain(21)(q11.2qter)[173.8%] | t(6; 11)(q27; q23.3)[28.8%] |
| AML | 0 | 0 | inv(16)(q22.1p13.11)[32.5%] |
| AML | 3 | −7[80.9%], +8[83.2%], | t(9; 11)(p21.3; q23.3)[19.3%], |
| | | del(12)(p12.2pter)[78.2%] | t(9; 11)(p21.3; q23.3)[18.4%] |
| AML | 1 | +8[74.5%] | t(9; 11)(p21.3; q23.3)[32.7%], |
| | | | t(9; 11)(p21.3; q23.3)[34.0%] |
| AML | 0 | 0 | t(11; 19)(q23.3; p13.11)[23.6%], |
| | | | t(11; 19)(q23.3; p13.11)[28.5%] |

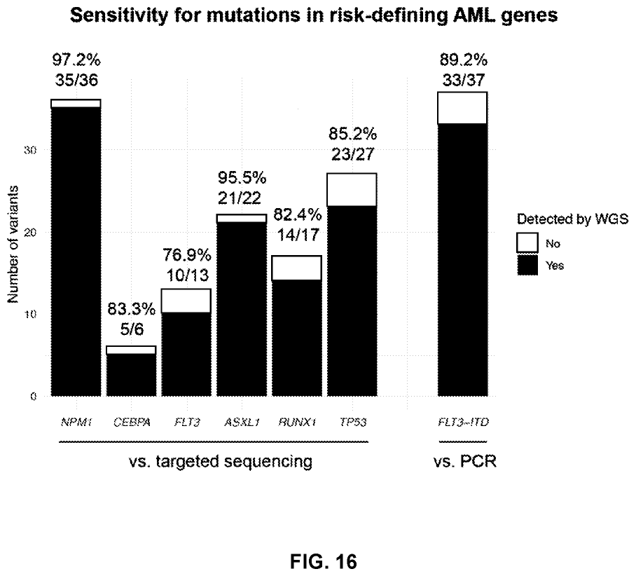

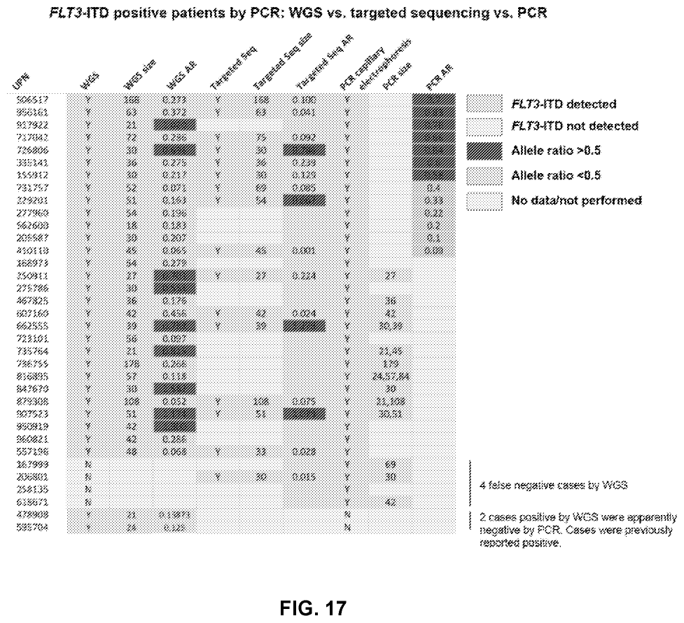

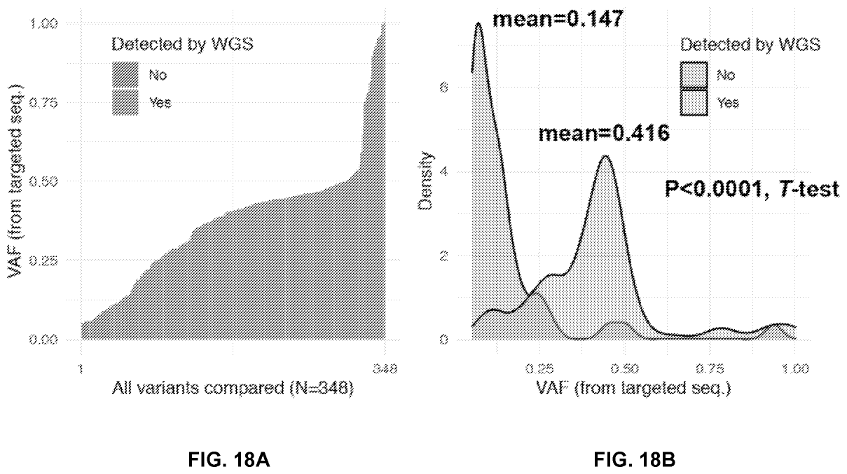

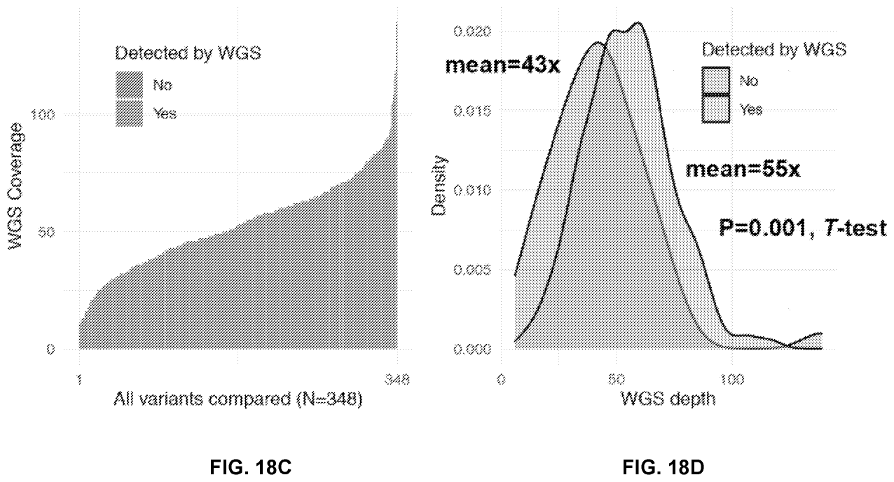

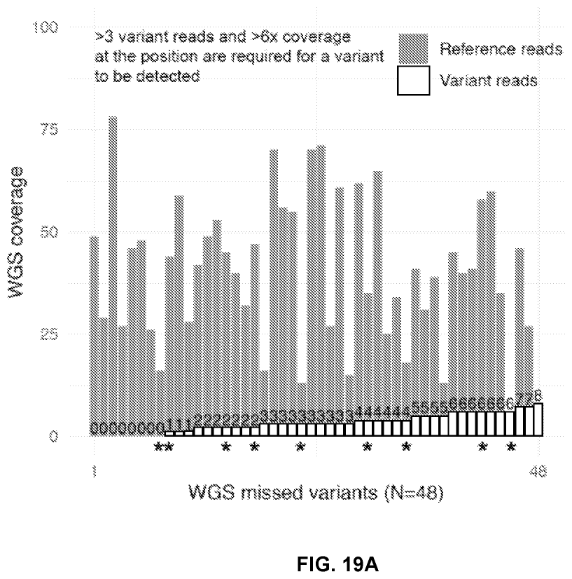

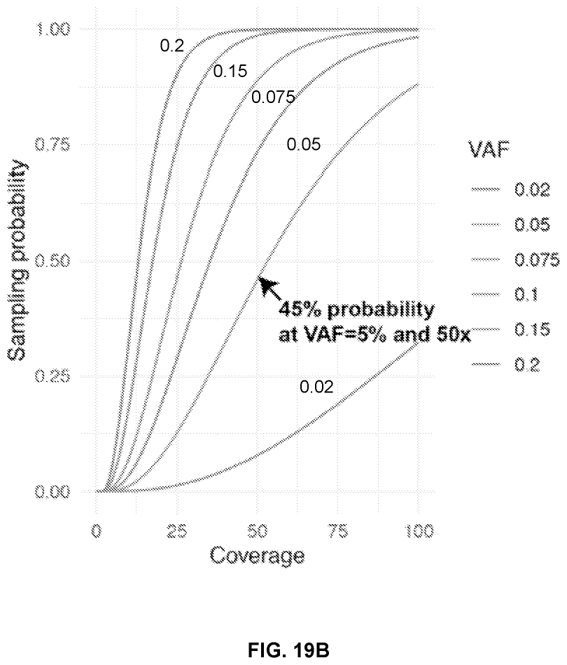

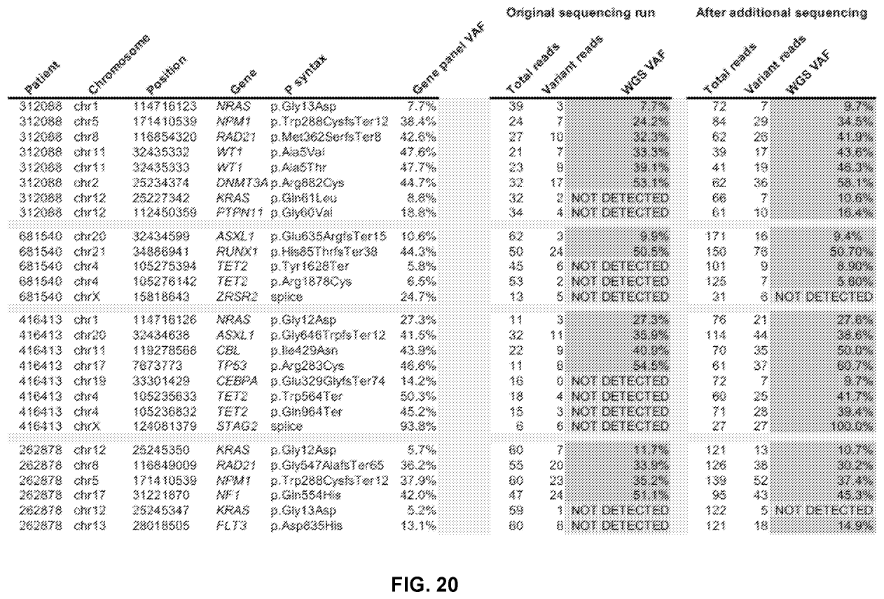

In a comparison of genetic mutations that were identified on whole genome sequencing with those that were identified on high-coverage (>500×) targeted clinical sequencing involving 102 patients, we found sensitivities of 84.6% for single-nucleotide variants and 91.5% for insertion-deletion (indel) mutations, along with a positive predictive value of more than 99% for variants with a minimum variant allele fraction of 5% (FIG. 6A). Similar performance was observed when considering only mutations in genes necessary for risk stratification in patients with AML, including a combined sensitivity of 87.5% for single-nucleotide variants and indels in ASXL1, CEBPA, FLT3, NPM1, RUNX1, and TP53 (FIGS. 16 and 17). False negatives occurred either because the variants were in subclones or were at low coverage positions on whole-genome sequencing (FIGS. 18A, 18B, 18C, 18D, 19A, and 19B); such variants were more readily detected with higher coverage sequencing (FIG. 20).

Clinical Feasibility and Diagnostic Yield

We evaluated the feasibility of using whole-genome sequencing for routine clinical testing by prospectively sequencing samples obtained from 117 consecutive patients. For this cohort, whole-genome sequencing was performed in weekly batches with a median batch size of 4 (range, 1 to 11) with the use of bone marrow aspirate samples submitted for karyotyping and FISH studies. The median total processing time was 5.1 days, which included 2 days for library preparation, 2 days for sequencing, and less than 1 day for analysis (FIG. 7A). The shortest times were about 3 days (approximately 78 hours), when clinical laboratory staffing allowed samples to be sequenced in dedicated sequencing runs immediately after library generation. Sequencing was successful in all the samples, and only 5 samples (4.3%) had less than 25× genome coverage in a single assay run. Seven samples required manual review of the automated copy-number alteration calls, with the remaining 110 samples (94.0%) needing no additional interventions to finalize the sequencing report.

This set of consecutive patients was also evaluated to estimate the diagnostic yield from whole-genome sequencing as compared with testing with cytogenetic analysis and targeted sequencing. This analysis was performed separately in samples obtained from patients with AML and in those obtained from patients with MDS. In the AML samples, the comparisons included clinical results from a standard FISH panel along with cytogenetic analysis and targeted sequencing to provide a realistic estimate of the expected yield of whole-genome sequencing. In this prospective cohort, results from conventional cytogenetic analysis and FISH assays in the 68 patients with AML resulted in the diagnosis of acute promyelocytic leukemia with the fusion gene PML-RARA in 5 patients and in the assignment of 27 patients to the adverse-risk group, 10 to the intermediate-risk group, and 19 to the favorable-risk group on the basis of established guidelines; 7 patients had unsuccessful or inconclusive results on cytogenetic analysis and could not be assigned to a risk group. Four patients were assigned to risk groups solely on the basis of positive FISH results for either PML-RARA (1 patient) or del(5q) (3 patients) (FIG. 7B).

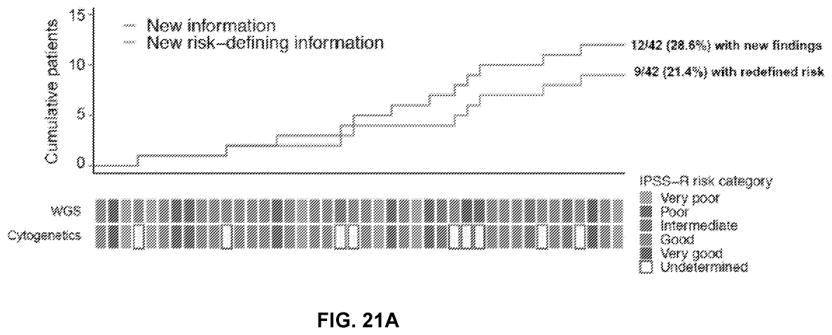

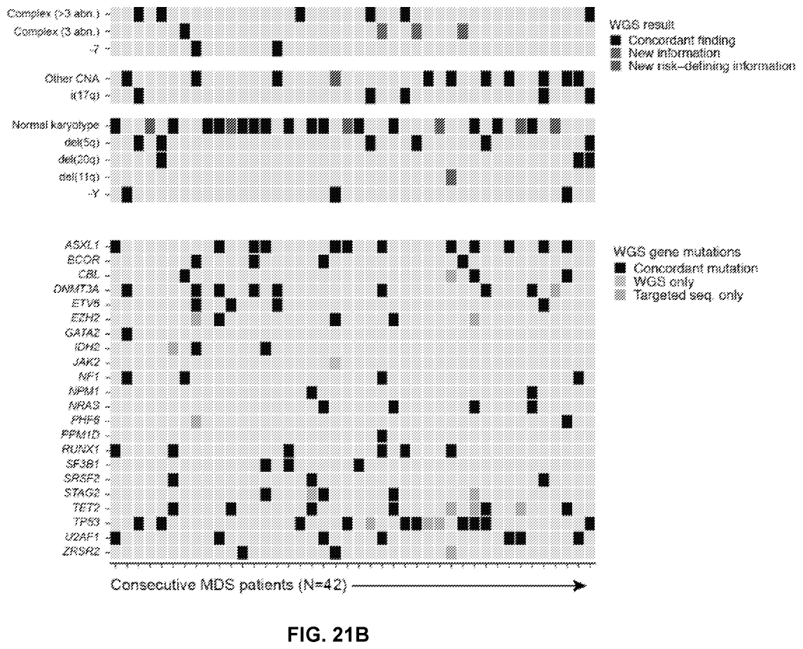

Whole-genome sequencing that was performed in the same cohort identified new abnormalities in 17 of 68 patients (25%). These abnormalities included cryptic or complex chromosomal rearrangements in 5 patients, new copy-number alterations that resulted in a complex karyotype in 4 patients, and identification of either a normal karyotype (in 4 patients) or 1 or 2 cytogenetic abnormalities in patients with inconclusive or unsuccessful results by cytogenetic analysis (in 4 patients). Using data only from whole-genome sequencing and a PCR assay for FLT3-ITD, we reclassified 10 of 68 patients without acute promyelocytic leukemia (15%) to a risk group that differed from the one that was based on conventional testing (FIG. 21A). A similar yield was observed for the 42 prospective patients with MDS, of whom 12 (29%) had inconclusive results on cytogenetic analysis or new findings on whole-genome sequencing, and 9 (21%) were assigned to a new IPSS-R risk category, which brings the combined number of patients with a reclassified risk-group assignment to 19 of all 117 patients (16.2%) who were included in this prospective cohort.

Predictive Value Using Existing Genetic-Risk Categories

We next asked whether whole-genome sequencing could be used in place of cytogenetic analysis to predict clinical outcomes using existing genetic risk groups. To avoid the confounding effect of hematopoietic stem-cell transplantation on outcome, we focused our analysis on 71 patients with AML who did not undergo this procedure, including 41 prospective and 30 retrospective patients; 58 patients (82%) received intensive induction chemotherapy, whereas the remaining 13 were treated with hypomethylating agents. These patients were assigned to a genetic risk group on the basis of whole-genome sequencing alone or conventional testing (the combined results of cytogenetic analysis, clinical FISH results, and targeted sequencing). The FLT3-ITD mutational status based on a PCR assay was used in the two classifications.

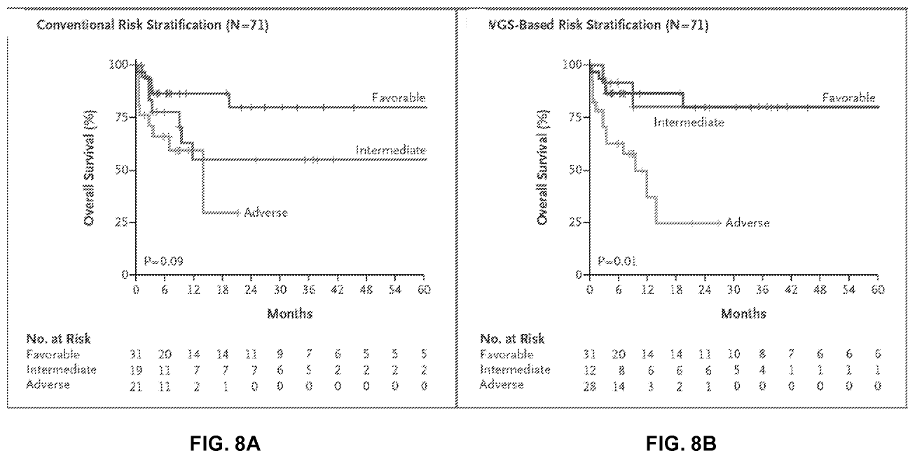

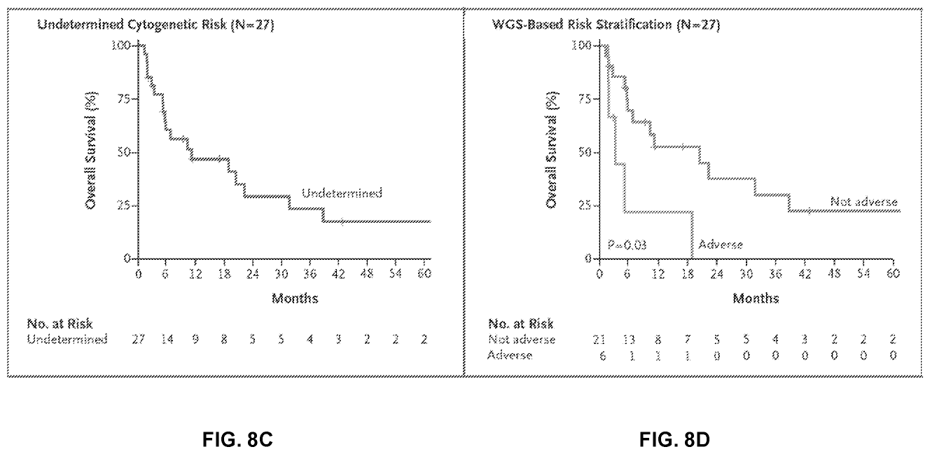

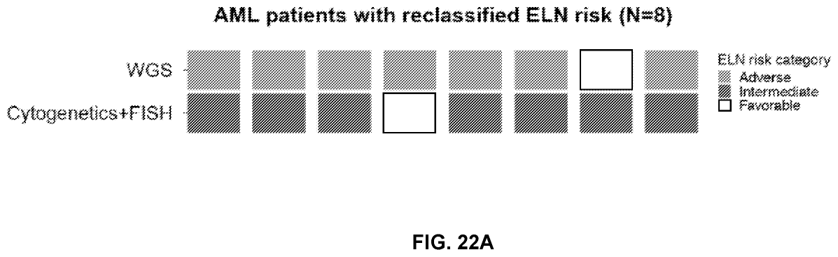

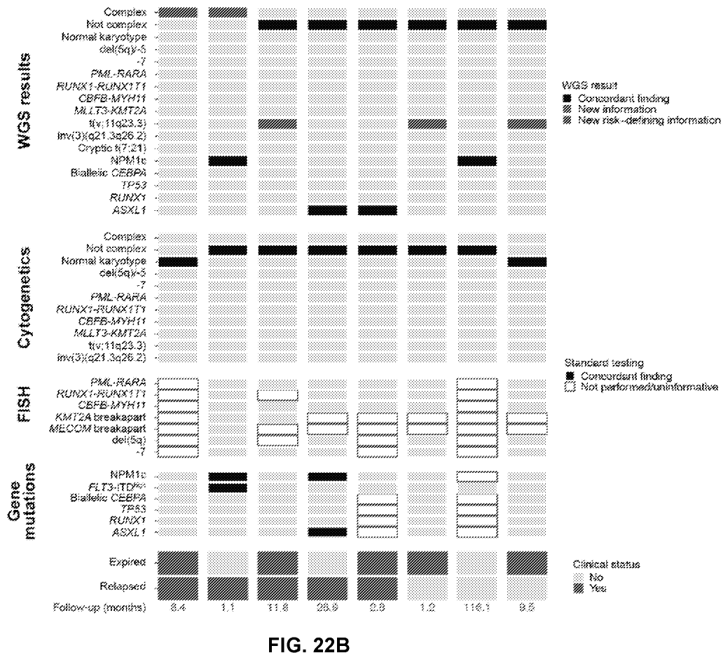

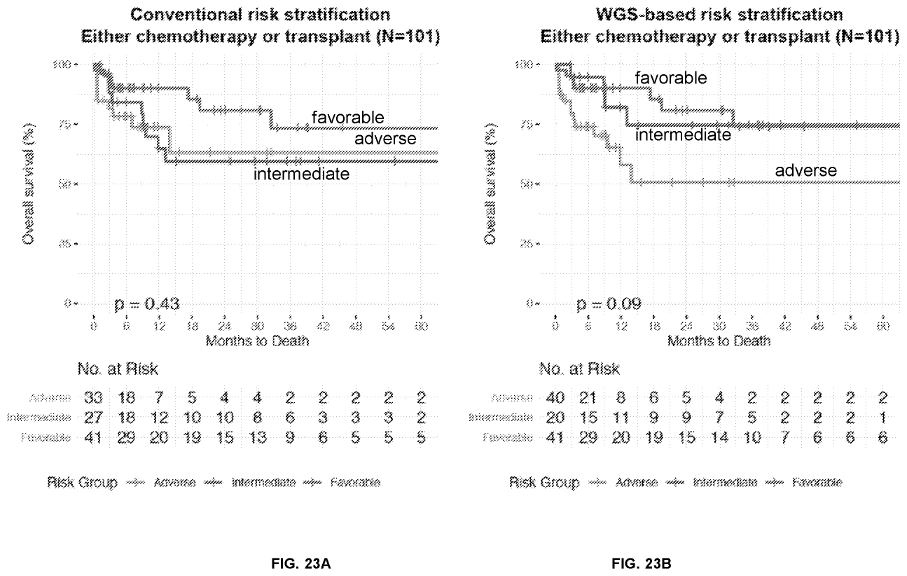

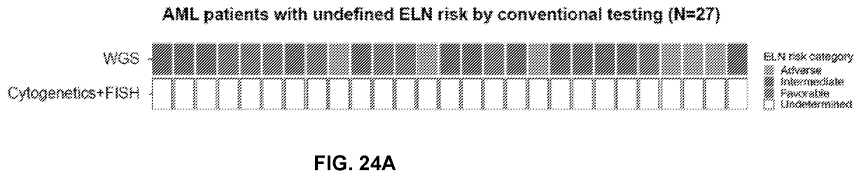

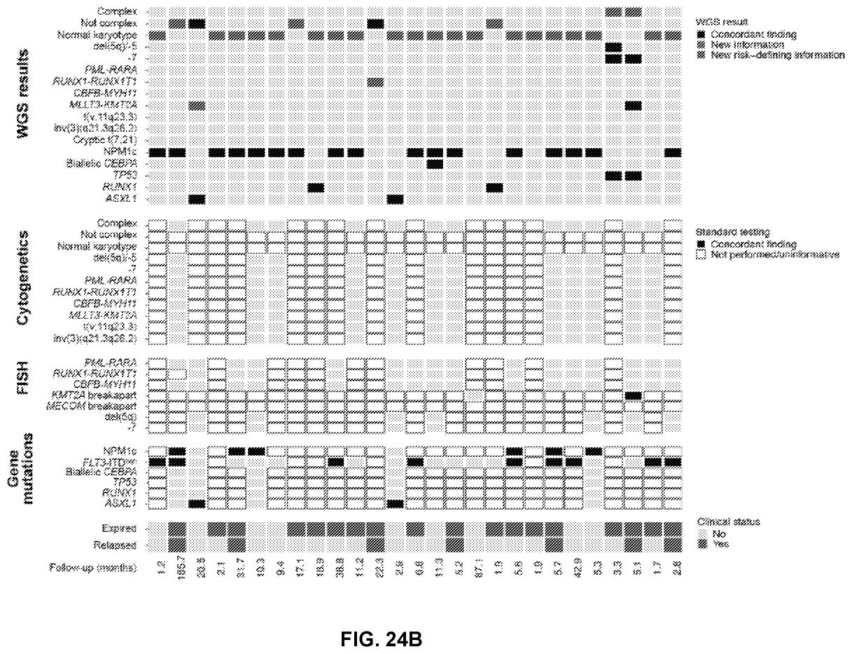

Assignments that were based on conventional testing were in agreement with the results on whole-genome sequencing for 63 of 71 patients (89%); 8 patients were reassigned to a different risk category, including 5 who had new adverse-risk findings that were identified by whole-genome sequencing (FIG. 22A). Risk groups that were defined according to the two methods had the expected associations with overall survival (adjusted P=0.09 by log-rank test in groups identified by conventional testing; adjusted P=0.01 by log-rank test in groups identified by whole-genome sequencing) (FIGS. 8A and 8B). Whole-genome sequencing provided slightly better identification of patients with adverse risk and poor outcomes than conventional testing, with a hazard ratio for death of 0.32 (95% confidence interval [CI], 0.11 to 0.92) on age-adjusted Cox regression analysis, as compared with a hazard ratio of 0.66 (95% CI, 0.17 to 1.05) by conventional risk-group analysis. Similar results were observed in a larger cohort of 101 patients who were treated with either consolidation chemotherapy or stem-cell transplantation (FIGS. 23A and 24B).

We reasoned that whole-genome sequencing could have the greatest benefit for patients for whom cytogenetic results are unavailable at diagnosis, which occurs in up to 20% of patients with AML. Thus, we used whole-genome sequencing to evaluate 27 patients with AML who were not treated with stem cell transplantation (of whom 22 received standard induction chemotherapy), who could not be assigned to a risk group at the time of diagnosis because of unsuccessful cytogenetic analysis (in 6 patients), inconclusive results (in 13), or unknown results (in 8), and who had no reports of risk-defining events by FISH. The mean age at diagnosis in this cohort was similar to that of patients with defined cytogenetic risk (60.8 years and 54.7 years, respectively), and the median overall survival was 11.2 months (95% CI, 5.6 to 38.8) (FIG. 8C). Whole-genome sequencing analysis identified risk-defining chromosomal abnormalities in 4 patients, including KMT2A and RUNX1-RUNXT1 rearrangements in 1 patient each or a complex karyotype in 2 patients; the remaining 23 patients had either a normal karyotype or one or two abnormalities and were assigned to a risk category on the basis of mutations identified by whole-genome sequencing (FIG. 24).

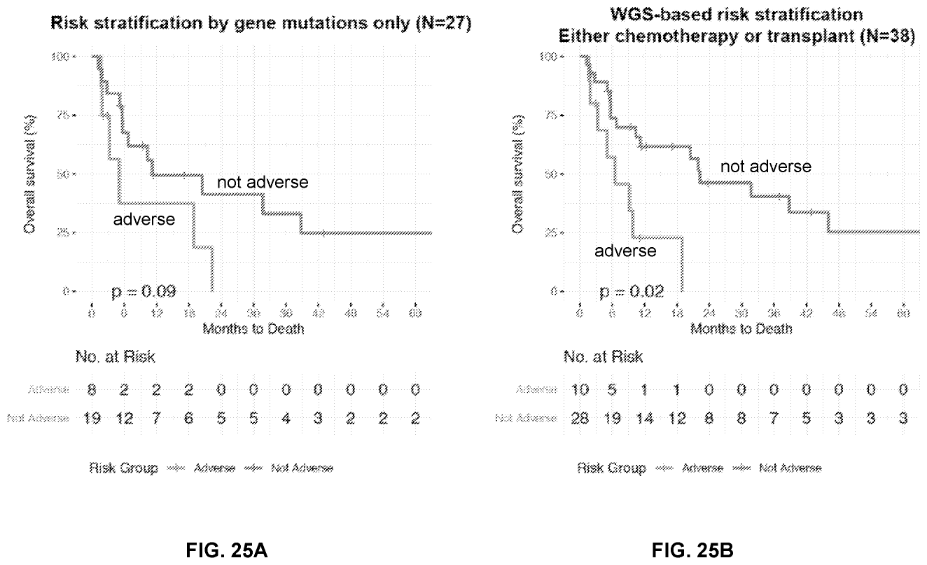

Survival analysis of these patients showed that risk predictions that were based on whole-genome sequencing also correlated with outcomes, with significantly longer overall survival in 21 patients with intermediate or favorable risk (median survival, 20.5 months; 95% CI, 5.6 to 38.8) than in 6 patients with adverse risk (median survival, 3.3 months; 95% CI, 1.7 to 18.9; adjusted P=0.03 by log-rank test) (FIG. 8D); hazard ratio of 0.29 (95% CI, 0.09 to 0.94) by age-adjusted Cox regression analysis. This survival difference was superior to that resulting from the assignment of patients to risk groups on the basis of gene mutations alone (FIG. 25A) and was maintained when 11 additional patients with inconclusive results on cytogenetic analysis who underwent allogeneic stem-cell transplantation were included in this cohort (total of 38 patients) (FIG. 25B)

The above non-limiting example is provided to further illustrate the present disclosure. It should be appreciated by those of skill in the art that the techniques disclosed in the examples represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

What is claimed is:

1. A computer-implemented method for the identification of clinically relevant structural variants in a subject with AML or MDS from whole genome sequencing data, the method comprising:
a. providing a whole-genome sequencing dataset, the whole-genome sequencing dataset comprising a plurality of alignments of tumor DNA sequence fragments to a reference human genome to a computing device;
b. performing, using the computing device, a structural variant analysis on the whole-genome sequencing dataset, the structural variant analysis including copy-number alteration (CNA) identification, structural variant (SV) identification, and gene-level variant identification to identify clinically relevant structural variants indicative of AML or MDS within the whole-genome sequencing dataset; and
c. producing, using the computing device, a report comprising the clinically relevant CNAs, SVs, and gene-level variants identified by the structural variant analysis.

2. The method of claim 1, wherein copy-number alteration (CNA) identification further comprises:
a. transforming, using the computing device, the alignments of the whole-genome sequencing dataset into a plurality of read counts over 500,000 bp nonoverlapping windows across the genome;
b. transforming, using the computing device, the plurality of read counts into a plurality of CNAs; and
c. filtering, using the computing device, plurality of CNAs to retain only CNAs greater than 5 Mbp,

3. The method of claim 1, wherein SV identification further comprises:
a. transforming, using the computing device, the alignments of the whole-genome sequencing dataset into a plurality of SV calls;
b. filtering, using the computing device, the plurality of SVs to retain only SV calls greater than 100 kbp in length; and
c. filtering, using the computing device, the SV calls greater than 100 kbp in length to identify translocations, deletions, duplications, and inversions that overlap a predefined list of recurrent and/or risk-defining SVs associated with AML or MDS.

4. The method of claim 1, wherein gene-level variant identification further comprises identifying, using the computing device, the alignments of the whole-genome sequencing dataset within about 85 kbp targeting 40 predetermined genes and gene hotspots that are recurrently mutated in AML or MDS.

5. The method of claim 1, wherein the clinically relevant CNAs, SVs, and gene-level variants identified by the structural variant analysis are indicative of a clinical outcome of the subject.

6. The method of claim 1, wherein providing the whole-genome sequencing dataset whole genome sequencing data further comprising performing whole-genome sequencing on a biological sample comprising tumor DNA from the subject with about 60× genome coverage.
